(19) United States
(12) Patent Application Publication
(10) Pub. No.: US 2024/0150388 A1
(43) Pub. Date: May 9, 2024

(54) HETEROBIFUNCTIONAL INHIBITORS OF E-SELECTIN AND GALECTIN-3

(71) Applicant: GLYCOMIMETICS, INC., Rockville, MD (US)

(72) Inventors: John L. MAGNANI, Gaithersburg, MD (US); John M. PETERSON, Slate Hill, NY (US); Arun K. SARKAR, North Potomac, MD (US); Yusufbhai U. VOHRA, Germantown, MD (US); Indranath GHOSH, Olney, MD (US); Jason NOGUEIRA, Germantown, MD (US)

(73) Assignee: GLYCOMIMETICS, INC., Rockville, MD (US)

(21) Appl. No.: 18/503,499

(22) Filed: Nov. 7, 2023

Related U.S. Application Data
(63) Continuation of application No. 17/418,579, filed on Jun. 25, 2021, now Pat. No. 11,845,771.
(60) Provisional application No. 62/785,565, filed on Dec. 27, 2018.

(51) Int. Cl. C07H 19/056

(52) U.S. Cl. CPC C07H 19/056

(57) ABSTRACT

Compounds, compositions, and methods for treatment and/or prevention of at least one disease, disorder, and/or condition by inhibiting binding of an E-selectin, galectin-3, or E-selectin and galectin-3 to ligands are disclosed. For example, heterobifunctional inhibitors of E-selectin and galectin-3 are described and pharmaceutical compositions comprising at least one such agent is described.

This application claims the benefit under 35 U.S.C. § 119(e) to U.S. Provisional Application No. 62/785,565 filed Dec. 27, 2018, which application is incorporated by reference herein in its entirety.

Compounds, compositions, and methods for treating and/or preventing at least one disease, disorder, and/or condition associated with E-selectin, galectin-3, or E-selectin and galectin-3 activity including, for example, inflammatory diseases, fibrosis, and cancers, are disclosed herein.

When a tissue is infected or damaged, the inflammatory process directs leukocytes and other immune system components to the site of infection or injury. Within this process, leukocytes play an important role in the engulfment and digestion of microorganisms. The recruitment of leukocytes to infected or damaged tissue is critical for mounting an effective immune defense.

Selectins are a group of structurally similar cell surface receptors important for mediating leukocyte binding to endothelial cells. These proteins are type 1 membrane proteins and are composed of an amino terminal lectin domain, an epidermal growth factor (EGF)-like domain, a variable number of complement receptor related repeats, a hydrophobic domain spanning region and a cytoplasmic domain. The binding interactions appear to be mediated by contact of the lectin domain of the selectins and various carbohydrate ligands.

There are three known selectins: E-selectin, P-selectin, and L-selectin. E-selectin is found on the surface of activated endothelial cells, which line the interior wall of capillaries. E-selectin binds to the carbohydrate sialyl-Lewis^{x} (sLe^{x}), which is presented as a glycoprotein or glycolipid on the surface of certain leukocytes (monocytes and neutrophils) and helps these cells adhere to capillary walls in areas where surrounding tissue is infected or damaged; and E-selectin also binds to sialyl-Lewis^{a} (sLe^{a}), which is expressed on many tumor cells. P-selectin is expressed on inflamed endothelium and platelets, and also recognizes sLe^{x} and sLe^{a}, but also contains a second site that interacts with sulfated tyrosine. The expression of E-selectin and P-selectin is generally increased when the tissue adjacent to a capillary is infected or damaged. L-selectin is expressed on leukocytes. Selectin-mediated intercellular adhesion is an example of a selectin-mediated function.

Although selectin-mediated cell adhesion is required for fighting infection and destroying foreign material, there are situations in which such cell adhesion is undesirable or excessive, resulting in tissue damage instead of repair. For example, many pathologies (such as autoimmune and inflammatory diseases, shock and reperfusion injuries) involve abnormal adhesion of white blood cells. Such abnormal cell adhesion may also play a role in transplant and graft rejection. In addition, some circulating cancer cells appear to take advantage of the inflammatory mechanism to bind to activated endothelium and metastasize. In such circumstances, modulation of selectin-mediated intercellular adhesion may be desirable

Modulators of selectin-mediated function include the PSGL-1 protein (and smaller peptide fragments), fucoidan, glycyrrhizin (and derivatives), sulfated lactose derivatives, heparin and heparin fragments, sulfated hyaluronic acid, chondroitin sulfate, sulfated dextran, sulfatides, and particular glycomimetic compounds (see, e.g., U.S. RE44,778). To date, all but the glycomimetics have shown to be unsuitable for drug development due to insufficient activity, toxicity, lack of specificity, poor ADME characteristics, and/or availability of material.

Galectins are proteins with a characteristic carbohydrate recognition domain (CRD) (Barondes, S. I., Cooper, D N. W., Gitt, M. A., and Leffler, H. (1994). Galectins. Structure and function of a large family of animal lectins. J. Biol. Chem. 269:20807-20810; Leffler, H., Carlsson, S., Hedlund, M., Qian, Y. and Poirier, F (2004) Introduction to galectins. Glycoconj. J. 19: 433-440). Galectin subunits can contain either one or two CRDs within a single peptide chain. The mono-CRDs galectins can occur as monomers or dimers in vertebrates. Galectin-3 is a monomer in solution but may aggregate and become multimeric upon encounter with ligands. Galectins are synthesized as cytosolic proteins. Evidence suggests roles for galectins in inflammation, fibrosis, cancer, and other disorders (see, e.g., U.S. Pat. No. 7,638,623).

A pro-inflammatory role of galectin-3 is indicated by its induction in cells at inflammatory sites, effects on immune cells, and decrease of the inflammatory response shown in animal models. Inflammation is a protective response of the body to invading organisms and tissue injury. However, if unbalanced, frequently it is also destructive and occurs as part of the pathology in many diseases. Because of this, there is great medical interest in pharmacological modulation of galectin-3 mediated inflammation.

Immunohistochemical studies show changed expression of certain galectins in cancer. Direct evidence for a role of galectin-3 in cancer comes from mouse models. In paired tumor cell lines (with decreased or increased expression of galectin-3), the induction of galectin-3 gives more tumors and metastasis and suppression of galectin-3 gives less tumors and metastasis. Galectin-3 has been proposed to enhance tumor growth by being anti-apoptotic, promote angiogenesis, or to promote metastasis by affecting cell adhesion.

Both natural and synthetic modulators of galectin-3 have been identified. However, natural compounds that have been identified as galectin-3 ligands are not suitable for use as active components in pharmaceutical compositions, because they have been reported to have low activity and specificity for galectins and galectin-3. As natural products they are difficult to produce as well-characterized drugs and are susceptible to acidic hydrolysis in the stomach and to enzymatic degradation. In addition, previously identified natural galectin-3 modulators are large and hydrophilic in nature, and are not readily absorbed from the gastrointestinal tract following oral administration.

Accordingly, there is a need in the art for inhibitors of E-selectin, galectin-3, and/or both E-selectin and galectin-3 mediated function. The present disclosure may fulfill one or more of these needs and/or may provide other advantages.

Compounds, compositions, and methods for treating and/or preventing (i.e., reducing the likelihood of occurrence or reoccurrence) at least one disease, disorder, and/or condition in which inhibiting binding of E-selectin, galectin-3, or both E-selectin and galectin-3 to one or more ligands may play a role are disclosed. Compounds disclosed herein are heterobifunctional, wherein an E-selectin inhibitor is linked to a galectin-3 inhibitor.

Disclosed is at least one compound chosen from compounds of Formula (I):

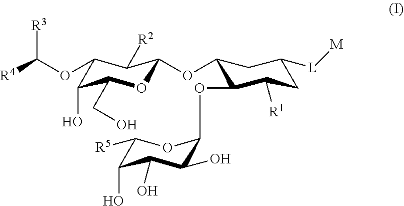

prodrugs of compounds of Formula (I), and pharmaceutically acceptable salts of any of the foregoing, wherein R¹, R², R³, R⁴, R⁵, L, and M are defined herein.

As used herein, “compound of Formula (I)” includes compounds of Formula (I), pharmaceutically acceptable salts of compounds of Formula (I), prodrugs of compounds of Formula (I), and pharmaceutically acceptable salts of prodrugs of compounds of Formula (I).

In some embodiments, compositions comprising at least one compound of Formula (I) and optionally at least one additional pharmaceutically acceptable ingredient are presented. In some embodiments, the compositions are pharmaceutical compositions.

In some embodiments, a method for treatment and/or prevention of at least one disease, disorder, and/or condition where inhibition of E-selectin, galectin-3, or both E-selectin and galectin-3 mediated functions is useful is disclosed, the method comprising administering to a subject in need thereof an effective amount of at least one compound of Formula (I) and/or a pharmaceutical composition comprising at least one compound of Formula (I).

In the following description, certain specific details are set forth in order to provide a thorough understanding of various embodiments. However, one skilled in the art will understand that the disclosed embodiments may be practiced without these details. In other instances, well-known structures have not been shown or described in detail to avoid unnecessarily obscuring descriptions of the embodiments. These and other embodiments will become apparent upon reference to the following detailed description and attached drawings.

### BRIEF DESCRIPTION OF THE DRAWINGS

FIG. 1 is a diagram illustrating the synthesis of building block 14.

FIG. 2 is a diagram illustrating the synthesis of building block 37.

FIG. 3 is a diagram illustrating the synthesis of building block 44.

FIG. 4 is a diagram illustrating the synthesis of building block 49.

FIG. 5 is a diagram illustrating the synthesis of compound 51.

FIG. 6 is a diagram illustrating the synthesis of compound 87.

FIG. 7 is a diagram illustrating the synthesis of compound 83.

FIG. 8 is a diagram illustrating the synthesis of compound 86.

Disclosed herein are heterobifunctional inhibitors of E-selectin and galectin-3, pharmaceutical compositions comprising the same, and methods for inhibiting E-selectin and/or galectin-3 mediated functions using the same. The compounds and compositions of the present disclosure may be useful for treating and/or preventing at least one disease, disorder, and/or condition that is treatable by inhibiting binding of E-selectin, galectin-3, or both E-selectin and galectin-3 to one or more ligands.

In some embodiments, disclosed is at least one compound chosen from compound of Formula (I):

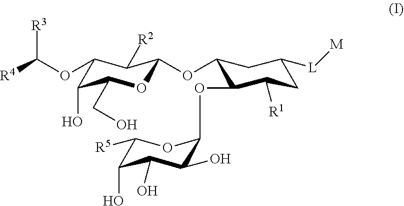

prodrugs of compounds of Formula (I), and pharmaceutically acceptable salts of any of the foregoing,
wherein
R¹ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl,

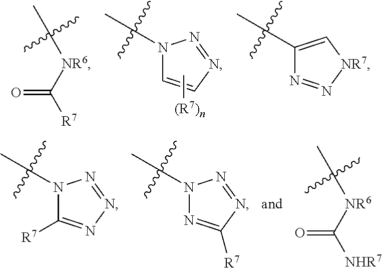

groups, wherein n is chosen from integers ranging from 0 to 2, R⁶ is chosen from H, C₁₋₈ alkyl, C₂₋₈ alkenyl, C₂₋₈ alkynyl, C₄₋₁₆ cycloalkylalkyl, and —C(═O)R⁷ groups, and each R⁷ is independently chosen from H, C₁₋₈ alkyl, C₂₋₈ alkenyl, C₂₋₈ alkynyl, C₄₋₁₆ cycloalkylalkyl, C₆₋₁₈ aryl, and C₁₋₁₃ heteroaryl groups;
R² is chosen from —OH, —OY¹, halo, —NH₂, —NY¹Y², —OC(═O)Y¹, —NHC(═O)Y¹, and —NHC(═O)NHY¹ groups, wherein Y¹ and Y², which may be the same or different, are independently chosen from C₁₋₈ alkyl, C₂₋₈ alkenyl, C₂₋₈ alkynyl, C₄₋₁₆ cycloalkylalkyl, C₂₋₁₂ heterocyclyl, C₆₋₁₈ aryl, and C₁₋₁₃ heteroaryl groups, or Y¹ and Y² join together along with the nitrogen atom to which they are attached to form a ring;
R³ is chosen from —CN, —CH₂CN, and —C(═O)Y³ groups, wherein Y³ is chosen from C₁₋₈ alkyl, C₂₋₈ alkenyl, C₂₋₈ alkynyl, —OZ¹, —NHOH, —NHOCH₃, —NHCN, and —NZ¹Z² groups, wherein Z¹ and Z², which may be identical or different, are independently chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, and C₇₋₁₂ arylalkyl groups, or Z¹ and Z² join together along with the nitrogen atom to which they are attached to form a ring;
R⁴ is chosen from H, C₁₋₈ alkyl, C₂₋₈ alkenyl, C₂₋₈ alkynyl, C₁₋₈ haloalkyl, C₂₋₈ haloalkenyl, C₂₋₄ haloalkynyl, C₄₋₁₆ cycloalkylalkyl, and C₆₋₁₈ aryl groups;
R⁵ is chosen from —CN, C₁₋₈ alkyl, and C₁₋₄ haloalkyl groups;
M is chosen from

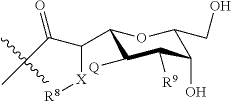

groups
wherein
X is chosen from —O—, —S—, —C—, and —N(R¹⁰)—, wherein R¹⁰ is chosen from H, C₁₋₈ alkyl, C₂₋₈ alkenyl, C₂₋₈ alkynyl, C₁₋₈ haloalkyl, C₂₋₈ haloalkenyl, and C₂₋₈ haloalkynyl groups,
Q is chosen from H, halo, and —OZ³ groups, wherein Z³ is chosen from H and C₁₋₈ alkyl groups,
R⁸ is chosen from H, C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₁₃ heteroaryl, C₇₋₁₉ arylalkyl, and C₂₋₁₄ heteroarylalkyl groups, wherein the C₁₋₈ alkyl, C₂₋₈ alkenyl, C₂₋₈ alkynyl, C₁₋₈ haloalkyl, C₂₋₈ haloalkenyl, C₂₋₈ haloalkynyl, C₄₋₁₆ cycloalkylalkyl, C₆₋₁₈ aryl, C₁₋₁₃ heteroaryl, C₇₋₁₉ arylalkyl, and C₂₋₁₄ heteroarylalkyl groups are optionally substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, —C(═O)NZ⁴Z⁵, and —SO₂Z⁴ groups, wherein Z⁴ and Z⁵, which may be identical or different, are independently chosen from H, C₁₋₈ alkyl, and C₁₋₈ haloalkyl groups, or Z⁴ and Z⁵ join together along with the nitrogen atom to which they are attached to form a ring,
R⁹ is chosen from C₆₋₁₈ aryl and C₁₋₁₃ heteroaryl groups, wherein the C₆₋₁₈ aryl and C₁₋₁₃ heteroaryl groups are optionally substituted with one or more groups independently chosen from R¹¹, C₁₋₈ alkyl, C₁₋₈ haloalkyl, —C(═O)OZ⁶, and —C(═O)NZ⁶Z⁷ groups, wherein R¹¹ is independently chosen from C₆₋₁₈ aryl groups optionally substituted with one or more groups independently chosen from halo, C₁₋₈ alkyl, —OZ⁸, —C(═O)OZ⁸, and —C(═O)NZ⁸Z⁹ groups, wherein Z⁶, Z⁷, Z⁸ and Z⁹, which may be identical or different, are independently chosen from H and C₁₋₈ alkyl groups, or Z⁶ and Z⁷ join together along with the nitrogen atom to which they are attached to form a ring and/or Z⁸ and Z⁹ join together along with the nitrogen atom to which they are attached to form a ring, and
wherein each of Z³, Z⁴, Z⁵, Z⁶, Z⁷, Z⁸, and Z⁹ is optionally substituted with one or more groups independently chosen from halo and —OR¹² groups, wherein R¹² is independently chosen from H and C₁₋₈ alkyl groups; and
L is chosen from linker groups.

In some embodiments, R₁ is chosen from H, C₁₋₄ alkyl, and C₁₋₄ haloalkyl groups. In some embodiments, R₁ is chosen from H, methyl, ethyl, —CH₂F, —CHF₂, —CF₃, —CH₂CH₂F, —CH₂CHF₂, and —CH₂CF₃. In some embodiments, R₁ is H. In some embodiments, R₁ is chosen from methyl and ethyl. In some embodiments, R₁ is methyl. In some embodiments, R₁ is ethyl.

In some embodiments, R¹ is chosen from

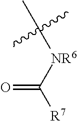

groups.

In some embodiments, R¹ is chosen from

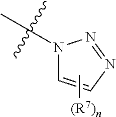

groups.

In some embodiments, R⁶ is chosen from H, C₁₋₈ alkyl, and —C(═O)R⁷ groups. In some embodiments, R⁶ is chosen from H and C₁₋₈ alkyl groups. In some embodiments, R⁶ is chosen from C₁₋₄ alkyl groups. In some embodiments, R⁶ is H.

In some embodiments, each R⁷ is independently chosen from H, C₁₋₈ alkyl, C₆₋₁₈ aryl groups, and C₁₋₁₃ heteroaryl groups. In some embodiments, at least one R⁷ is chosen from C₁₋₈ alkyl groups. In some embodiments, at least one R⁷ is chosen from C₁₋₄ alkyl groups. In some embodiments, at least one R⁷ is chosen from methyl and ethyl. In some embodiments, at least one R⁷ is H. In some embodiments, at least one R⁷ is methyl. In some embodiments, at least one R⁷ is ethyl.

In some embodiments, at least one R⁷ is chosen from

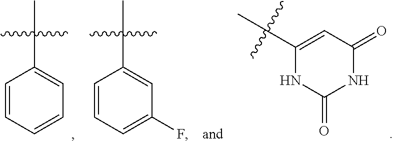

In some embodiments, R¹ is

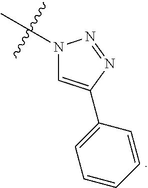 [SMILES: *n1cc(-c2ccccc2)nn1]

In some embodiments, R¹ is

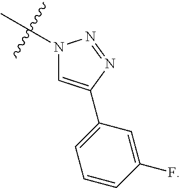 [SMILES: *n1cc(-c2cccc(F)c2)nn1]

In some embodiments, R¹ is

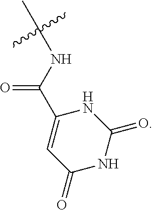 [SMILES: *NC(=O)c1cc(=O)[nH]c(=O)[nH]1]

In some embodiments, R² is chosen from —OH, —OY¹, —OC(═O)Y¹, and —NHC(═O)Y¹ groups, wherein Y¹ is chosen from C₁₋₈ alkyl, C₄₋₁₆ cycloalkylalkyl, C₂₋₁₂ heterocyclyl, C₆₋₁₈ aryl, and C₁₋₁₃ heteroaryl groups. In some embodiments, R² is chosen from —OY¹ groups. In some embodiments, R² is chosen from —OC(═O)Y¹ groups. In some embodiments, R² is chosen from —NHC(═O)Y¹ groups. In some embodiments, R₂ is —OH.

In some embodiments, R² is chosen from

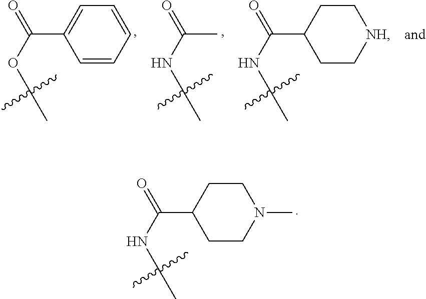

In some embodiments, R² is

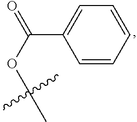 [SMILES: *OC(=O)c1ccccc1]

In some embodiments, R² is

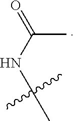 [SMILES: *NC(C)=O]

In some embodiments, R³ is chosen from —C(═O)Y³ groups, wherein Y³ is chosen from —OZ¹, —NHOH, —NHOCH₃, and —NZ¹Z² groups. In some embodiments, R³ is chosen from —C(═O)OZ¹ groups. In some embodiments, R³ is chosen from —C(═O)NZ¹Z² groups. In some embodiments, Z¹ and Z², which may be identical or different, are independently chosen from H, C₁₋₈ alkyl, C₁₋₈ haloalkyl, and C₇₋₁₂ arylalkyl groups. In some embodiments, at least one of Z¹ and Z² is H. In some embodiments, each of Z¹ and Z² is H. In some embodiments, at least one of Z¹ and Z² is methyl. In some embodiments, each of Z¹ and Z² is methyl. In some embodiments, at least one of Z¹ and Z² is ethyl. In some embodiments, each of Z¹ and Z² is ethyl. In some embodiments, Z¹ is H and Z² is methyl. In some embodiments, Z¹ and Z² join together along with the nitrogen atom to which they are attached to form a ring.

In some embodiments, R³ is chosen from

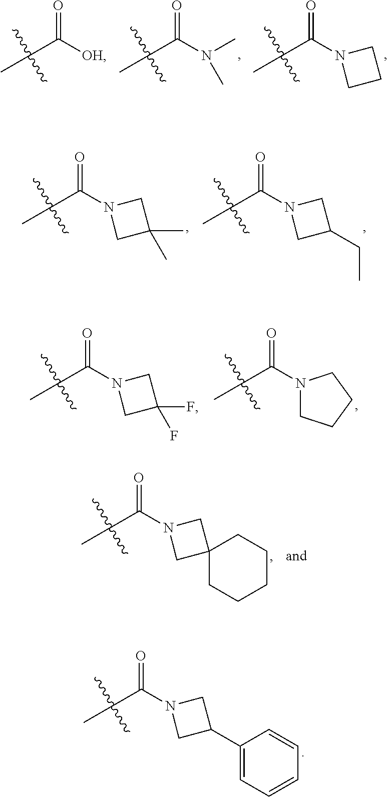

In some embodiments, R³ is chosen from

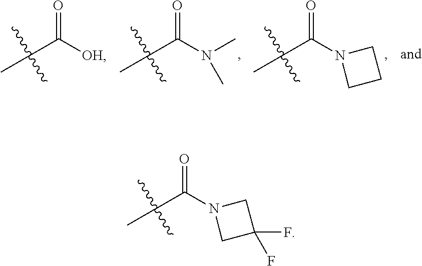

In some embodiments, R³ is

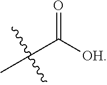 [SMILES: *C(=O)O]

In some embodiments, R³ is

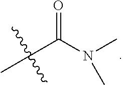 [SMILES: *C(=O)N(C)C]

In some embodiments, R³ is

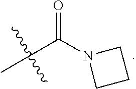 [SMILES: *C(=O)N1CCC1]

In some embodiments, R³ is

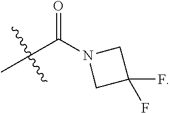 [SMILES: *C(=O)N1CC(F)(F)C1]

In some embodiments, R⁴ is chosen from C₁₋₈ alkyl groups. In some embodiments, R⁴ is chosen from C₁₋₈ haloalkyl groups. In some embodiments, R⁴ is chosen from C₄₋₁₆ cycloalkylalkyl groups. In some embodiments, R⁴ is chosen from C₄₋₈ cycloalkylalkyl groups. In some embodiments, R⁴ is chosen from propyl, cyclopropylmethyl, and cyclohexylmethyl. In some embodiments, R⁴ is propyl. In some embodiments, R⁴ is cyclopropylmethyl. In some embodiments, R⁴ is cyclohexylmethyl.

In some embodiments, R⁵ is chosen from halomethyl groups. In some embodiments, R⁵ is CF₃. In some embodiments, R⁵ is CH₃. In some embodiments, R⁵ is CN.

In some embodiments, M is chosen from

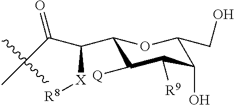

groups.

In some embodiments, M is chosen from

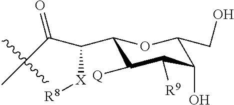

groups.

In some embodiments, X is chosen from —C—, —O—, —S—, and —N(R¹⁰)—, wherein R¹⁰ is chosen from H, C₁₋₈ alkyl, and C₁₋₈ haloalkyl groups. In some embodiments, X is —C—. In some embodiments, X is —O—. In some embodiments, X is —S—. In some embodiments, X is —N(R¹⁰)—. In some embodiments, R¹⁰ is chosen from H, C₁₋₄ alkyl, and C₁₋₄ haloalkyl groups. In some embodiments, R¹⁰ is H. In some embodiments, R¹⁰ is chosen from C₁₋₄ alkyl groups.

In some embodiments, Q is H. In some embodiments. Q is chosen from halo groups. In some embodiments, Q is fluoro. In some embodiments, Q is chosen from —OZ³ groups. In some embodiments, Z³ is chosen from H and C₁₋₄ alkyl groups. In some embodiments, Q is —OH. In some embodiments, Q is —OMe.

In some embodiments, R⁸ is chosen from H, C₁₋₈ alkyl, C₄₋₁₆ cycloalkylalkyl, C₇₋₁₉ arylalkyl, and C₂₋₁₄ heteroarylalkyl groups, wherein the C₁₋₈ alkyl, C₄₋₁₆ cycloalkylalkyl, C₇₋₁₉ arylalkyl, and C₂₋₁₄ heteroarylalkyl groups are optionally substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, —C(═O)NZ⁴Z⁵, and —SO₂Z⁴ groups, wherein Z⁴ and Z₅, which may be identical or different, are independently chosen from H, C₁₋₈ alkyl, and C₁₋₈ haloalkyl groups, or Z⁴ and Z⁵ join together along with the nitrogen atom to which they are attached to form a ring.

In some embodiments, R⁸ is chosen from H, C₁₋₈ alkyl, and C₄₋₁₆ cycloalkylalkyl groups. In some embodiments, R⁸ is chosen from H, C₁₋₄ alkyl, and C₄₋₈ cycloalkylalkyl groups. In some embodiments, R⁸ is H. In some embodiments, R⁸ is chosen from C₁₋₈ alkyl groups. In some embodiments, R⁸ is chosen from C₁₋₄ alkyl groups. In some embodiments, R⁸ is chosen from methyl, ethyl, propyl, and butyl groups. In some embodiments, R⁸ is methyl. In some embodiments, R⁸ is chosen from C₄₋₁₆ cycloalkylalkyl groups. In some embodiments, R⁸ is chosen from C₄₋₈ cycloalkylalkyl groups. In some embodiments, R⁸ is chosen from cyclohexylmethyl and cyclopropylmethyl. In some embodiments, R⁸ is cyclopropylmethyl.

In some embodiments, R⁸ is chosen from C₇₋₁₉ arylalkyl and C₂₋₁₄ heteroarylalkyl groups, wherein the C₇₋₁₉ arylalkyl and C₂₋₁₄ heteroarylalkyl groups are unsubstituted. In some embodiments, R⁸ is chosen from C₇₋₁₉ arylalkyl and C₂₋₁₄ heteroarylalkyl groups, wherein the C₇₋₁₉ arylalkyl and C₂₋₁₄ heteroarylalkyl groups are substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, and —SO₂Z⁴ groups.

In some embodiments, R⁸ is chosen from C₇₋₁₉ arylalkyl groups. In some embodiments, R⁸ is chosen from C₇₋₁₅ arylalkyl groups. In some embodiments, R⁸ is chosen from C₇₋₁₁ arylalkyl groups. In some embodiments, R⁸ is chosen from C₂₋₁₄ heteroarylalkyl groups. In some embodiments, R⁸ is chosen from C₄₋₁₄ heteroarylalkyl groups. In some embodiments, R⁸ is chosen from C₂₋₁₀ heteroarylalkyl groups. In some embodiments, R⁸ is chosen from C₄₋₁₀ heteroarylalkyl groups.

In some embodiments, R⁸ is chosen from C₇₋₁₉ arylalkyl groups, wherein the C₇₋₁₉ arylalkyl groups are unsubstituted. In some embodiments, R⁸ is chosen from C₇₋₁₁ arylalkyl groups, wherein the C₇₋₁₁ arylalkyl groups are unsubstituted.

In some embodiments, R⁸ is chosen from

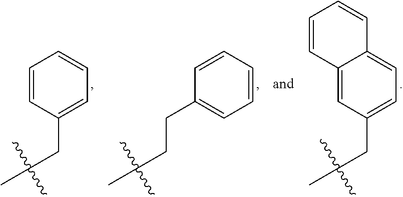 [SMILES: *Cc1ccccc1 ; *CCc1ccccc1 ; *Cc1ccc2ccccc2c1]

In some embodiments, R⁸ is chosen from C₇₋₁₉ arylalkyl groups, wherein the C₇₋₁₉ arylalkyl groups are substituted with one or more groups independently chosen from halo groups. In some embodiments, the halo group is independently chosen from fluoro and chloro. In some embodiments, at least one halo group is fluoro. In some embodiments, at least one halo group is chloro.

In some embodiments, R³ is chosen from

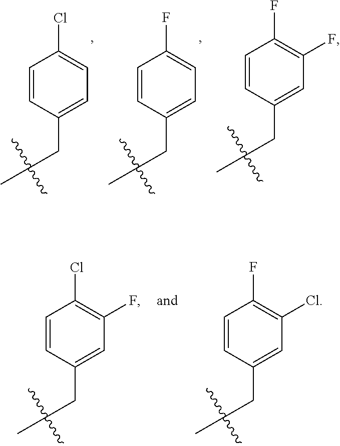 [SMILES: *Cc1ccc(Cl)cc1 ; *Cc1ccc(F)cc1 ; *Cc1ccc(F)c(F)c1 ; *Cc1ccc(Cl)c(F)c1 ; *Cc1ccc(F)c(Cl)c1]

In some embodiments, R⁸ is chosen from C₇₋₁₉ arylalkyl groups, wherein the C₇₋₁₉ arylalkyl groups are substituted with one or more groups independently chosen from C₁₋₈ alkyl, C₁₋₈ hydroxyalkyl, C₁₋₈ haloalkyl, and C₆₋₁₈ aryl groups. In some embodiments, R⁸ is benzyl, wherein the benzyl is substituted with one or more groups independently chosen from C₁₋₈ alkyl, C₁₋₈ hydroxyalkyl, C₁₋₈ haloalkyl, and C₆₋₁₈ aryl groups.

In some embodiments, R⁸ is chosen from

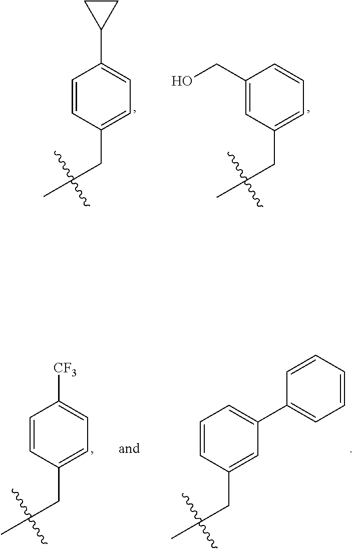 [SMILES: *Cc1ccc(C2CC2)cc1 ; *Cc1cccc(CO)c1 ; *Cc1ccc(C(F)(F)F)cc1 ; *Cc1cccc(-c2ccccc2)c1]

In some embodiments, R⁸ is chosen from C₇₋₁₉ arylalkyl groups, wherein the C₇₋₁₉ arylalkyl groups are substituted with one or more groups independently chosen from —OZ⁴, —C(═O)OZ⁴, and —SO₂Z⁴ groups, wherein Z⁴ is independently chosen from H, C₁₋₈ alkyl, and C₁₋₈ haloalkyl groups. In some embodiments, R⁸ is benzyl, wherein the benzyl is substituted with one or more groups independently chosen from —OZ¹, —C(═O)OZ⁴, and —SO₂Z⁴ groups. In some embodiments, Z⁴ is chosen from H, C₁₋₄ alkyl, and C₁₋₄ haloalkyl groups. In some embodiments, Z⁴ is H. In some embodiments, Z⁴ is chosen from C₁₋₄ alkyl groups. In some embodiments, Z⁴ is methyl. In some embodiments, Z⁴ is chosen from C₁₋₄ haloalkyl groups. In some embodiments, Z⁴ is —CF₃.

In some embodiments, R⁸ is chosen from

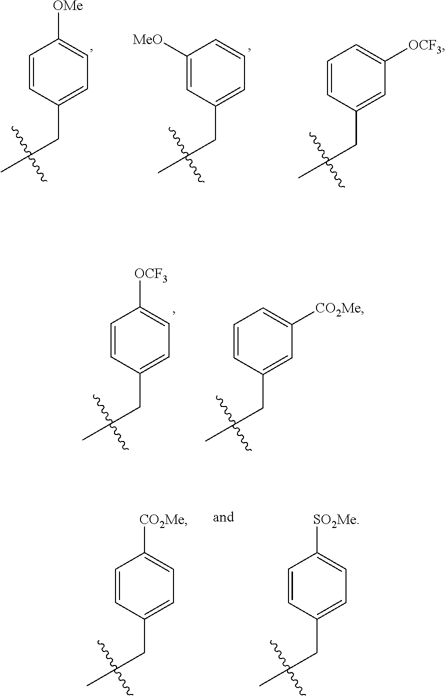

In some embodiments, R⁸ is chosen from C₂₋₁₄ heteroarylalkyl groups, wherein the C₂₋₁₄ heteroarylalkyl groups are unsubstituted. In some embodiments, R⁸ is chosen from C₂₋₁₀ heteroarylalkyl groups, wherein the C₂₋₁₀ heteroarylalkyl groups are unsubstituted. In some embodiments, R⁸ is chosen from C₄₋₁₄ heteroarylalkyl groups, wherein the C₄₋₁₄ heteroarylalkyl groups are unsubstituted. In some embodiments, R⁸ is chosen from C₄₋₁₀ heteroarylalkyl groups, wherein the C₄₋₁₀ heteroarylalkyl groups are unsubstituted.

In some embodiments, R⁸ is chosen from C₂₋₁₄ heteroarylalkyl groups, wherein the C₂₋₁₄ heteroarylalkyl groups are optionally substituted with one or more groups independently chosen from halo, C₁₋₈ alkyl, C₁₋₈ hydroxyalkyl, C₁₋₈ haloalkyl, C₆₋₁₈ aryl, —OZ⁴, —C(═O)OZ⁴, and —SO₂Z⁴ groups, wherein Z⁴ is independently chosen from H and C₁₋₈ alkyl groups. In some embodiments, Z⁴ is chosen from H and methyl. In some embodiments, Z⁴ is H. In some embodiments, Z⁴ is methyl.

In some embodiments, R⁸ is chosen from

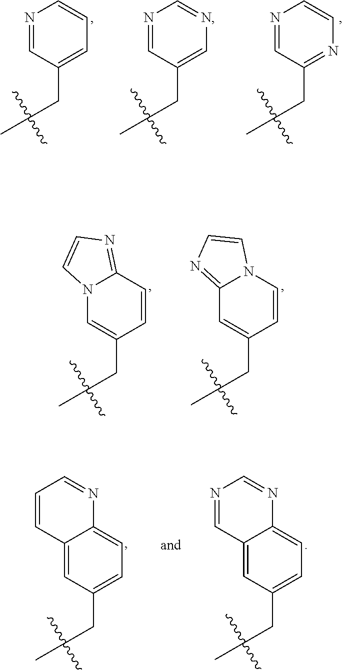

In some embodiments, R⁹ is chosen from C₁₋₁₃ heteroaryl groups optionally substituted with one or more groups independently chosen from R¹¹, C₁₋₈ alkyl, C₁₋₈ haloalkyl, —C(═O)OZ⁶, and —C(═O)NZ⁶Z⁷ groups. In some embodiments, R⁹ is chosen from C₁₋₁₃ heteroaryl groups substituted with one or more groups independently chosen from R¹¹, C₁₋₈ alkyl, C₁₋₈ haloalkyl, —C(═O)OZ⁶, and —C(═O)NZ⁶Z⁷ groups. In some embodiments, R⁹ is chosen from C₂₋₆ heteroaryl groups. In some embodiments, R⁹ is chosen from C₂₋₄ heteroaryl groups.

In some embodiments, R⁹ is chosen from C₁₋₁₃ heteroaryl groups optionally substituted with one or more groups independently chosen from R¹¹. In some embodiments, R⁹ is chosen from C₂₋₄ heteroaryl groups optionally substituted with one or more groups independently chosen from R¹¹. In some embodiments, R⁹ is chosen from C₁₋₁₃ heteroaryl groups substituted with one or more groups independently chosen from R¹¹. In some embodiments, R⁹ is chosen from C₂₋₄ heteroaryl groups substituted with one or more groups independently chosen from R¹¹.

In some embodiments, R¹¹ is chosen from C₆₋₁₈ aryl groups optionally substituted with one or more groups independently chosen from halo groups. In some embodiments, R¹¹ is chosen from phenyl optionally substituted with one or more groups independently chosen from halo groups. In some embodiments, R¹¹ is chosen from C₆₋₁₈ aryl groups substituted with one or more groups independently chosen from halo groups. In some embodiments, R¹¹ is chosen from phenyl substituted with one or more groups independently chosen from halo groups. In some embodiments, at least one halo group is fluoro.

In some embodiments, R⁹ is chosen from

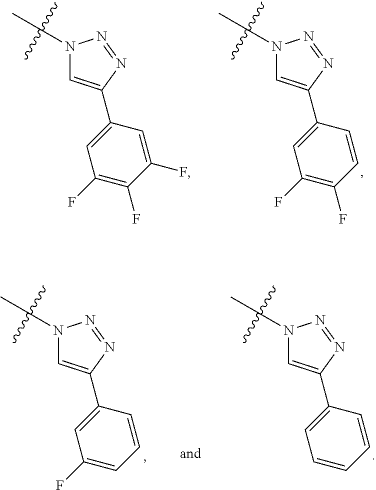 [SMILES: *n1cc(-c2cc(F)c(F)c(F)c2)nn1 ; *n1cc(-c2ccc(F)c(F)c2)nn1 ; *n1cc(-c2cccc(F)c2)nn1 ; *n1cc(-c2ccccc2)nn1]

In some embodiments, R⁹ is

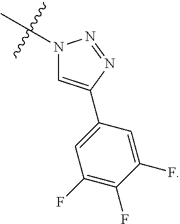 [SMILES: *n1cc(-c2cc(F)c(F)c(F)c2)nn1]

In some embodiments, R⁹ is

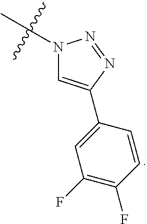 [SMILES: *n1cc(-c2ccc(F)c(F)c2)nn1]

In some embodiments, R⁹ is

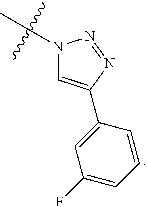 [SMILES: *n1cc(-c2cccc(F)c2)nn1]

In some embodiments, R⁹ is

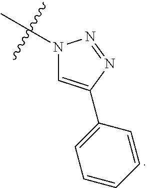 [SMILES: *n1cc(-c2ccccc2)nn1]

In some embodiments, each of Z³, Z⁴, Z⁵, Z⁶, Z⁷, Z⁸, and Z⁹ is unsubstituted. In some embodiments, at least one of Z³, Z⁴, Z⁵, Z⁶, Z⁷, Z⁸, and Z⁹ is substituted. In some embodiments, at least one of Z³, Z⁴, Z⁵, Z⁶, Z⁷, Z⁸, and Z⁹ is substituted with one or more groups independently chosen from halo and —OR¹² groups. In some embodiments, at least one of Z³, Z⁴, Z⁵, Z⁶, Z⁷, Z⁸, and Z⁹ is substituted with one or more groups independently chosen from halo groups. In some embodiments, at least one of Z³, Z⁴, Z⁵, Z⁶, Z⁷, Z⁸, and Z⁹ is substituted with one or more groups independently chosen from —OR¹² groups. In some embodiments, R¹² is H. In some embodiments, R¹² is independently chosen from C₁₋₈ alkyl groups. In some embodiments, R¹² is independently chosen from C₁₋₄ alkyl groups. In some embodiments, R¹² is independently chosen from methyl, ethyl, propyl, and butyl groups. In some embodiments, the halo group is fluoro.

In some embodiments, M is chosen from

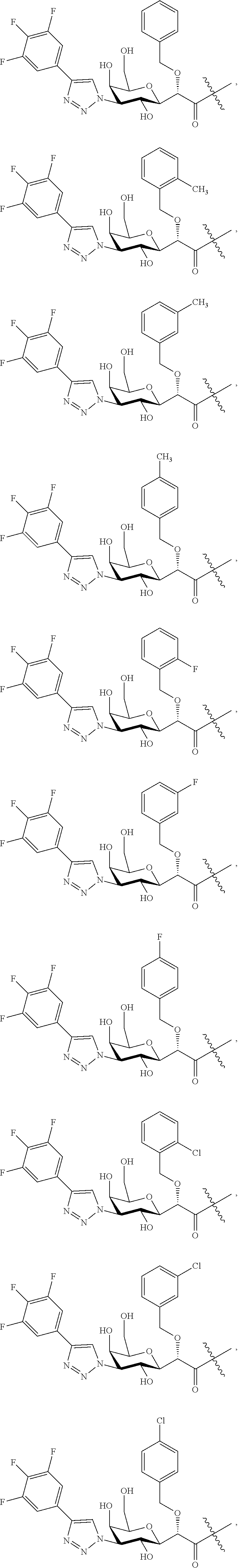

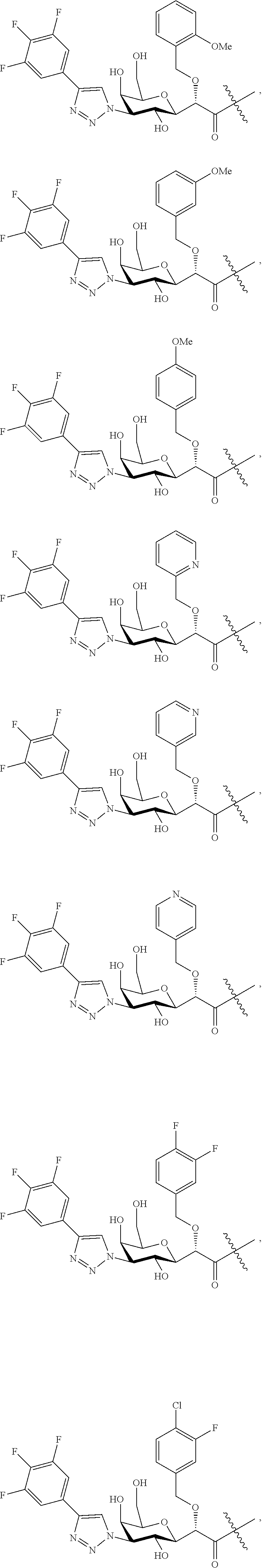

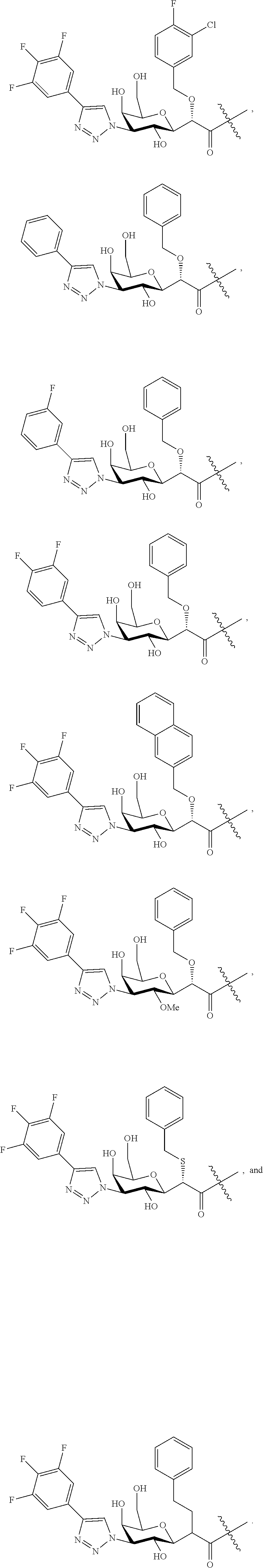

In some embodiments, linker groups may be chosen from groups comprising spacer groups, such spacer groups as, for example, —(CH₂)ₜ— and —O(CH₂)ₜ—, wherein t is chosen from integers ranging from 1 to 20. Other non-limiting examples of spacer groups include carbonyl groups and carbonyl-containing groups such as, for example, amide groups. A non-limiting example of a spacer group is

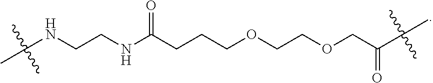 [SMILES: *NCCNC(=O)CCCOCCOCC(*)=O]

In some embodiments, the linker group is chosen from

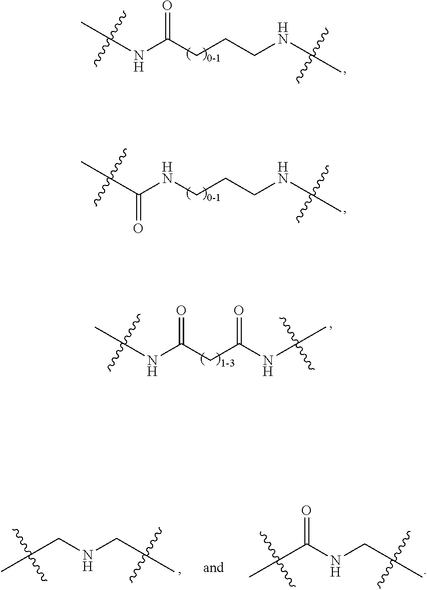

In some embodiments, the linker group is chosen from polyethylene glycols (PEGs), —C(═O)NH(CH₂)ᵥO—, —C(═O)NH(CH₂)ᵥNHC(═O), —C(═O)NHC(═O)(CH₂)NH— and —C(═O)NH(CH₂)ᵥC(═O)NH— groups, wherein v is chosen from integers ranging from 2 to 20. In some embodiments, v is chosen from integers ranging from 2 to 4. In some embodiments, v is 2. In some embodiments, v is 3. In some embodiments, v is 4.

In some embodiments, the linker group is

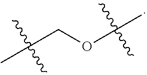

In some embodiments, the linker group is

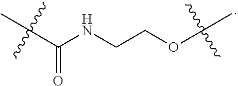 [SMILES: *OCCNC(*)=O]

In some embodiments, the linker group is

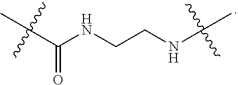 [SMILES: *NCCNC(*)=O]

In some embodiments, the linker group is

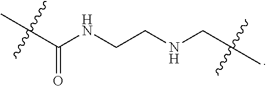 [SMILES: *CNCCNC(*)=O]

In some embodiments, the linker group is

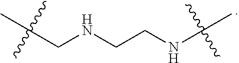 [SMILES: *CNCCN*]

In some embodiments, the linker group is

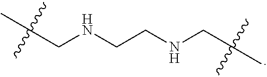 [SMILES: *CNCCNC*]

In some embodiments, the linker group is

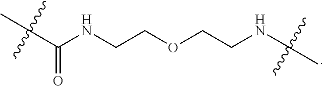 [SMILES: *NCCOCCNC(*)=O]

In some embodiments, the linker group is

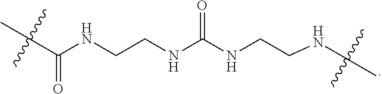 [SMILES: *NCCNC(=O)NCCNC(*)=O]

In some embodiments, the linker group is

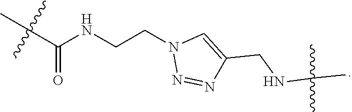 [SMILES: *NCc1cn(CCNC(*)=O)nn1]

In some embodiments, at least one compound is chosen from compounds having the following Formulae:

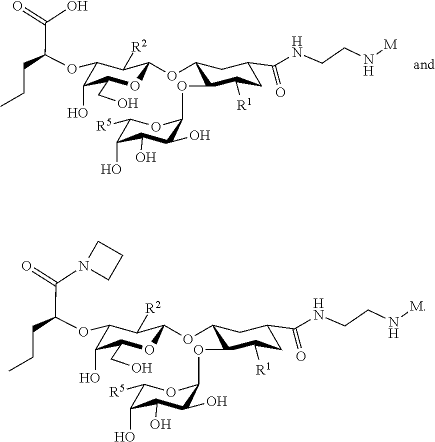

In some embodiments, at least one compound is chosen from compounds having the following Formulae:

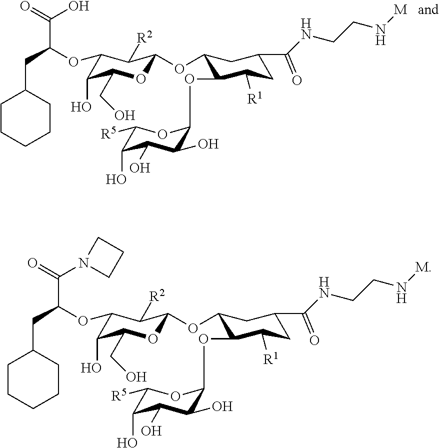

In some embodiments, at least one compound is chosen from compounds having the following Formulae:

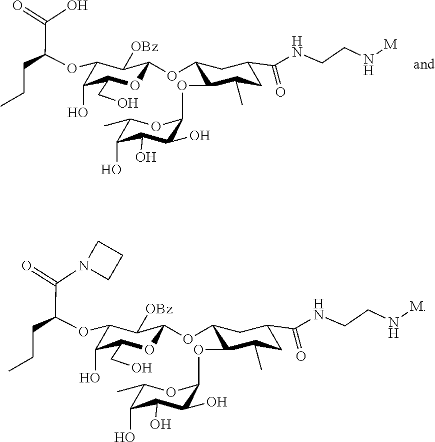

In some embodiments, at least one compound is chosen from compounds having the following Formulae:

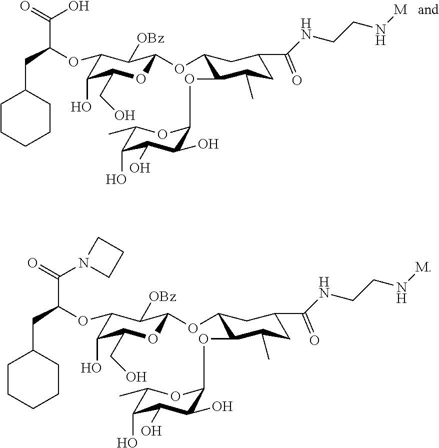

In some embodiments, at least one compound is chosen from compounds having the following Formulae:

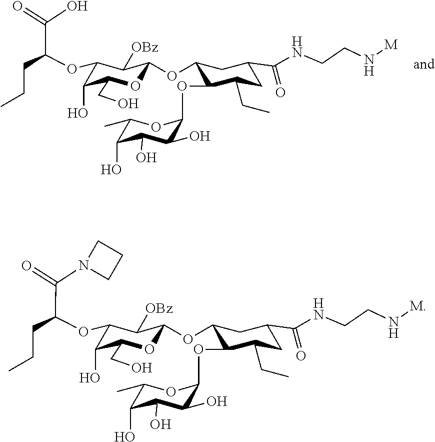

In some embodiments, at least one compound is chosen from compounds having the following Formulae:

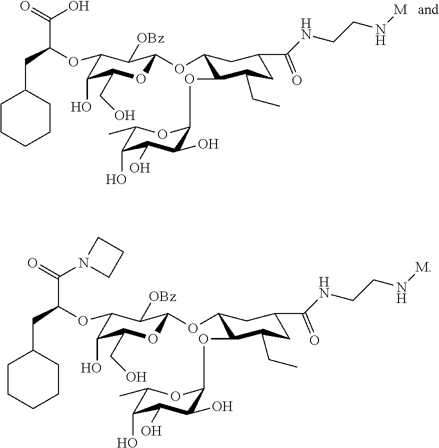

In some embodiments, at least one compound is chosen from compounds having the following Formulae:

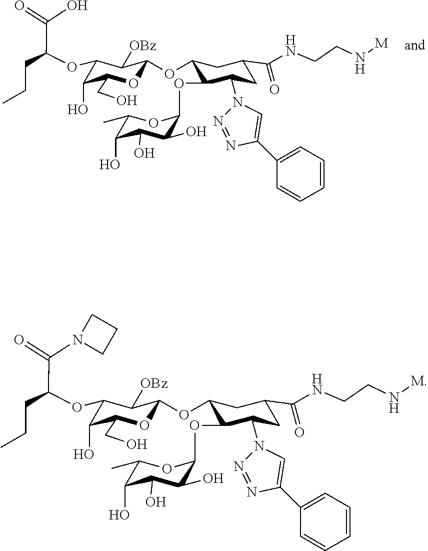

In some embodiments, at least one compound is chosen from compounds having the following Formulae:

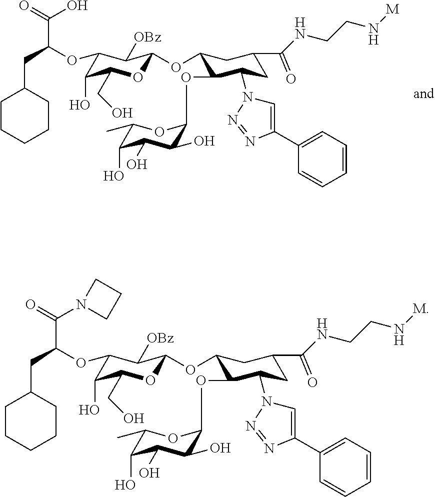

In some embodiments, at least one compound is chosen from compounds having the following Formulae:

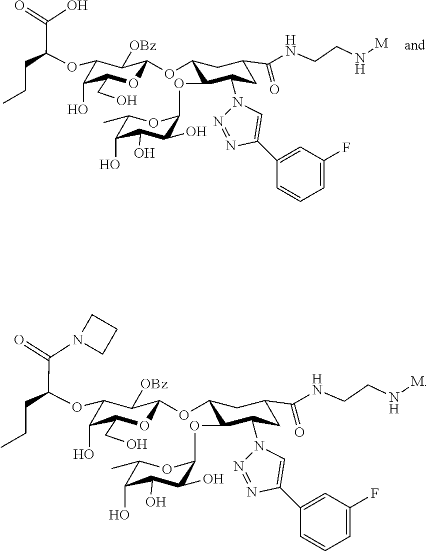

In some embodiments, at least one compound is chosen from compounds having the following Formulae:

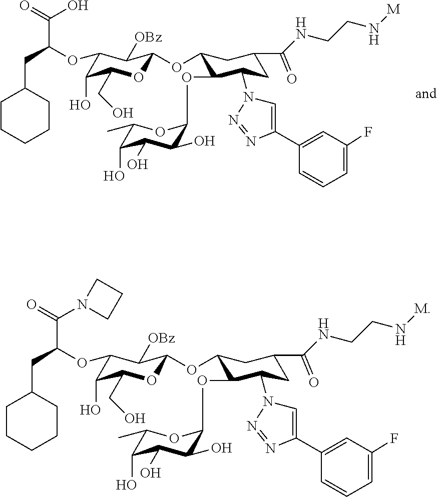

In some embodiments, at least one compound is chosen from compounds having the following Formulae:

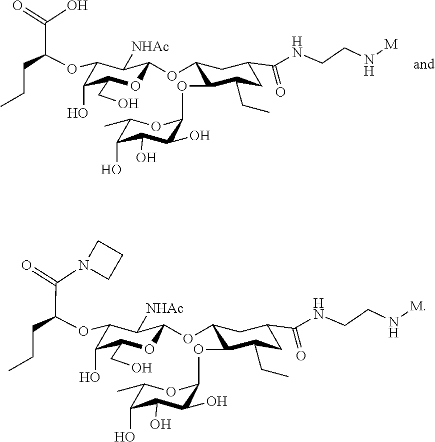

In some embodiments, at least one compound is chosen from compounds having the following Formulae:

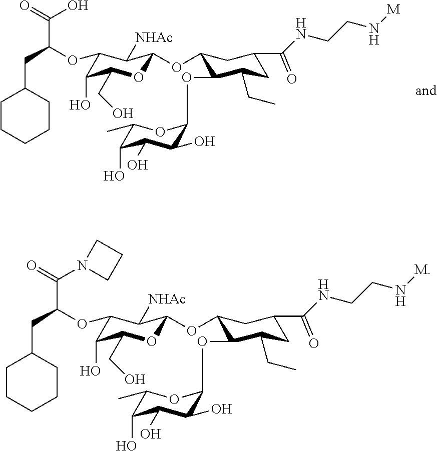

In some embodiments, at least one compound is chosen from compounds having the following Formulae:

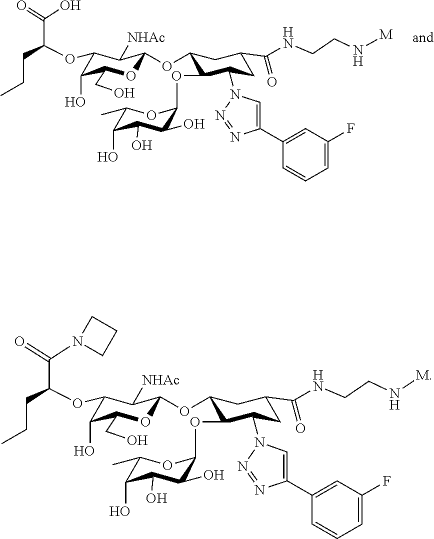

In some embodiments, at least one compound is chosen from compounds having the following Formulae:

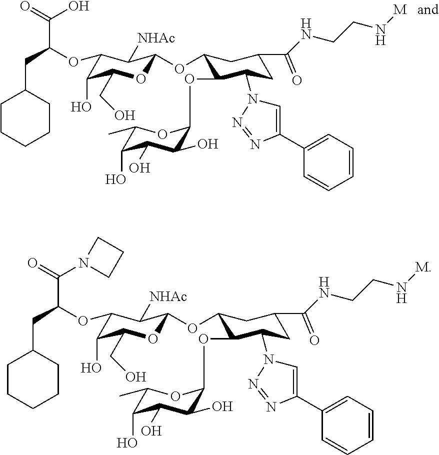

In some embodiments, at least one compound is chosen from compounds having the following Formulae:

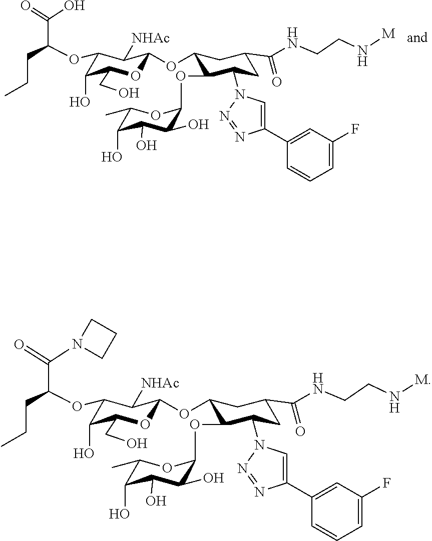

In some embodiments, at least one compound is chosen from compounds having the following Formulae:

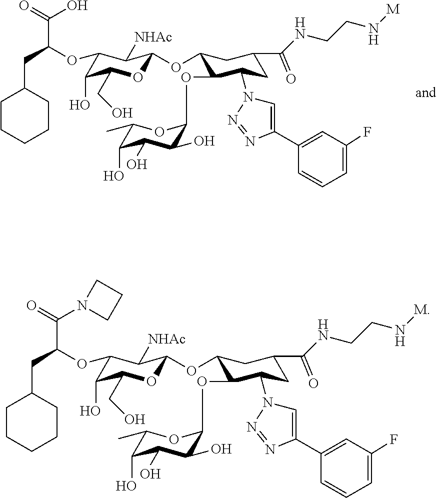

In some embodiments, at least one compound is chosen from compounds having the following Formulae:

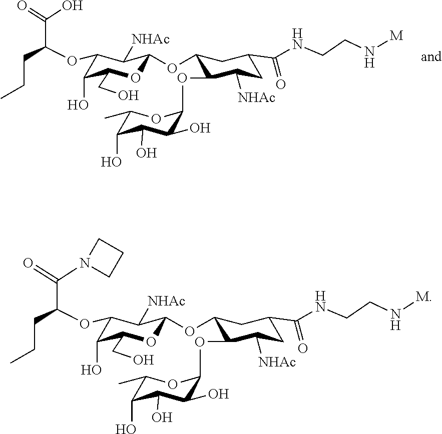

In some embodiments, at least one compound is chosen from compounds having the following Formulae:

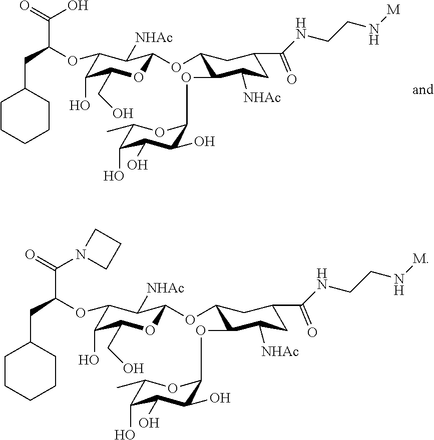

Also provided are pharmaceutical compositions comprising at least one compound of Formula (I). Such pharmaceutical compositions are described in greater detail herein. These compounds and compositions may be used in the methods described herein.

In some embodiments, a method for treating and/or preventing at least one disease, disorder, and/or condition where inhibition of E-selectin mediated functions may be useful is disclosed, the method comprising administering at least one compound of Formula (I) and/or a pharmaceutical composition comprising at least one compound of Formula (I).

In some embodiments, a method for treating and/or preventing at least one disease, disorder, and/or condition where inhibition of galectin-3 mediated functions may be useful is disclosed, the method comprising administering at least one compound of Formula (I) and/or a pharmaceutical composition comprising at least one compound of Formula (I).

In some embodiments, a method for treating and/or preventing at least one disease, disorder, and/or condition where inhibition of both E-selectin and galectin-3 mediated functions may be useful is disclosed, the method comprising administering at least one compound of Formula (I) and/or a pharmaceutical composition comprising at least one compound of Formula (I).

In some embodiments, a method for treating and/or preventing at least one inflammatory disease, disorder, and/or condition in which the adhesion and/or migration of cells occurs in the disease, disorder, and/or condition is disclosed, the method comprising administering at least one compound of Formula (I) and/or a pharmaceutical composition comprising at least one compound of Formula (I).

In some embodiments, a method for regulating the diffusion, compartmentalization, and/or endocytosis of plasma membrane glycoproteins and/or glycolipids is disclosed, the method comprising administering at least one compound of Formula (I) and/or a pharmaceutical composition comprising at least one compound of Formula (I).

In some embodiments, a method for regulating the selection, activation, and/or arrest of T cells, receptor kinase signaling, and/or the functionality of membrane receptors is disclosed, the method comprising administering at least one compound of Formula (I) and/or a pharmaceutical composition comprising at least one compound of Formula (I).

In some embodiments, a method for treating and/or preventing at least one fibrosis is disclosed, the method comprising administering at least one compound of Formula (I) and/or a pharmaceutical composition comprising at least one compound of Formula (I). In some embodiments, the at least one compound of Formula (I) inhibits lattice formation between galectin-3 and glycosylated ligands.

In some embodiments, a method for inhibiting adhesion of a cancer cell that expresses a ligand of E-selectin to an endothelial cell expressing E-selectin on the cell surface of the endothelial cell is disclosed, the method comprising contacting the endothelial cell and at least one compound of Formula (I) and/or a pharmaceutical composition comprising at least one compound of Formula (I) such that the at least one compound of Formula (I) interacts with E-selectin on the endothelial cell, thereby inhibiting binding of the cancer cell to the endothelial cell. In some embodiments, the endothelial cell is present in the bone marrow.

In some embodiment, a method for treating and/or preventing a cancer is disclosed, the method comprising administering to a subject in need thereof an effective amount of at least one compound of Formula (I) and/or a pharmaceutical composition comprising at least one compound of Formula (I). In some embodiments, at least one compound of Formula (I) and/or pharmaceutical composition comprising at least one compound of Formula (I) may be administered in conjunction with (i.e., as an adjunct therapy, which is also called adjunctive therapy) chemotherapy and/or radiotherapy.

The chemotherapy and/or radiotherapy may be referred to as the primary anti-tumor or anti-cancer therapy that is being administered to the subject to treat the particular cancer. In some embodiments, a method for reducing (i.e., inhibiting, diminishing) chemosensitivity and/or radiosensitivity of hematopoietic stem cells (HSC) to the chemotherapeutic drug(s) and/or radiotherapy, respectively, is disclosed, the method comprising administering to a subject in need thereof an effective amount of at least one compound of Formula (I) and/or a pharmaceutical composition comprising at least one compound of Formula (I).

In some embodiments, a method for enhancing (i.e., promoting) survival of hematopoietic stem cells is provided, the method comprising administering to a subject in need thereof at least one compound of Formula (I) and/or a pharmaceutical composition comprising at least one compound of Formula (I).

In some embodiments, a method for decreasing the likelihood of occurrence of metastasis of cancer cells (also called tumor cells herein) in a subject who is in need thereof is disclosed, the method comprising administering an effective amount of at least one compound of Formula (I) and/or a pharmaceutical composition comprising at least one compound of Formula (I).

In some embodiments, a method for treatment and/or prevention of at least one cancer in which the cancer cells may leave the primary site is disclosed, the method comprising administering to a subject in need thereof an effective amount of at least one compound of Formula (I) and/or a pharmaceutical composition comprising at least one compound of Formula (I). A primary site may be, for example, solid tissue (e.g., breast or prostate) or the bloodstream.

In some embodiments, a method for treatment and/or prevention of at least one cancer in which it is desirable to mobilize cancer cells from a site into the bloodstream and/or retain the cancer cells in the bloodstream is disclosed, the method comprising administering to a subject in need thereof an effective amount of at least one compound of Formula (I) and/or a pharmaceutical composition comprising at least one compound of Formula (I).

In some embodiments, a method for decreasing the likelihood of occurrence of infiltration of cancer cells into bone marrow is disclosed, the method comprises administering to a subject in need thereof an effective amount of at least one compound of Formula (I) and/or a pharmaceutical composition comprising at least one compound of Formula (I).

In some embodiments, a method for releasing cells into circulating blood and enhancing retention of the cells in the blood is disclosed, the method comprising administering to a subject in need thereof an effective amount of at least one compound of Formula (I) and/or a pharmaceutical composition comprising at least one compound of Formula (I). In some embodiments, the method further includes collecting the released cells. In some embodiments, collecting the released cells utilizes apheresis. In some embodiments, the released cells are stem cells (e.g., bone marrow progenitor cells). In some embodiments, G-CSF is administered to the individual.

In some embodiments, a method for treating and/or preventing thrombosis is disclosed, the method comprising administering to a subject in need thereof an effective amount of at least one compound of Formula (I) and/or a pharmaceutical composition comprising at least one compound of Formula (I).

In some embodiments, a method for treating and/or preventing mucositis is disclosed, the method comprising administering to a subject in need thereof an effective amount of at least one compound of Formula (I) and/or a pharmaceutical composition comprising at least one compound of Formula (I).

In some embodiments, a method for treating and/or preventing one cardiovascular disease, disorder and/or condition is disclosed, the method comprising administering to a subject in need thereof an effective amount of at least one compound of Formula (I) and/or a pharmaceutical composition comprising at least one compound of Formula (I).

In some embodiments, a method for treatment and/or prevention of atherosclerosis is disclosed, the method comprising administering to a subject in need thereof an effective amount of at least one compound of Formula (I) and/or a pharmaceutical composition comprising at least one compound of Formula (I).

In some embodiments, a method for inhibiting the rejection of transplanted tissue is disclosed, the method comprising administering to a subject in need thereof an effective amount of at least one compound of Formula (I) and/or a pharmaceutical composition comprising at least one compound of Formula (I).

In some embodiments, a method for treatment and/or prevention of pathological angiogenesis is disclosed, the method comprising administering to a subject in need thereof an effective amount of at least one compound of Formula (I) and/or a pharmaceutical composition comprising at least one compound of Formula (I).

In some embodiments, a method for treatment and/or prevention of an epileptic syndrome is disclosed, the method comprising administering to a subject in need thereof at least one compound of Formula (I) and/or a pharmaceutical composition comprising at least one compound of Formula (I).

In some embodiments, a method for treatment and/or prevention of a neurodegenerative disease is disclosed, the method comprising administering to a subject in need thereof an effective amount of at least one compound of Formula (I) and/or a pharmaceutical composition comprising at least one compound of Formula (I).

In some embodiments, a method for treatment and/or prevention of α-synucleinopathies is disclosed, the method comprising administering to a subject in need thereof an effective amount of at least one compound of Formula (I) and/or a pharmaceutical composition comprising at least one compound of Formula (I).

In some embodiments, a method for treatment and prevention of a fibrosing disease or condition is disclosed, the method comprising administering to a subject in need thereof an effective amount of at least one compound of Formula (I) and/or a pharmaceutical composition comprising at least one compound of Formula (I).

In some embodiments, a method for treatment and prevention of sinusoidal obstruction syndrome (SOS) or complications associated therewith is disclosed, the method comprising administering to a subject in need thereof an effective amount of at least one compound of Formula (I) and/or a pharmaceutical composition comprising at least one compound of Formula (I).

In some embodiments, a compound of Formula (I) and/or a pharmaceutical composition comprising at least one compound of Formula (I) may be used for the preparation and/or manufacture of a medicament for use in treating and/or preventing at least one of the diseases, disorders, and/or conditions described herein.

Whenever a term in the specification is identified as a range (e.g., C₁₋₄ alkyl) or “ranging from”, the range independently discloses and includes each element of the range. As a non-limiting example, C₁₋₄ alkyl groups includes, independently, C₁ alkyl groups, C₂ alkyl groups, C₃ alkyl groups, and C₄ alkyl groups. As another non-limiting example, “n is an integer ranging from 0 to 2” includes, independently, 0, 1, and 2.

The terms “at least one” and “one or more” are intended to be synonymous and to refer to no less than one but possibly more, such as one, two, three, etc. For example, the term “at least one C₁₋₄ alkyl group” refers to one or more C₁₋₄ alkyl groups, such as one C₁₋₄ alkyl group, two C₁₋₄ alkyl groups, etc.

The term “alkyl” includes saturated straight, branched, and cyclic (also identified as cycloalkyl), primary, secondary, and tertiary hydrocarbon groups. Non-limiting examples of alkyl groups include methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless stated otherwise specifically in the specification, an alkyl group may be optionally substituted.

The term “alkenyl” includes straight, branched, and cyclic hydrocarbon groups comprising at least one double bond. The double bond of an alkenyl group can be unconjugated or conjugated with another unsaturated group. Non-limiting examples of alkenyl groups include vinyl, allyl, butenyl, pentenyl, hexenyl, butadienyl, pentadienyl, hexadienyl, 2-ethylhexenyl, and cyclopent-1-en-1-yl. Unless stated otherwise specifically in the specification, an alkenyl group may be optionally substituted.

The term “alkynyl” includes straight and branched hydrocarbon groups comprising at least one triple bond. The triple bond of an alkynyl group can be unconjugated or conjugated with another unsaturated group. Non-limiting examples of alkynyl groups include ethynyl, propynyl, butynyl, pentynyl, and hexynyl. Unless stated otherwise specifically in the specification, an alkynyl group may be optionally substituted.

The term “aryl” includes hydrocarbon ring system groups comprising at least 6 carbon atoms and at least one aromatic ring. The aryl group may be a monocyclic, bicyclic, tricyclic or tetracyclic ring system, which may include fused or bridged ring systems. Non-limiting examples of aryl groups include aryl groups derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group may be optionally substituted.

The terms “E-selectin antagonist” and “E-selectin inhibitor” are used interchangeably herein, and include inhibitors of E-selectin only, as well as inhibitors of E-selectin and either P-selectin or L-selectin, and inhibitors of E-selectin, P-selectin, and L-selectin.

The terms “galectin-3 antagonist,” “galectin-3 inhibitor,” and “inhibitors of galectin-3” are used interchangeably herein, and include inhibitors of galectin-3 only, as well as inhibitors of galectin-3 and one or more other galectin, such as galectin-1, galectin-2, galectin-4, galectin-5, galectin-6, galectin-7, galectin-8, galectin-9, galectin-10, galectin-11, and galectin-12.

The term “glycomimetic” includes any naturally occurring or non-naturally occurring carbohydrate compound in which at least one substituent has been replaced, or at least one ring has been modified (e.g., substitution of carbon for a ring oxygen), to yield a compound that is not fully carbohydrate.

The term “halo” or “halogen” includes fluoro, chloro, bromo, and iodo.

The term “haloalkyl” includes alkyl groups, as defined herein, substituted by at least one halogen, as defined herein. Non-limiting examples of haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl. A “fluoroalkyl” is a haloalkyl wherein at least one halogen is fluoro. Unless stated otherwise specifically in the specification, a haloalkyl group may be optionally substituted.

The term “haloalkenyl” includes alkenyl groups, as defined herein, substituted by at least one halogen, as defined herein. Non-limiting examples of haloalkenyl groups include fluoroethenyl, 1,2-difluoroethenyl, 3-bromo-2-fluoropropenyl, and 1,2-dibromoethenyl. A “fluoroalkenyl” is a haloalkenyl substituted with at least one fluoro group. Unless stated otherwise specifically in the specification, a haloalkenyl group may be optionally substituted.

The term “haloalkynyl” includes alkynyl groups, as defined herein, substituted by at least one halogen, as defined herein. Non-limiting examples include fluoroethynyl, 1,2-difluoroethynyl, 3-bromo-2-fluoropropynyl, and 1,2-dibromoethynyl. A “fluoroalkynyl” is a haloalkynyl wherein at least one halogen is fluoro. Unless stated otherwise specifically in the specification, a haloalkynyl group may be optionally substituted.

The term “heterocyclyl” or “heterocyclic ring” includes 3- to 24-membered saturated or partially unsaturated non-aromatic ring groups comprising 2 to 23 ring carbon atoms and 1 to 8 ring heteroatom(s) each independently chosen from N, O, and S. Unless stated otherwise specifically in the specification, the heterocyclyl groups may be monocyclic, bicyclic, tricyclic or tetracyclic ring systems, which may include fused or bridged ring systems, and may be partially or fully saturated; any nitrogen, carbon or sulfur atom(s) in the heterocyclyl group may be optionally oxidized; any nitrogen atom in the heterocyclyl group may be optionally quaternized; and the heterocyclyl group Non-limiting examples of heterocyclic ring include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

The term “heteroaryl” includes 5- to 14-membered ring groups comprising 1 to 13 ring carbon atoms and 1 to 6 ring heteroatom(s) each independently chosen from N, O, and S, and at least one aromatic ring. Unless stated otherwise specifically in the specification, the heteroaryl group may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Non-limiting examples include azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

Unless stated otherwise specifically in the specification, substituents may be optionally substituted.

The term “pharmaceutically acceptable salts” includes both acid and base addition salts. Non-limiting examples of pharmaceutically acceptable acid addition salts include chlorides, bromides, sulfates, nitrates, phosphates, sulfonates, methane sulfonates, formates, tartrates, maleates, citrates, benzoates, salicylates, and ascorbates. Non-limiting examples of pharmaceutically acceptable base addition salts include sodium, potassium, lithium, ammonium (substituted and unsubstituted), calcium, magnesium, iron, zinc, copper, manganese, and aluminum salts. Pharmaceutically acceptable salts may, for example, be obtained using standard procedures well known in the field of pharmaceuticals.

The term “prodrug” includes compounds that may be converted, for example, under physiological conditions or by solvolysis, to a biologically active compound described herein. Thus, the term “prodrug” includes metabolic precursors of compounds described herein that are pharmaceutically acceptable. A discussion of prodrugs can be found, for example, in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” also includes covalently bonded carriers that release the active compound(s) as described herein in vivo when such prodrug is administered to a subject. Non-limiting examples of prodrugs include ester and amide derivatives of hydroxy, carboxy, mercapto and amino functional groups in the compounds described herein.

The term “substituted” includes the situation where, in any of the above groups, at least one hydrogen atom is replaced by a non-hydrogen atom such as, for example, a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also includes the situation where, in any of the above groups, at least one hydrogen atom is replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles.

The present disclosure includes within its scope all the possible geometric isomers, e.g., Z and E isomers (cis and trans isomers), of the compounds as well as all the possible optical isomers, e.g., diastereomers and enantiomers, of the compounds. Furthermore, the present disclosure includes in its scope both the individual isomers and any mixtures thereof, e.g., racemic mixtures. The individual isomers may be obtained using the corresponding isomeric forms of the starting material or they may be separated after the preparation of the end compound according to conventional separation methods. For the separation of optical isomers, e.g., enantiomers, from the mixture thereof conventional resolution methods, e.g., fractional crystallization, may be used.

The present disclosure includes within its scope all possible tautomers. Furthermore, the present disclosure includes in its scope both the individual tautomers and any mixtures thereof.

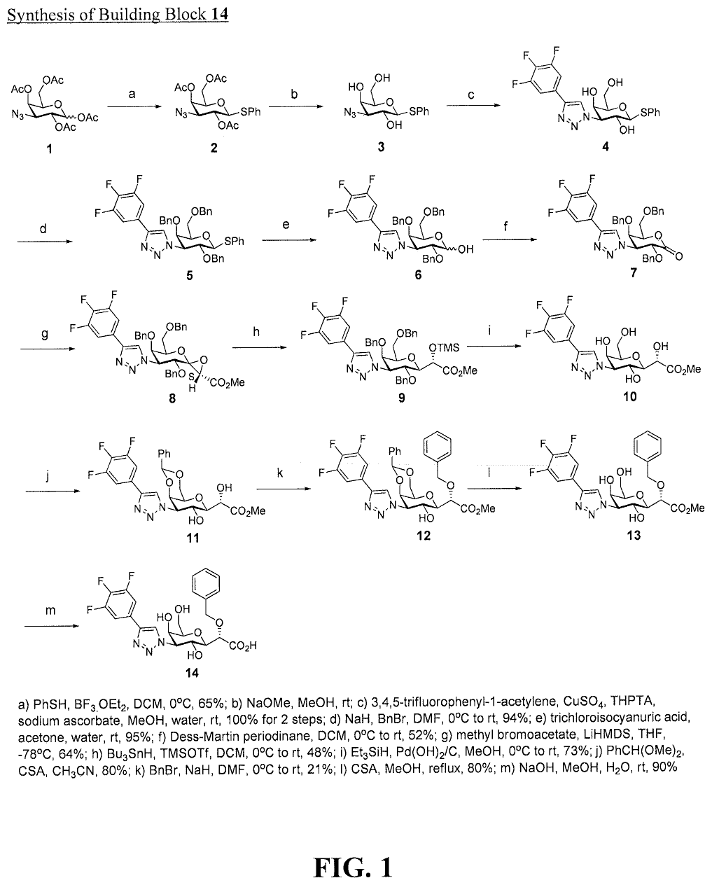

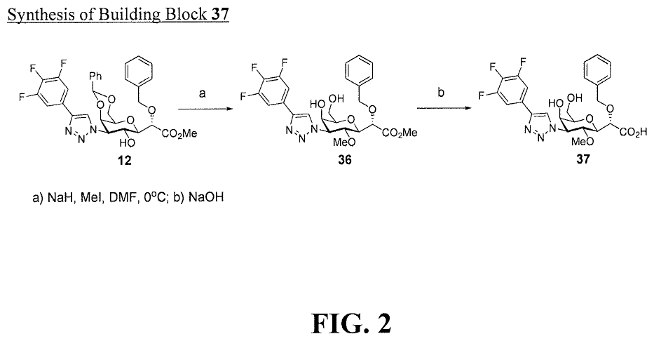

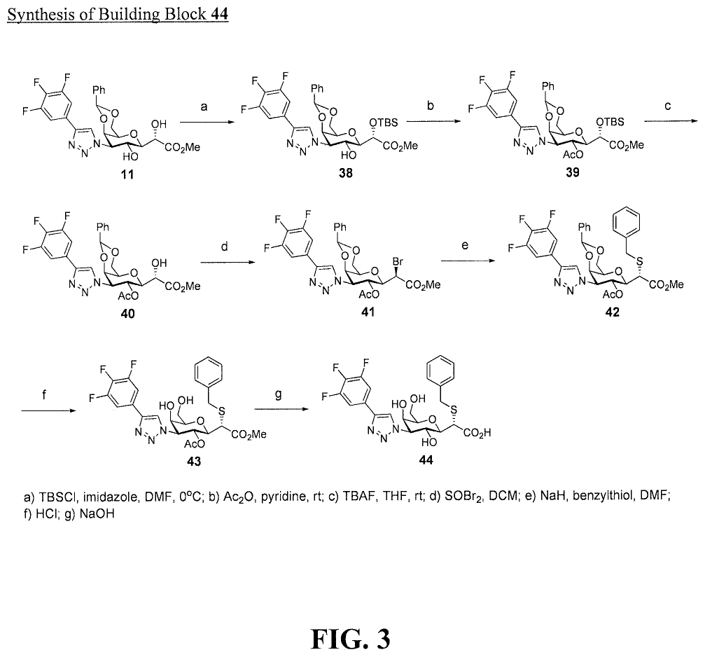

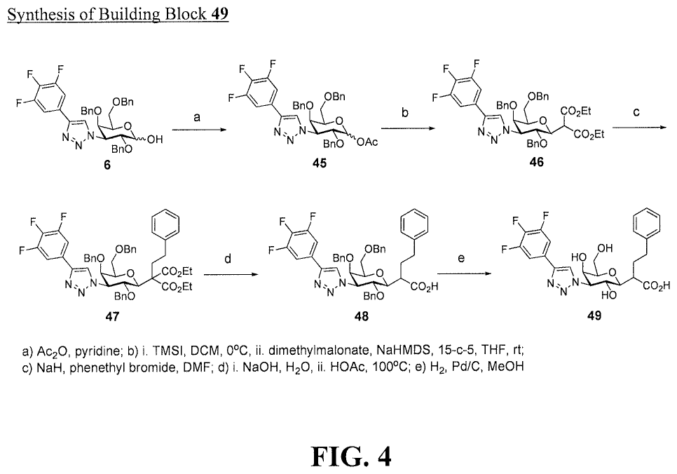

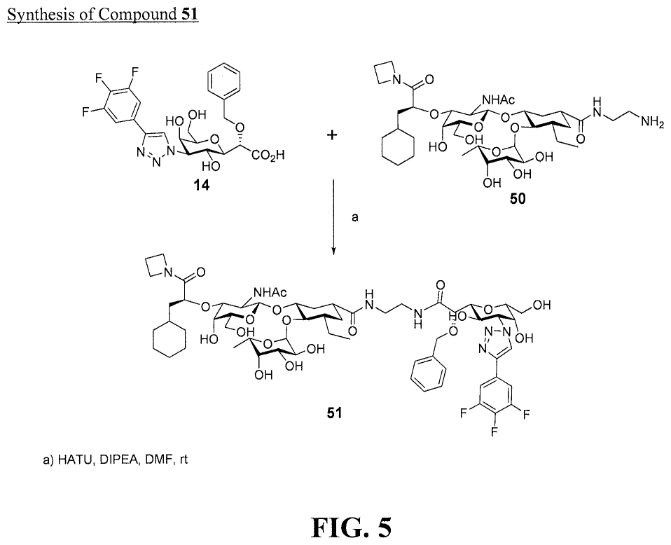

Compounds of Formula (I) may be prepared as shown in FIGS. 1 through 8. It is understood that one of ordinary skill in the art may be able to make these compounds by similar methods or by combining other methods known to one of ordinary skill in the art. It is also understood that one of ordinary skill in the art would be able to make other compounds of Formula (I) not specifically illustrated herein by using appropriate starting components and modifying the parameters of the synthesis as needed. In general, starting components may be obtained from sources such as Sigma Aldrich, Alfa Aesar, Maybridge, Matrix Scientific, TCI, and Fluorochem USA, etc. and/or synthesized according to sources known to those of ordinary skill in the art (see, for example, Advanced Organic Chemistry: Reactions, Mechanisms, and Structure, 5th edition (Wiley, December 2000)) and/or prepared as described herein.

It will also be appreciated by those skilled in the art that in the processes described herein the functional groups of intermediate compounds may need to be protected by suitable protecting groups, even if not specifically described. Such functional groups include hydroxy, amino, mercapto, and carboxylic acid. Suitable protecting groups for hydroxy include but are not limited to trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include but are not limited to t-butoxycarbonyl, benzyloxycarbonyl, and the like. Suitable protecting groups for mercapto include but are not limited to —C(O)R″ (where R″ is alkyl, aryl or arylalkyl), p-methoxybenzyl, trityl and the like. Suitable protecting groups for carboxylic acid include but are not limited to alkyl, aryl or arylalkyl esters. Protecting groups may be added or removed in accordance with standard techniques, which are known to one skilled in the art and as described herein. The use of protecting groups is described in detail in Green, T.W. and P.G.M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

Analogous reactants to those described herein may be identified through the indices of known chemicals prepared by the Chemical Abstract Service of the American Chemical Society, which are available in most public and university libraries, as well as through on-line databases (the American Chemical Society, Washington, D.C., may be contacted for more details). Chemicals that are known but not commercially available in catalogs may be prepared by custom chemical synthesis houses, where many of the standard chemical supply houses (e.g., those listed above) provide custom synthesis services. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002.

Methods known to one of ordinary skill in the art may be identified through various reference books, articles, and databases. Suitable reference books and treatise that detail the synthesis of reactants useful in the preparation of compounds of the present disclosure, or provide references to articles that describe the preparation, include for example, “Synthetic Organic Chemistry,” John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L Gilchrist, “Heterocyclic Chemistry”, 2nd Ed., John Wiley & Sons, New York, 1992; J. March, “Advanced Organic Chemistry: Reactions, Mechanisms and Structure,” 4th Ed., Wiley-Interscience, New York, 1992. Additional suitable reference books and treatise that detail the synthesis of reactants useful in the preparation of compounds of the present disclosure, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Quin, L. D. et al. “A Guide to Organophosphorus Chemistry” (2000) Wiley-Interscience, ISBN: 0-471-31824-8; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Biological activity of a compound described herein may be determined, for example, by performing at least one in vitro and/or in vivo study routinely practiced in the art and described herein or in the art. In vitro assays include without limitation binding assays, immunoassays, competitive binding assays, and cell-based activity assays.

An inhibition assay may be used to screen for antagonists of E-selectin. For example, an assay may be performed to characterize the capability of a compound described herein to inhibit (i.e., reduce, block, decrease, or prevent in a statistically or biologically significant manner) interaction of E-selectin with sLe^{a} or sLe^{x}. The inhibition assay may be a competitive binding assay, which allows the determination of IC₅₀ values. By way of example, E-selectin/Ig chimera may be immobilized onto a matrix (e.g., a multi-well plate, which may be made from a polymer, such as polystyrene; a test tube, and the like); a composition may be added to reduce nonspecific binding (e.g., a composition comprising non-fat dried milk or bovine serum albumin or other blocking buffer routinely used by a person skilled in the art); the immobilized E-selectin may be contacted with the candidate compound in the presence of sLe^{a} comprising a reporter group under conditions and for a time sufficient to permit sLe^{a} to bind to the immobilized E-selectin; the immobilized E-selectin may be washed; and the amount of sLe^{a} bound to immobilized E-selectin may be detected. Variations of such steps can be readily and routinely accomplished by a person of ordinary skill in the art.

An inhibition assay may be used to screen for antagonists of galectin-3. For example, an assay may be performed to characterize the capability of a compound described herein to inhibit interaction of galectin-3 with a Galβ1-3GlcNAc carbohydrate structure. The inhibition assay may be a competitive binding assay, which allows the determination of IC₅₀ values. By way of example, a Galβ1-3GlcNAc polymer may be immobilized onto a matrix; a composition may be added to reduce nonspecific binding; the immobilized Galβ1-3GlcNAc polymer may be contacted with the candidate compound in the presence of galectin-3 group under conditions and for a time sufficient to permit galectin-3 to bind to the immobilized Galβ1-3GlcNAc polymer; the immobilized Galβ1-3GlcNAc polymer may be washed; and the amount of galectin-3 bound to the immobilized Galβ1-3GlcNAc polymer may be detected. Variations of such steps can be readily and routinely accomplished by a person of ordinary skill in the art.

Conditions for a particular assay include temperature, buffers (including salts, cations, media), and other components that maintain the integrity of any cell used in the assay and the compound, which a person of ordinary skill in the art will be familiar and/or which can be readily determined. A person of ordinary skill in the art also readily appreciates that appropriate controls can be designed and included when performing the in vitro methods and in vivo methods described herein.

The source of a compound that is characterized by at least one assay and techniques described herein and in the art may be a biological sample that is obtained from a subject who has been treated with the compound. The cells that may be used in the assay may also be provided in a biological sample. A “biological sample” may include a sample from a subject, and may be a blood sample (from which serum or plasma may be prepared), a biopsy specimen, one or more body fluids (e.g., lung lavage, ascites, mucosal washings, synovial fluid, urine), bone marrow, lymph nodes, tissue explant, organ culture, or any other tissue or cell preparation from the subject or a biological source. A biological sample may further include a tissue or cell preparation in which the morphological integrity or physical state has been disrupted, for example, by dissection, dissociation, solubilization, fractionation, homogenization, biochemical or chemical extraction, pulverization, lyophilization, sonication, or any other means for processing a sample derived from a subject or biological source. In some embodiments, the subject or biological source may be a human or non-human animal, a primary cell culture (e.g., immune cells), or culture adapted cell line, including but not limited to, genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, and the like.

As described herein, methods for characterizing E-selectin and/or galectin-3 antagonists include animal model studies. Non-limiting examples of animal models for liquid cancers used in the art include multiple myeloma (see, e.g., DeWeerdt, Nature 480:S38-S39 (15 Dec. 2011) doi:10.1038/480S38a; Published online 14 Dec. 2011; Mitsiades et al., Clin. Cancer Res. 2009 15:1210021 (2009)); acute myeloid leukemia (AML) (Zuber et al., Genes Dev. 2009 Apr. 1; 23(7): 877-889). Animal models for acute lymphoblastic leukemia (ALL) have been used by persons of ordinary skill in the art for more than two decades. Numerous exemplary animal models for solid tumor cancers are routinely used and are well known to persons of ordinary skill in the art.

The compounds of the present disclosure and the pharmaceutical compositions comprising at least one of such compounds may be useful in methods for treating and/or preventing a disease or disorder that is treatable by inhibiting at least one activity of E-selectin and/or galectin-3 (and/or inhibiting binding of E-selectin and/or galectin-3 to ligand(s), which in turn inhibits a biological activity).

The compounds of the present disclosure and pharmaceutical compositions comprising at least one such compound may be useful in methods for treating and/or preventing at least one inflammatory disease. Inflammation comprises reaction of vascularized living tissue to injury. By way of example, although E-selectin and galectin-3 mediated cell adhesion may be important to the body's anti-infective immune response, in other circumstances, E-selectin and/or galectin-3 mediated cell adhesion may be undesirable or excessive, resulting in tissue damage and/or scarring instead of repair. For example, many pathologies (such as autoimmune and inflammatory diseases, shock and reperfusion injuries) involve abnormal adhesion of white blood cells. Therefore, inflammation affects blood vessels and adjacent tissues in response to an injury or abnormal stimulation by a physical, chemical, or biological agent. Examples of inflammatory diseases, disorders, or conditions include, without limitation, dermatitis, chronic eczema, psoriasis, multiple sclerosis, rheumatoid arthritis, systemic lupus erythematosus, graft versus host disease, sepsis, diabetes, atherosclerosis, Sjogren's syndrome, progressive systemic sclerosis, scleroderma, acute coronary syndrome, ischemic reperfusion, Crohn's disease, inflammatory bowel disease, endometriosis, glomerulonephritis, myasthenia gravis, idiopathic pulmonary fibrosis, asthma, allergic reaction, acute respiratory distress syndrome (ARDS) or other acute leukocyte-mediated lung injury, vasculitis, or inflammatory autoimmune myositis. Other diseases and disorders for which the compounds described herein may be useful for treating and/or preventing include hyperactive coronary circulation, microbial infection, cancer metastasis, thrombosis, wounds, burns, spinal cord damage, digestive tract mucous membrane disorders (e.g., gastritis, ulcers), osteoporosis, osteoarthritis, septic shock, traumatic shock, stroke, nephritis, atopic dermatitis, frostbite injury, adult dyspnoea syndrome, ulcerative colitis, diabetes and reperfusion injury following ischemic episodes, prevention of restenosis associated with vascular stenting, and for undesirable angiogenesis, for example, angiogenesis associated with tumor growth.

As discussed in detail herein, a disease or disorder to be treated or prevented is a cancer and related metastasis and includes cancers that comprise solid tumor(s) and cancers that comprise liquid tumor(s). The compounds of the present disclosure and pharmaceutical compositions comprising at least one such compound may be useful in methods for preventing and/or treating cancer. In some embodiments, the at least one compound may be used for treating and/or preventing metastasis and/or for inhibiting (slowing, retarding, or preventing) metastasis of cancer cells.

In some embodiments, at least one compound of Formula (I) and/or a pharmaceutical composition comprising at least one compound of Formula (I) is administered to a cancer patient in remission. In some embodiments, the at least one compound of Formula (I) and/or a pharmaceutical composition comprising at least one compound of Formula (I) is administered as a cancer vaccine to stimulate marrow infiltrating lymphocytes (“MILs”) in a cancer patient or cancer survivor to prevent relapse.

In some embodiments, a method of treating cancer and/or preventing a cancer relapse is disclosed, wherein the method comprises administering to a patient in need thereof an effective amount of at least one compound of Formula (I) and/or a pharmaceutical composition comprising at least one compound of Formula (I), wherein the amount of compound of Formula (I) administered is sufficient to mobilize MILs of the patient into the peripheral blood.

In some embodiments, a method of treating cancer and/or preventing a cancer relapse is provided comprising administering to a donor patient at least one compound of Formula (I) and/or a pharmaceutical composition comprising at least one compound of Formula (I) in an amount of sufficient to mobilize MILs of the patient out of the marrow (e.g., into the peripheral blood), recovering MILS (e.g., recovering them from the peripheral blood), and transplanting at least a portion of the MIL cell population to the donor patient or another patient. In some embodiments, the MIL cell population is expanded ex vivo before transplantation.

In some embodiments, a method of preventing cancer is provided comprising administering to a donor patient at least one compound of Formula (I) and/or a pharmaceutical composition comprising at least one compound of Formula (I) in an amount sufficient to mobilize MILs of the patient out of the bone marrow (e.g., into the peripheral blood), recovering MILs (e.g., recovering them from the peripheral blood), and transplanting at least a portion of MIL cell population to a subject (e.g., a non-cancer patient, a patient suffering from a different form or type of cancer than the donor patient, etc.). In some embodiments, the MIL cell population is expanded ex vivo before transplantation.

In some embodiments, the compounds of present disclosure and pharmaceutical compositions comprising at least one such compound may be used for decreasing (i.e., reducing) the likelihood of occurrence of metastasis of cancer cells in an individual (i.e., subject, patient) who is in need thereof. The compounds of the present disclosure and compositions comprising at least one such compound may be used for decreasing (i.e., reducing) the likelihood of occurrence of infiltration of cancer cells into bone marrow in an individual who is in need thereof. The individuals (or subjects) in need of such treatments include subjects who have been diagnosed with a cancer, which includes cancers that comprise solid tumor(s) and cancers that comprise liquid tumor(s).

Non-limiting examples of cancers include colorectal cancers, liver cancers, gastric cancers, lung cancers, brain cancers, kidney cancers, bladder cancers, thyroid cancers, prostate cancers, ovarian cancers, cervical cancers, uterine cancers, endometrial cancers, melanomas, breast cancers, and pancreatic cancers. Liquid tumors can occur in the blood, bone marrow, the soft, sponge-like tissue in the center of most bones, and lymph nodes and include leukemias (e.g., AML, ALL, CLL, and CML), lymphomas, and myelomas (e.g., multiple myeloma). Lymphomas include Hodgkin lymphoma, which is marked by the presence of a type of cell called the Reed-Sternberg cell, and non-Hodgkin lymphomas, which includes a large, diverse group of cancers of immune system cells. Non-Hodgkin lymphomas can be further divided into cancers that have an indolent (slow-growing) course and those that have an aggressive (fast-growing) course, and which subtypes respond to treatment differently.

The compounds of the present disclosure and pharmaceutical compositions comprising at least one such compound may be administered as an adjunct therapy to chemotherapy and/or radiotherapy, which is/are being delivered to the subject as primary therapy for treating the cancer. The chemotherapy and/or radiotherapy that may be administered depend upon several factors including the type of cancer, location of the tumor(s), stage of the cancer, age and gender and general health status of the subject. A person of ordinary skill in the medical art can readily determine the appropriate chemotherapy regimen and/or radiotherapy regimen for the subject in need. The person of ordinary skill in the medical art can also determine, with the aid of preclinical and clinical studies, when the compound of the present disclosure or pharmaceutical composition comprising at least one such compound should be administered to the subject, that is whether the compound or composition is administered prior to, concurrent with, or subsequent to a cycle of the primary chemotherapy or radiation treatment.

Also provided herein is a method for inhibiting adhesion of a tumor cell that expresses a ligand of E-selectin to an endothelial cell expressing E-selectin on its cell surface, which method comprises contacting the endothelial cell with at least one compound of the present disclosure or pharmaceutical compositions comprising at least one such compound, thereby permitting the compound to interact with E-selectin on the endothelial cell surface and inhibiting binding of the tumor cell to the endothelial cell. Without wishing to be bound by theory, inhibiting adhesion of tumor cells to endothelial cells may reduce in a significant manner, the capability of the tumor cells to extravasate into other organs, blood vessels, lymph, or bone marrow and thereby reduce, decrease, or inhibit, or slow the progression of the cancer, including reducing, decreasing, inhibiting, or slowing metastasis.

In some embodiments, a method for inhibiting activation of hepatic and/or pancreatic stellate cells is disclosed, the method comprising administering at least one compound of Formula (I) and/or a pharmaceutical composition comprising at least one compound of Formula (I).

In some embodiments, a method for inhibiting adhesion of metastasized tumor cells is disclosed, the method comprising administering at least one compound of Formula (I) and/or a pharmaceutical composition comprising at least one compound of Formula (I).

In some embodiments, a method for inhibiting cell-cell interactions and/or interactions between cells and the extracellular matrix where the cell-cell interactions and cell-matrix are induced by galectin-3 molecules bound carbohydrates found on the surface of cells is disclosed, the method comprising administering at least one compound of Formula (I) and/or a pharmaceutical composition comprising at least one compound of Formula (I). In some embodiments, the cells are tumor cells and cell-cell interactions and/or cell-matrix are responsible for the development of at least one tumor disease.

In some embodiments, a method for reducing the rate of growth of tumor cells which express galectin-3 is disclosed, the method comprising administering at least one compound of Formula (I) and/or a pharmaceutical composition comprising at least one compound of Formula (I). In some embodiments, the level of at least one GUS cyclin in the tumor cell is reduced.

As described herein, at least one of the compounds of the present disclosure or pharmaceutical compositions comprising at least one such compound may be administered in combination with at least one additional anti-cancer agent. Chemotherapy may comprise one or more chemotherapeutic agents. For example, chemotherapy agents, radiotherapeutic agents, inhibitors of phosphoinositide-3 kinase (PI3K), and inhibitors of VEGF may be used in combination with a compound of Formula (I) described herein. Non-limiting examples of inhibitors of PI3K include the compound named by Exelixis as “XL499.” Non-limiting examples of VEGF inhibitors include the compound called “cabo” (previously known as XL184). Many other chemotherapeutics are small organic molecules. As understood by a person of ordinary skill in the art, chemotherapy may also refer to a combination of two or more chemotherapeutic molecules that are administered coordinately and which may be referred to as combination chemotherapy. Numerous chemotherapeutic drugs are used in the oncology art and include, for example, alkylating agents; antimetabolites; anthracyclines, plant alkaloids; and topoisomerase inhibitors.

The compounds of the present disclosure or pharmaceutical compositions comprising at least one such compound may function independently from the anti-cancer agent or may function in coordination with the anti-cancer agent, e.g., by enhancing effectiveness of the anti-cancer agent or vice versa. Accordingly, provided herein are methods for enhancing (i.e., enhancing, promoting, improving the likelihood of, enhancing in a statistically or biologically significant manner) and/or maintaining survival of hematopoietic stem cells (HSC) in a subject who is treated with and/or will be treated with a chemotherapeutic drug(s) and/or radioactive therapy, respectively, comprising administering at least one compound of Formula (I) as described herein. In some embodiments, the subject receives and/or will receive both chemotherapy and radiation therapy. Also, provided herein is a method for reducing (i.e., reducing, inhibiting, diminishing in a statistically or biologically significant manner) chemosensitivity and/or radiosensitivity of hematopoietic stem cells (HSC) to the chemotherapeutic drug(s) and/or radioactive therapy, respectively, in a subject. Because repeated cycles of chemotherapy and radiotherapy often diminish the ability of HSCs to recover and replenish bone marrow, the glycomimetic compounds described herein may be useful for subjects who will receive more than one cycle, such as at least two, three, four or more cycles, of chemotherapy and/or radiotherapy. HSCs reside in the bone marrow and generate the cells that are needed to replenish the immune system and the blood. Anatomically, bone marrow comprises a vascular niche that is adjacent to bone endothelial sinuses (see, e.g., Kiel et al., Cell 121:1109-21 (2005); Sugiyama et al., Immunity 25:977-88 (2006); Mendez-Ferrer et al., Nature 466:829-34 (2010); Butler et al., Cell Stem Cell 6:251-64 (2010)). A recent study describes that E-selectin promotes HSC proliferation and is an important component of the vascular niche (see, e.g., Winkler et al., Nature Medicine published online 21 Oct. 2012; doi:10.1038/nm.2969). Deletion or inhibition of E-selectin enhanced HSC survival in mice that were treated with chemotherapeutic agents or radiotherapy and accelerated blood neutrophil recovery (see, e.g., Winkler et al., supra). Additionally, galectin-3 has recently been reported to interfere with hematopoiesis and promote terminal differentiation of myeloid progenitors (see, e.g., Brand et al., Cell Tissue Res 346:427-37 (2011)).

In addition, the administration of at least one compound of the present disclosure or pharmaceutical composition comprising at least one such compounds may be in conjunction with one or more other therapies, e.g., for reducing toxicities of therapy. For example, at least one palliative agent to counteract (at least in part) a side effect of a therapy (e.g., anti-cancer therapy) may be administered. Agents (chemical or biological) that promote recovery, or counteract side effects of administration of antibiotics or corticosteroids, are examples of such palliative agents. At least one compound described herein may be administered before, after, or concurrently with administration of at least one additional anti-cancer agent or at least one palliative agent to reduce a side effect of therapy. When administration is concurrent, the combination may be administered from a single container or two (or more) separate containers.

Cancer cells (also called herein tumor cells) that may be prevented (i.e., inhibited, slowed) from metastasizing, from adhering to an endothelial cell, or from infiltrating bone marrow include cells of solid tumors and liquid tumors (including hematological malignancies). Examples of solid tumors are described herein and include colorectal cancer, liver cancer, gastric cancer, lung cancer, brain cancer, kidney cancer, bladder cancer, thyroid cancer, prostate cancer, ovarian cancer, cervical cancer, uterine cancer, endometrial cancer, melanoma, breast cancer, and pancreatic cancer. Liquid tumors occur in the blood, bone marrow, and lymph nodes and include leukemia (e.g., AML, ALL, CLL, and CML), lymphoma (e.g., Hodgkin lymphoma and non-Hodgkin lymphoma), and myeloma (e.g., multiple myeloma). As used herein, the term cancer cells include mature, progenitor, and cancer stem cells.

Bones are a common location for cancer to infiltrate once leaving the primary tumor location. Once cancer resides in bone, it is frequently a cause of pain to the individual. In addition, if the particular bone affected is a source for production of blood cells in the bone marrow, the individual may develop a variety of blood cell related disorders. Breast and prostate cancer are examples of solid tumors that migrate to bones. Acute myelogenous leukemia (AML) and multiple myeloma (MM) are examples of liquid tumors that migrate to bones. Cancer cells that migrate to bone will typically migrate to the endosteal region of the bone marrow. Once cancer cells have infiltrated into the marrow, the cells become quiescent and are protected from chemotherapy. The compounds of the present disclosure may block infiltration of disseminated cancer cells into bone marrow. A variety of subjects may benefit from treatment with the compounds. Examples of such subjects include individuals with a cancer type having a propensity to migrate to bone where the tumor is still localized or the tumor is disseminated but not yet infiltrated bone, or where individuals with such a cancer type are in remission.

The cancer patient population most likely to respond to treatment using heterobifunctional antagonists of E-selectin and galectin-3 (e.g., compounds of Formula (I)) described herein can be identified based on the mechanisms of action of E-selectin and galectin-3. For example, patients may be selected that express a highly active E-selectin as determined by the genetic polymorphism for E-selectin of S128R (Alessandro et al., Int. J. Cancer 121:528-535, 2007). In addition, patients for treatment by the compounds described herein may also selected based on elevated expression of the E-selectin binding ligands (sialyl Le^{a} and sialyl Le^{x}) as determined by antibodies directed against cancer-associated antigens CA-19-9 (Zheng et al., World J. Gastroenterol. 7:431-434, 2001) and CD65. In addition, antibodies HECA-452 and FH-6 which recognize similar carbohydrate ligands of E-selectin may also be used in a diagnostic assay to select the cancer patient population most likely to respond to this treatment. Likewise, patients may be identified for treatment based on levels of galectin-3 detected in serum or plasma by a diagnostic assay such as the Abbott Laboratories ARCHITECT Galectin-3 assay, which can be used for determining galectin-3 in serum or plasma to stratify heart failure patients for proper treatment.

The compounds of the present disclosure and pharmaceutical compositions comprising at least one such compound may be useful in methods for mobilizing cells from the bone marrow to the peripheral vasculature and tissues. As discussed herein, in some embodiments, the compounds and compositions are useful for mobilizing hematopoietic cells, including hematopoietic stem cells and hematopoietic progenitor cells. In some embodiments, the compounds act as mobilizing agents of normal blood cell types. In some embodiments, the agents are used in methods for mobilizing mature white blood cells (which may also be called leukocytes herein), such as granulocytes (e.g., neutrophils, eosinophils, basophils), lymphocytes, and monocytes from the bone marrow or other immune cell compartments such as the spleen and liver. Methods are also provided for using the compounds of the present disclosure and pharmaceutical compositions comprising at least one such compound in methods for mobilizing tumor cells from the bone marrow. The tumor cells may be malignant cells (e.g., tumor cells that are metastatic cancer cells, or highly invasive tumor cells) in cancers. These tumor cells may be of hematopoietic origin or may be malignant cells of another origin residing in the bone.

In some embodiments, the methods using the compounds described herein are useful for mobilizing hematopoietic cells, such as hematopoietic stem cells and progenitor cells and leukocytes (including granulocytes such as neutrophils), which are collected (i.e., harvested, obtained) from the subject receiving a compound of Formula (I) and at a later time are administered back into the same subject (autologous donor) or administered to a different subject (allogeneic donor). Hematopoietic stem cell replacement and hematopoietic stem cell transplantation have been successfully used for treating a number of diseases (including cancers) as described herein and in the art. By way of example, stem cell replacement therapy or transplantation follows myeloablation of a subject, such as occurs with administration of high dose chemotherapy and/or radiotherapy. Desirably, an allogeneic donor shares sufficient HLA antigens with the recipient/subject to minimize the risk of host versus graft disease in the recipient (i.e., the subject receiving the hematopoietic stem cell transplant). Obtaining the hematopoietic cells from the donor subject (autologous or allogeneic) is performed by apheresis or leukapheresis. HLA typing of a potential donor and the recipient and apheresis or leukapheresis are methods routinely practiced in the clinical art.

By way of non-limiting example, autologous or allogenic hematopoietic stem cells and progenitors cells may be used for treating a recipient subject who has certain cancers, such as Hodgkin lymphoma, non-Hodgkin lymphoma, or multiple myeloma. Allogeneic hematopoietic stem cells and progenitors cells may be used, for example, for treating a recipient subject who has acute leukemia (e.g., AML, ALL); chronic lymphocytic leukemia (CLL); amegakaryocytosis/congenital thrombocytopenia; aplastic anemia/refractory anemia; familial erythrophagocytic lymphohistiocytosis; myelodysplastic syndrome/other myelodysplastic disorders; osteopetrosis; paroxysmal nocturnal hemoglobinuria; and Wiskott-Aldrich syndrome, for example. Exemplary uses for autologous hematopoietic stem cells and progenitors cells include treating a recipient subject who has amyloidosis; germ cell tumors (e.g., testicular cancer); or a solid tumor. Allogeneic hematopoietic stem cell transplants have also been investigated for use in treating solid tumors (see, e.g., Ueno et al., Blood 102:3829-36 (2003)).

In some embodiments of the methods described herein, the subject is not a donor of peripheral hematopoietic cells but has a disease, disorder, or condition for which mobilization of hematopoietic cells in the subject will provide clinical benefit. Stated another way, while this clinical situation is similar to autologous hematopoietic cell replacement, the mobilized hematopoietic cells are not removed and given back to the same subject at a later time as occurs, for example, with a subject who receives myeloablation therapy. Accordingly, methods are provided for mobilizing hematopoietic cells, such as hematopoietic stem cells and progenitor cells and leukocytes (including granulocytes, such as neutrophils), by administering at least once compound of Formula (I). Mobilizing hematopoietic stem cells and progenitor cells may be useful for treating an inflammatory condition or for tissue repair or wound healing. See, e.g., Mimeault et al., Clin. Pharmacol. Therapeutics 82:252-64 (2007).

In some embodiments, the methods described herein are useful for mobilizing hematopoietic leukocytes (white blood cells) in a subject, which methods may be used in treating diseases, disorders, and conditions for which an increase in white blood cells, such as neutrophils, eosinophils, lymphocytes, monocytes, basophils, will provide clinical benefit. For example, for cancer patients, the compounds of Formula (I) are beneficial for stimulating neutrophil production to compensate for hematopoietic deficits resulting from chemotherapy or radiation therapy. Other diseases, disorders, and conditions to be treated include infectious diseases and related conditions, such as sepsis. When the subject to whom at least one compound of Formula (I) is administered is a donor, neutrophils may be collected for administration to a recipient subject who has reduced hematopoietic function, reduced immune function, reduced neutrophil count, reduced neutrophil mobilization, severe chronic neutropenia, leucopenia, thrombocytopenia, anemia, and acquired immune deficiency syndrome. Mobilization of mature white blood cells may be useful in subjects to improve or to enhance tissue repair, and to minimize or prevent vascular injury and tissue damage, for example following liver transplantation, myocardial infarction or limb ischemia. See, e.g., Pelus, Curr. Opin. Hematol. 15:285-92 (2008); Lemoli et al., Haematologica 93:321-24 (2008).

The compounds of Formula (I) may be used in combination with one or more other agents that mobilize hematopoietic cells. Such agents include, for example, G-CSF; AMD3100 or other CXCR4 antagonists; GRO-β (CXCL2) and an N-terminal 4-amino truncated form (SB-251353); IL-8SDF-1α peptide analogs, CTCE-0021 and CTCE-0214; and the SDF1 analog, Met-SDF-1β (see, e.g., Pelus, supra and references cited therein). In some embodiments, a compound of Formula (I) may be administered with other mobilizing agents used in the art, which may permit administration of a lower dose of GCSF or AMD3100, for example, than required in the absence of a compound of Formula (I). The appropriate therapeutic regimen for administering a compound of Formula (I) in combination with another mobilizing agent or agents can be readily determined by a person skilled in the clinical art.

The compounds of the present disclosure and pharmaceutical compositions comprising at least one such compound may be useful in methods for preventing and/or treating mucositis. In some embodiments, at least one compound of Formula (I) and/or a pharmaceutical composition comprising at least one compound of Formula (I) may be used in methods described herein for decreasing the likelihood of occurrence of mucositis in a subject who is in need thereof by administering the compound or composition to the subject. In some embodiments, the mucositis is chosen from oral mucositis, esophageal mucositis, and gastrointestinal mucositis. In some embodiments, the mucositis is alimentary mucositis.

It is believed that approximately half of all cancer patients undergoing therapy suffer some degree of mucositis. Mucositis is believed to occur, for example, in virtually all patients treated with radiation therapy for head and neck tumors, all patients receiving radiation along the GI tract, and approximately 40% of those subjected to radiation therapy and/or chemotherapy for tumors in other locations (e.g., leukemias or lymphomas). It is also is believed to be highly prevalent in patients treated with high dose chemotherapy and/or irradiation for the purpose of myeloablation, such as in preparation for stem cell or bone marrow transplantation. The compounds of the present disclosure and pharmaceutical compositions comprising at least one such compound may be useful in methods for treating and/or preventing mucositis in a subject afflicted with cancer. In some embodiments, the subject is afflicted with a cancer chosen from head and neck cancer, breast cancer, lung cancer, ovarian cancer, prostate cancer, lymphatic cancer, leukemic cancer, and/or gastrointestinal cancer. In some embodiments, the mucositis is associated with radiation therapy and/or chemotherapy. In some embodiments, the chemotherapy comprises administering a therapeutically effective amount of at least one compound chosen from platinum, cisplatin, carboplatin, oxaliplatin, mechlorethamine, cyclophosphamide, chlorambucil, azathioprine, mercaptopurine, vincristine, vinblastine, vinorelbine, vindesine, etoposide, teniposide, paclitaxel, docetaxel, irinotecan, topotecan, amsacrine, etoposide, etoposide phosphate, teniposide, 5-fluorouracil (5-FU), leucovorin, methotrexate, gemcitabine, taxane, leucovorin, mitomycin C, tegafur-uracil, idarubicin, fludarabine, mitoxantrone, ifosfamide and doxorubicin.

In some embodiments, the method further comprises administering a therapeutically effective amount of at least one MMP inhibitor, inflammatory cytokine inhibitor, mast cell inhibitor, NSAID, NO inhibitor, or antimicrobial compound.

In some embodiments, the method further comprises administering a therapeutically effective amount of velafermin and/or palifermin.

In some embodiments, the method further comprises administering a therapeutically effective amount of Davanat®, mannose, and/or galactose.

The compounds of the present disclosure and pharmaceutical compositions comprising at least one such compound may be useful in methods for treating and/or preventing thrombosis. As described herein methods are provided for inhibiting formation of a thrombus or inhibiting the rate at which a thrombus is formed. These methods may therefore be used for preventing thrombosis (i.e., reducing or decreasing the likelihood of occurrence of a thrombus in a statistically or clinically significant manner).

Thrombus formation may occur in infants, children, teenagers and adults. An individual may have a hereditary predisposition to thrombosis. Thrombosis may be initiated, for example, due to a medical condition (such as cancer or pregnancy), a medical procedure (such as surgery) or an environmental condition (such as prolonged immobility). Other individuals at risk for thrombus formation include those who have previously presented with a thrombus.

The compounds of the present disclosure and pharmaceutical compositions comprising at least one such compound may be useful in methods for treating individuals undergoing thrombosis or who are at risk of a thrombotic event occurring. Such individuals may or may not have a risk of bleeding. In some embodiments, the individual has a risk of bleeding. In some embodiments, the thrombosis is a venous thromboembolism (VTE). VTE causes deep vein thrombosis and pulmonary embolism. Low molecular weight (LMW) heparin is the current mainstay therapy for the prevention and treatment of VTE. In many circumstances, however, the use of LMW heparin is contraindicated. LMW heparin is a known anti-coagulant and delays clotting over four times longer than control bleeding times. Patients undergoing surgery, patients with thrombocytopenia, patients with a history of stroke, and many cancer patients should avoid administration of heparin due to the risk of bleeding. By contrast, administration of the compounds of Formula (I) significantly reduces the time to clotting than occurs when LMW heparin is administered, and thus provide a significant improvement in reducing bleeding time compared with LMW heparin. Accordingly, the compounds and pharmaceutical compositions described herein may not only be useful for treating a patient for whom the risk of bleeding is not significant, but also may be useful in when the risk of bleeding is significant and the use of anti-thrombosis agents with anti-coagulant properties (such as LMW heparin) is contraindicated.

The compounds of the present disclosure and pharmaceutical compositions comprising at least one such compound may be administered in combination with at least one additional anti-thrombosis agent. The compounds of the present disclosure and pharmaceutical compositions comprising at least one such compound may function independently from the anti-thrombosis agent or may function in coordination with the at least one anti-thrombosis agent. In addition, the administration of one or more of the compounds or compositions may be in conjunction with one or more other therapies, e.g., for reducing toxicities of therapy. For example, at least one palliative agent to counteract (at least in part) a side effect of therapy may be administered. Agents (chemical or biological) that promote recovery and/or counteract side effects of administration of antibiotics or corticosteroids are examples of such palliative agents. The compounds of the present disclosure and pharmaceutical composition comprising at least one such compound may be administered before, after, or concurrently with administration of at least one additional anti-thrombosis agent or at least one palliative agent to reduce a side effect of therapy. Where administration is concurrent, the combination may be administered from a single container or two (or more) separate containers.

The compounds of the present disclosure and pharmaceutical compositions comprising at least one such compound may be useful for treating and/or preventing at least one cardiovascular disease, disorder and/or condition. Non-limiting examples of cardiovascular disease include atherosclerosis, myocardial infarction, myocardial ischemia, coronary artery stenosis (occlusion of the coronary arteries), chronic cardiovascular and/or arterial inflammation, acute cardiovascular and/or arterial inflammation, hypercholesterolemia, restenosis (narrowing of the vessel lumen), arrhythmia, thrombosis, hyperlipidemia, hypertension, dyslipoproteinemia, angina (cardiac chest pain), and vascular complications due to a cardiovascular disease (e.g., myocardial infarction or myocardial ischemia).

In some embodiments, at least one compound of Formula (I) and/or a pharmaceutical composition comprising at least one compound of Formula (I) may be administered prior to or subsequent to an acute cardiovascular event in the subject. In some embodiments, at least one compound of Formula (I) and/or a pharmaceutical composition comprising at least one compound of Formula (I) may be administered prior to or subsequent to the development or diagnosis of a cardiovascular disease, disorder and/or condition in the subject. In some embodiments, the acute cardiovascular event is a myocardial infarction.

In some embodiments, a method for treatment and/or prevention of atherosclerosis is disclosed, the method comprising administering to a subject in need thereof an effective amount of at least one compound of Formula (I) and/or a pharmaceutical composition comprising at least one compound of Formula (I). Atherosclerosis generally describes a disease of the arterial blood vessels. As used herein, “atherosclerosis” includes, but is not limited to, chronic and/or acute atherosclerotic inflammation prior to or subsequent to the formation of at least one atherosclerotic plaque in the subject. Atherosclerosis also includes, but is not limited to, chronic progressive atherosclerosis and/or atherosclerotic inflammation. Atherosclerosis also includes, but is not limited to, acute atherosclerosis and/or atherosclerotic inflammation subsequent to an acute vascular event in the subject (such as, for example, myocardial infarction).

In some embodiments, at least one compound of Formula (I) and/or a pharmaceutical composition comprising at least one compound of Formula (I) may be administered prior to or subsequent to the formation of at least one atherosclerotic plaque, lesion or atheroma in the subject.

In some embodiments, the formation, progression, destabilization and/or rupture of at least one atherosclerotic plaque within the subject is reduced.

Atherosclerotic plaques may be characterized as stable or unstable (i.e., vulnerable to destabilization). Unstable atherosclerotic plaques may be susceptible to disruption or rupture, which exposes thrombogenic material (i.e., thrombi) (e.g., collagen) to the circulation. This can produce interruptions in blood flood (ischemia) in local or distal arteries, which can result in cardiovascular complications, such as, for example, myocardial infarction (MI).

Destabilization of atherosclerotic plaques may occur via many mechanisms. Non-limiting examples of such mechanisms include superficial erosion of the endothelial cells that form the monolayer covering the intima, disruption of the microvessels that form in the atherosclerotic plaque, rupture (i.e., fracture) of the atherosclerotic plaque's fibrous cap, thinning or weakening of the fibrous cap (thus making it susceptible to rupture), and the presence or increase in inflammatory factors that mediate destabilization. (Libby P., Nature, 420: 868-874 (2002)).

A non-limiting example of inflammatory factors that mediate destabilization is the presence of inflammatory cells. The progression of atherosclerosis may be associated with systemically increased inflammatory myeloid cells that are recruited to atherosclerotic plaques. (Murphy, A. J. et al., J. Clin. Invest., 121: 4138-4149 (2011); Averill, L. E. et al., Am. J. Pathol., 135: 369-377 (1989); Feldman, D. L. et al., Arterioscler. Thromb., 11: 985-994 (1991); Swirski, F. K. et al., J. Clin. Invest., 117: 195-205 (2007)). The presence of inflammatory myeloid cells may be detrimental to a stable plaque. (Llodra, J. et al., Proc. Natl. Acad. Sci. U.S.A., 101: 11779-11784 (2004)).

In some embodiments, the stability of at least one atherosclerotic plaque within the subject is increased. Non-limiting examples of stable features of atherosclerotic plaques (i.e., stable phenotype) include smaller plaque size, reduced (i.e., decreased, diminished, smaller) necrotic core size (measured by, for example, necrotic core area), and a thicker fibrous cap of the atherosclerotic plaque. (See, e.g., Moore K. J. et al., Cell, 145: 341-355 (2011)).

In some embodiments, the size of at least one atherosclerotic plaque within the subject is decreased. In some embodiments, the necrotic core size of at least one atherosclerotic plaque within the subject is decreased. In some embodiments, the fibrous cap thickness of at least one atherosclerotic plaque within the subject is increased.

In some embodiments, the administration of an effective amount of at least one compound of Formula (I) and/or a pharmaceutical composition comprising at least one compound of Formula (I) reduces the levels of extramedullary proliferation of hematopoietic stem and/or progenitor cells within the subject. In some embodiments, extramedullary proliferation of hematopoietic stem and/or progenitor cells is reduced in the spleen and/or the liver. Non-limiting examples of extramedullary proliferation of hematopoietic stem and/or progenitor cells include extramedullary hematopoiesis and extramedullary myelopoiesis.

In some embodiments, the administration of an effective amount of at least one compound of Formula (I) and/or a pharmaceutical composition comprising at least one compound of Formula (I) reduces the recruitment and/or infiltration of myeloid cells to at least one atherosclerotic plaque within the subject. Non-limiting examples of myeloid cells include monocytes, macrophages, neutrophils, basophils, eosinophils, erythrocytes, dendritic cells, and megakaryocytes and platelets.

In some embodiments, the at least one compound of Formula (I) and/or a pharmaceutical composition comprising at least one compound of Formula (I) is administered after angioplasty, stenting procedure, atherectomy, bypass surgery, or other vessel-corrective techniques.

In some embodiments, the at least one compound of Formula (I) and/or a pharmaceutical composition comprising at least one compound of Formula (I) is administered before angioplasty, stenting procedure, atherectomy, bypass surgery, or other vessel-corrective techniques.

In some embodiments, a method for treatment and prevention of myocardial infarction is disclosed, the method comprising administering to a subject in need thereof an effective amount of at least one compound of Formula (I) and/or a pharmaceutical composition comprising at least one compound of Formula (I). In some embodiments, the subject has previously suffered a myocardial infarction. In some embodiments, a compound of Formula (I) may be administered before the occurrence of a myocardial infarction in the subject. In some embodiments, a compound of Formula (I) may be administered after the occurrence of a first or subsequent myocardial infarction in the subject.

In some embodiments, at least one compound of Formula (I) and/or a pharmaceutical composition comprising at least one compound of Formula (I) is administered to the subject: within one (1) day of the subject suffering a myocardial infarction, within one (1) week of the subject suffering a myocardial infarction, within two (2) weeks of the subject suffering a myocardial infarction, within three (3) weeks of the subject suffering a myocardial infarction, within four (4) weeks of the subject suffering a myocardial infarction, within eight (8) weeks of the subject suffering a myocardial infarction, or within twelve (12) weeks of the subject suffering a myocardial infarction.

In some embodiments, a method for the treatment of sickle cell disease or complications associated therewith is disclosed, the method comprising administering to a subject in need thereof an effective amount of at least one compound of Formula (I) and/or a pharmaceutical composition comprising at least one compound of Formula (I).

In some embodiments, a method for treatment and prevention of vaso-occlusive crisis or complications associated therewith is disclosed, the method comprising administering to a subject in need thereof an effective amount of at least one compound of Formula (I) and/or a pharmaceutical composition comprising at least one compound of Formula (I).

In some embodiments, a method for treatment and/or prevention of pathological angiogenesis is disclosed, the method comprising administering to a subject in need thereof an effective amount of at least one compound of Formula (I) and/or a pharmaceutical composition comprising at least one compound of Formula (I).

In some embodiments, the pathological angiogenesis in the eye. Examples of ocular diseases, disorders, or conditions associated with pathological angiogenesis include age-related macular degeneration, ocular histoplasmosis syndrome, neovascular glaucoma, retrolental fibroplasia, pathologic myopia, angioid streaks, idiopathic disorders, choroiditis, choroidal rupture, overlying choroid nevi, grafi rejection, herpes simplex keratitis, leishmaniasis, onchocerciasis, certain inflammatory diseases such as dry eye syndrome, and trauma to the eye (e.g., cornea).

In some embodiments, the present disclosure is directed to methods for treatment and prevention of pathological angiogenesis in patients with cancer.

In some embodiments, a method for treatment and/or prevention of an epileptic syndrome is disclosed, the method comprising administering to a subject in need thereof at least one compound of Formula (I) and/or a pharmaceutical composition comprising at least one compound of Formula (I). Examples of an epileptic syndrome include epilepsy, Rasmussen's syndrome and West syndrome. Other syndromes which are multi-system disorders but with the primary disability resulting from neurological effects including epilepsy, are considered epileptic syndromes for purposes of the present invention. An example of such a syndrome is tuberous sclerosis syndrome.

The compounds of the present disclosure and pharmaceutical compositions comprising at least one such compound may be administered in combination with at least one additional antiepileptic agent (e.g. acetazolamide, carbamazepine, clobazam, clonazepam, eslicarbazepine acetate, ethosuximide, gabapentin, lacosamide, lamotrigine, levetiracetam, nitrazepam, oxcarbazepine, perampanel, piracetam, phenobarbital, phenytoin, pregabalin, primidone, rufinamide, sodium valproate, stiripentol, tiagabine, topiramate, vigabatrin, zonisamide). The compounds of the present disclosure and pharmaceutical compositions comprising at least one such compound may function independently from the antiepileptic agent or may function in coordination with the at least one antiepileptic agent. In addition, the administration of one or more of the compounds or compositions may be in conjunction with one or more other therapies, e.g., for reducing toxicities of therapy. For example, at least one palliative agent to counteract (at least in part) a side effect of therapy may be administered. Agents (chemical or biological) that promote recovery or enhancement of appetite, or counteract nausea or fatigue, are examples of such agents. The compounds of the present disclosure and pharmaceutical composition comprising at least one such compound may be administered before, after, or concurrently with administration of at least one additional anti-thrombosis agent or at least one palliative agent to reduce a side effect of therapy. Where administration is concurrent, the combination may be administered from a single container or two (or more) separate containers.

In some embodiments, a method for treatment and/or prevention of a neurodegenerative disease is disclosed, the method comprising administering to a subject in need thereof an effective amount of at least one compound of Formula (I) and/or a pharmaceutical composition comprising at least one compound of Formula (I). Examples of neurodegenerative diseases include such as selected from Parkinson's disease, dementia with Lewy bodies, pure autonomic failure (PAF), Alzheimer's disease, neurodegeneration with brain iron accumulation, type I (also referred to as adult neuroaxonal dystrophy or Hallervorden-Spatz syndrome), traumatic brain injury, amyotrophic lateral sclerosis, Pick disease, multiple system atrophy (including Shy-Drager syndrome, striatonigral degeneration, and olivopontocerebellar atrophy) and stroke, multiple sclerosis, epilepsy and infantile neuroaxonal dystrophy.

In some embodiments, a method for treatment and/or prevention of α-synucleinopathies is disclosed, the method comprising administering to a subject in need thereof an effective amount of at least one compound of Formula (I) and/or a pharmaceutical composition comprising at least one compound of Formula (I). Examples of α-synucleinopathies include such as selected from Parkinson's disease, dementia with Lewy bodies, pure autonomic failure (PAF), Alzheimer's disease, neurodegeneration with brain iron accumulation, type I (also referred to as adult neuroaxonal dystrophy or Hallervorden-Spatz syndrome), traumatic brain injury, amyotrophic lateral sclerosis, Pick disease, multiple system atrophy (including Shy-Drager syndrome, striatonigral degeneration, and olivopontocerebellar atrophy) and stroke, multiple sclerosis, epilepsy and infantile neuroaxonal dystrophy.

The compounds of the present disclosure and pharmaceutical compositions comprising at least one such compound may be administered in combination with at least one additional agent for the treatment of neurodegeneration or symptoms thereof (e.g. donepezil, galantamine, memantine, rivastigmine, levodopa, carbidopa, dopamine agonists, COMT inhibitors, MAO inhibitors, anticholinergic agents, corticosteroids, beta interferons, ocrelizumab, glatiramer acetate, dimethyl fumarate, fingolimod, teriflunomide, natalizumab, alemtuzumab, mitoxantrone, riluzole, edaravone). The compounds of the present disclosure and pharmaceutical composition comprising at least one such compound may be administered before, after, or concurrently with administration of at least one additional agent for the treatment of neurodegeneration or symptoms thereof. Where administration is concurrent, the combination may be administered from a single container or two (or more) separate containers.

In some embodiments, a method for treatment and prevention of a fibrosing disease or condition is disclosed, the method comprising administering to a subject in need thereof an effective amount of at least one compound of Formula (I) and/or a pharmaceutical composition comprising at least one compound of Formula (I). Examples of fibrosing diseases and conditions include such as selected from rheumatoid arthritis, lupus, pathogenic fibrosis, fibrosing disease, heart disease, heart remodeling post MI, nonalcoholic fatty liver disease (NASH), idiopathic pulmonary fibrosis (IPF), fibrosis associated with thrombosis, fibrosis associated with macular degeneration, fibrotic lesions such as those formed after Schistosoma japonicum infection, radiation damage, autoimmune diseases, Lyme disease, chemotherapy induced fibrosis, HIV or infection-induced focal Sclerosis, failed back syndrome due to spinal Surgery scarring, abdominal adhesion post-Surgery scarring, fibrocystic formations, fibrosis after spinal injury, Surgery-induced fibrosis, mucosal fibrosis, peritoneal fibrosis caused by dialysis, Adalimumab-associated pulmonary fibrosis, and nephrogenic fibrosing dermopathy.

In some embodiments, the fibrosis is fibrosis of the liver resulting from conditions including but not limited to alcohol, drug, or chemically induced cirrhosis, ischemia-reperfusion injury after hepatic transplant, necrotizing hepatitis, hepatitis B, hepatitis C, primary biliary cirrhosis, primary sclerosing cholangitis, and nonalcoholic steatohepatitis.

In some embodiments, the fibrosis is fibrosis in the kidney resulting from conditions including but not limited to proliferative and Sclerosing glomerulonephritis, nephrogenic fibrosing dermopathy, diabetic nephropathy, renal tubulointerstitial fibrosis, and focal segmental glomerulosclerosis.

In some embodiments, the fibrosis is fibrosis of the lung resulting from conditions including but not limited to pulmonary interstitial fibrosis, sarcoidosis, pulmonary fibrosis, idiopathic pulmonary fibrosis, asthma, chronic obstructive pulmonary disease, diffuse alveolar damage disease, pulmonary hypertension, neonatal bronchopulmonary dysplasia, chronic asthma, and emphysema. There are several subnames or synonyms for pulmonary fibrosis including, but not limited to, cryptogenic fibrosing alveolitis, diffuse interstitial fibrosis, idiopathic interstitial pneumonitis, Hamman Rich syndrome, silicosis, asbestosis, berylliosis, coal worker's pneumoconiosis, coal miner's disease, miner's asthma, anthracosis, and anthracosilicosis.

In some embodiments, the fibrosis is fibrosis of the heart or pericardium resulting from conditions including but not limited to myocardial fibrosis, atherosclerosis, coronary artery restenosis, congestive cardiomyopathy, heart failure, and other post-ischemic conditions.

In some embodiments, the fibrosis is fibrosis of the eye resulting from conditions including but not limited to macular degeneration, exophthalmos of Grave's disease, proliferative vitreoretinopathy, anterior capsule cataract, corneal fibrosis, corneal scarring due to surgery, trabeculectomy-induced fibrosis, progressive sub-retinal fibrosis, multifocal granulomatous chorioretinitis, fibrosis due to wide angle glaucoma trabeculotomy, and other eye fibrosis.

In some embodiments, the fibrosis is fibrosis of the brain resulting from conditions including but not limited to glial scar tissue.

In some embodiments, the fibrosis is fibrosis of the skin resulting from conditions including but not limited to Depuytren's contracture, Scleroderma, keloid scarring, psoriasis, hyper-trophic scarring due to burns, atherosclerosis, restenosis, and pseudoscleroderma caused by spinal cord injury.

In some embodiments, the fibrosis is fibrosis of tissue including but not limited to the mouth or esophagus, pancreas, gastrointestinal tract, breast, bone, bone marrow, genitourinary system.

The terms “treat” and “treatment” include medical management of a disease, disorder, and/or condition of a subject as would be understood by a person of ordinary skill in the art (see, e.g., Stedman's Medical Dictionary). In general, an appropriate dose and treatment regimen provide at least one of the compounds of the present disclosure in an amount sufficient to provide therapeutic and/or prophylactic benefit. For both therapeutic treatment and prophylactic or preventative measures, therapeutic and/or prophylactic benefit includes, for example, an improved clinical outcome, wherein the object is to prevent or slow or lessen an undesired physiological change or disorder, or to prevent or slow or lessen the expansion or severity of such disorder. As discussed herein, beneficial or desired clinical results from treating a subject include, but are not limited to, abatement, lessening, or alleviation of symptoms that result from or are associated with the disease, condition, and/or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can include prolonging survival when compared to expected survival if a subject were not receiving treatment.

In some embodiments of the methods described herein, the subject is a human. In some embodiments of the methods described herein, the subject is a non-human animal. Non-human animals that may be treated include mammals, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

The effectiveness of the compounds of the present disclosure in treating and/or preventing diseases, disorders, and/or conditions treatable by inhibiting an activity of E-selectin and/or galectin-3 can readily be determined by a person of ordinary skill in the relevant art. Determining and adjusting an appropriate dosing regimen (e.g., adjusting the amount of compound per dose and/or number of doses and frequency of dosing) can also readily be performed by a person of ordinary skill in the relevant art. One or any combination of diagnostic methods, including physical examination, assessment and monitoring of clinical symptoms, and performance of analytical tests and methods described herein, may be used for monitoring the health status of the subject.

Also provided herein are pharmaceutical compositions comprising at least one compound of Formula (I). In some embodiments, the pharmaceutical compositions further comprise at least one additional pharmaceutically acceptable ingredient.

In pharmaceutical compositions, any one or more of the compounds of the present disclosure may be administered in the form of a pharmaceutically acceptable derivative, such as a salt, and/or it or they may also be used alone and/or in appropriate association, as well as in combination, with other pharmaceutically active compounds.

An effective amount or therapeutically effective amount refers to an amount of at least one compound of the present disclosure or a pharmaceutical composition comprising at least one such compound that, when administered to a subject, either as a single dose or as part of a series of doses, is effective to produce at least one therapeutic effect. Optimal doses may generally be determined using experimental models and/or clinical trials. Design and execution of pre-clinical and clinical studies for each of the therapeutics (including when administered for prophylactic benefit) described herein are well within the skill of a person of ordinary skill in the relevant art. The optimal dose of a therapeutic may depend upon the body mass, weight, and/or blood volume of the subject. In general, the amount of at least one compound of Formula (I) as described herein, that is present in a dose, may range from about 0.1 mg to about 100 mg per kg weight of the subject. The minimum dose that is sufficient to provide effective therapy may be used in some embodiments. Subjects may generally be monitored for therapeutic effectiveness using assays suitable for the disease, disorder and/or condition being treated or prevented, which assays will be familiar to those having ordinary skill in the art and are described herein. The level of a compound that is administered to a subject may be monitored by determining the level of the compound (or a metabolite of the compound) in a biological fluid, for example, in the blood, blood fraction (e.g., serum), and/or in the urine, and/or other biological sample from the subject. Any method practiced in the art to detect the compound, or metabolite thereof, may be used to measure the level of the compound during the course of a therapeutic regimen.

The dose of a compound described herein may depend upon the subject's condition, that is, stage of the disease, severity of symptoms caused by the disease, general health status, as well as age, gender, and weight, and other factors apparent to a person of ordinary skill in the medical art. Similarly, the dose of the therapeutic for treating a disease, disorder, and/or condition may be determined according to parameters understood by a person of ordinary skill in the medical art.

Pharmaceutical compositions may be administered in any manner appropriate to the disease, disorder, and/or condition to be treated as determined by persons of ordinary skill in the medical arts. An appropriate dose and a suitable duration and frequency of administration will be determined by such factors as discussed herein, including the condition of the patient, the type and severity of the patient's disease, the particular form of the active ingredient, and the method of administration. In general, an appropriate dose (or effective dose) and treatment regimen provides the composition(s) as described herein in an amount sufficient to provide therapeutic and/or prophylactic benefit (for example, an improved clinical outcome, such as more frequent complete or partial remissions, or longer disease-free and/or overall survival, or a lessening of symptom severity or other benefit as described in detail above).

The pharmaceutical compositions described herein may be administered to a subject in need thereof by any one of several routes that effectively delivers an effective amount of the compound. Non-limiting examples of suitable administrative routes include topical, oral, nasal, intrathecal, enteral, buccal, sublingual, transdermal, rectal, vaginal, intraocular, subconjunctival, sublingual, and parenteral administration, including subcutaneous, intravenous, intramuscular, intrasternal, intracavernous, intrameatal, and intraurethral injection and/or infusion.

The pharmaceutical compositions described herein may, for example, be sterile aqueous or sterile non-aqueous solutions, suspensions, or emulsions, and may additionally comprise at least one pharmaceutically acceptable excipient (i.e., a non-toxic material that does not interfere with the activity of the active ingredient). Such compositions may, for example, be in the form of a solid, liquid, or gas (aerosol). Alternatively, the compositions described herein may, for example, be formulated as a lyophilizate, or compounds described herein may be encapsulated within liposomes using technology known in the art. The pharmaceutical compositions may further comprise at least one additional pharmaceutically acceptable ingredient, which may be biologically active or inactive. Non-limiting examples of such ingredients include buffers (e.g., neutral buffered saline or phosphate buffered saline), carbohydrates (e.g., glucose, mannose, sucrose or dextrans), mannitol, proteins, polypeptides, amino acids (e.g., glycine), antioxidants, chelating agents (e.g., EDTA and glutathione), stabilizers, dyes, flavoring agents, suspending agents, and preservatives.

Any suitable excipient or carrier known to those of ordinary skill in the art for use in compositions may be employed in the compositions described herein. Excipients for therapeutic use are well known, and are described, for example, in Remington: The Science and Practice of Pharmacy (Gennaro, 21^{st} Ed. Mack Pub. Co., Easton, PA (2005)). In general, the type of excipient may be selected based on the mode of administration, as well as the chemical composition of the active ingredient(s). Compositions may be formulated for the particular mode of administration. For parenteral administration, pharmaceutical compositions may further comprise water, saline, alcohols, fats, waxes, and buffers. For oral administration, pharmaceutical compositions may further comprise at least one component chosen, for example, from any of the aforementioned ingredients, excipients and carriers, such as mannitol, lactose, starch, magnesium stearate, sodium saccharine, talcum, cellulose, kaolin, glycerin, starch dextrins, sodium alginate, carboxymethylcellulose, ethyl cellulose, glucose, sucrose, and magnesium carbonate.

The pharmaceutical compositions (e.g., for oral administration or delivery by injection) may be in the form of a liquid. A liquid composition may include, for example, at least one the following: a sterile diluent such as water for injection, saline solution, including for example physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral preparation can be enclosed in ampoules, disposable syringes or multiple dose vials made of glass or plastic. In some embodiments, the pharmaceutical composition comprises physiological saline. In some embodiments, the pharmaceutical composition is an injectable composition, and in some embodiments, the injectable composition is sterile.

For oral formulations, at least one of the compounds of the present disclosure can be used alone or in combination with at least one additive appropriate to make tablets, powders, granules and/or capsules, for example, those chosen from conventional additives, disintegrators, lubricants, diluents, buffering agents, moistening agents, preservatives, coloring agents, and flavoring agents. The pharmaceutical compositions may be formulated to include at least one buffering agent, which may provide for protection of the active ingredient from low pH of the gastric environment and/or an enteric coating. A pharmaceutical composition may be formulated for oral delivery with at least one flavoring agent, e.g., in a liquid, solid or semi-solid formulation and/or with an enteric coating.

Oral formulations may be provided as gelatin capsules, which may contain the active compound or biological along with powdered carriers. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract.

A pharmaceutical composition may be formulated for sustained or slow release. Such compositions may generally be prepared using well known technology and administered by, for example, oral, rectal or subcutaneous implantation, or by implantation at the desired target site. Sustained-release formulations may contain the active therapeutic dispersed in a carrier matrix and/or contained within a reservoir surrounded by a rate controlling membrane. Excipients for use within such formulations are biocompatible, and may also be biodegradable; the formulation may provide a relatively constant level of active component release. The amount of active therapeutic contained within a sustained release formulation depends upon the site of implantation, the rate and expected duration of release, and the nature of the condition to be treated or prevented.

The pharmaceutical compositions described herein can be formulated as suppositories by mixing with a variety of bases such as emulsifying bases or water-soluble bases. The pharmaceutical compositions may be prepared as aerosol formulations to be administered via inhalation. The pharmaceutical compositions may be formulated into pressurized acceptable propellants such as dichlorodifluoromethane, propane, nitrogen and the like.

The compounds of the present disclosure and pharmaceutical compositions comprising these compounds may be administered topically (e.g., by transdermal administration). Topical formulations may be in the form of a transdermal patch, ointment, paste, lotion, cream, gel, and the like. Topical formulations may include one or more of a penetrating agent or enhancer (also call permeation enhancer), thickener, diluent, emulsifier, dispersing aid, or binder. Physical penetration enhancers include, for example, electrophoretic techniques such as iontophoresis, use of ultrasound (or “phonophoresis”), and the like. Chemical penetration enhancers are agents administered either prior to, with, or immediately following administration of the therapeutic, which increase the permeability of the skin, particularly the stratum corneum, to provide for enhanced penetration of the drug through the skin. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneräs et al., J. Pharm. Pharmacol. 54:499-508 (2002); Karande et al., Pharm. Res. 19:655-60 (2002); Vaddi et al., It. J. Pharm. 91:1639-51 (2002); Ventura et al., J. Drug Target 9:379-93 (2001); Shokri et al., Int. J. Pharm. 228(1-2):99-107 (2001); Suzuki et al., Biol. Pharm. Bull. 24:698-700 (2001); Alberti et al., J. Control Release 71:319-27 (2001); Goldstein et al., Urology 57:301-5 (2001); Kiijavainen et al., Eur. J. Pharm. Sci. 10:97-102 (2000); and Tenjarla et al., Int. J. Pharm. 192:147-58 (1999).

Kits comprising unit doses of at least one compound of the present disclosure, for example in oral or injectable doses, are provided. Such kits may include a container comprising the unit dose, an informational package insert describing the use and attendant benefits of the therapeutic in treating the pathological condition of interest, and/or optionally an appliance or device for delivery of the at least one compound of Formula (I) and/or pharmaceutical composition comprising the same.

### EXAMPLES

### Example 1

### Synthesis of Building Block 14

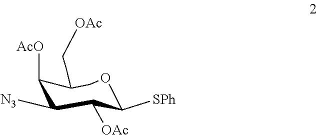

Compound 2: Compound 1 (1.5 g, 4.02 mmoles) was dissolved in DCM (30 mL). Thiophenol (0.9 g, 0.82 mL, 8.04 mmoles) was added followed by dropwise addition of boron trifluoride diethyl etherate (1.79 g, 1.49 mL, 12.06 mmoles). The reaction mixture was stirred at room temperature for 2 days. The reaction quenched by addition of aqueous saturated NaHCO₃, transferred to a separatory funnel, and extracted 3 times with DCM. The combined organic phases were dried over sodium sulfate, filtered and concentrated. The residue was purified by flash chromatography to give 2 as an off-white solid (1.1 g, 65% yield) LCMS (ESI): m/z calculated for C₁₈H₂₁N₃O₇S: 423.4, found 424.1 (M+1); 446.1 (M+Na).

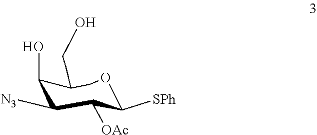

Compound 3: Compound 2 (1.1 g, 2.60 mmoles) was dissolved in methanol (25 mL) at room temperature. Sodium methoxide (0.1 mL, 25% sol. in MeOH) was added and the reaction mixture was stirred at room temperature for 2 hours. The reaction mixture neutralized by the addition of Amberlyst acidic resin, filtered and concentrated to give crude 3, which was used for the next step without further purification. LCMS (ESI): m/z calculated for C₂H¹⁵N₃O₄S: 297.3, found 298.1 (M+1); 320.1 (M+Na).

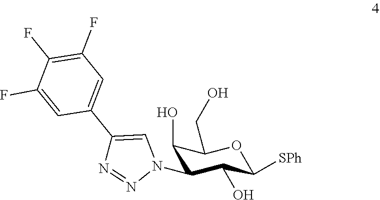

Compound 4: Crude compound 3 (2.60 mmoles), 3,4,5-trifluorophenyl-1-acetylene (2.5 equiv), THPTA (0.11 equiv), and copper (II) sulfate (0.1) were dissolved in methanol (15 mL) at room temperature. Sodium ascorbate (2.4 equiv) dissolved in water was added and the reaction mixture was stirred overnight at room temperature. The resultant precipitate was collected by filtration, washed with hexanes and water, and dried to give compound 4 as a pale yellow solid (1.2 g, 1000% yield for 2 steps). LCMS (ESI): m/z calculated for C₂₀H₁₈F₃N₃O₄S: 453.1, found 454.2 (M+1); 476.2 (M+Na).

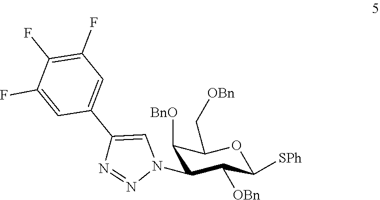

Compound 5: Compound 4 (1.2 g, 2.65 mmoles) was dissolved in DMF (15 mL) and cooled on an ice bath. Sodium hydride (60% oil dispersion, 477 mg, 11.93 mmoles) was added and the mixture stirred for 30 minutes. Benzyl bromide (1.42 mL, 11.93 mmoles) was added and the reaction was warmed to room temperature and stirred overnight. The reaction mixture was quenched by the addition of aqueous saturated ammonium chloride solution, transferred to a separatory funnel and extracted 3 times with ether. The combined organic phases were dried over magnesium sulfate, filtered, and concentrated. The residue was purified by flash chromatography to afford compound 5 (1.8 g, 94% yield). LCMS (ESI): m/z calculated for C₄₁H₃₆F₃N₃O₄S: 723.2, found 724.3 (M+1); 746.3 (M+Na).

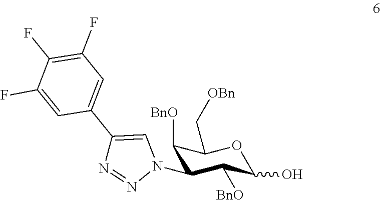

Compound 6: Compound 5 (1.8 g, 2.49 mmol) was dissolved in acetone (20 mL) and water (2 mL) and cooled on an ice bath. Trichloroisocyanuric acid (637 mg, 2.74 mmoles) was added and the reaction mixture stirred on the ice bath for 3 h. The acetone was removed in vacuo and the residue was diluted with DCM, transferred to a separatory funnel, and washed with saturated aqueous NaHCO₃. The organic phase was concentrated and the residue was purified by flash chromatography to afford compound 6 (1.5 g, 95%). LCMS (ESI): m/z calculated for C₃₅H₃₂N₃O₅: 631.2, found 632.2 (M+1); 654.2 (M+Na).

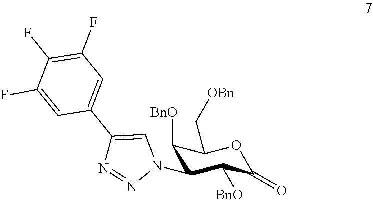

Compound 7: Compound 6 (1.0 g, 1.58 mmoles) was dissolved in DCM (20 mL) and cooled on an ice bath. Dess-Martin periodinane (1.0 g, 2.37 mmoles) was added and mixture was allowed to warm to room temperature and stirred overnight. The reaction mixture quenched by the addition of aqueous saturated NaHCO₃, transferred to a separatory funnel, and extracted 2 times with DCM. The combined organic phases were dried over sodium sulfate, filtered, and concentrated. The residue was purified by flash chromatography to afford compound 7 (520 mg, 52% yield). LCMS (ESI): m/z calculated for C₃₅H₃₀F₃N₃O₅: 629.2, found 652.2 (M+Na); 662.2 (M+MeOH+1); 684.2 (M+MeOH+Na).

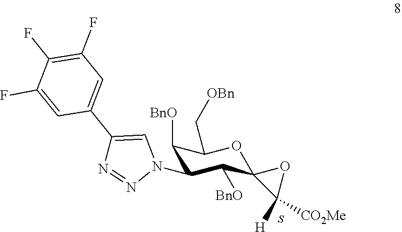

Compound 8: Methyl bromoacetate (253 mg, 1.65 mmoles) dissolved in 0.5 mL of THF was added dropwise to a solution of lithium bis(trimethylsilyl)amide (1.0 M in THF, 1.65 mL, 1.65 mmoles) cooled at −78 C. The reaction mixture was stirred for 30 minutes at −78 C. Compound 7 (260 mg, 0.41 mmoles) dissolved in THF (2.0 mL) was then added. The reaction mixture was stirred at −78 C for 30 minutes. The reaction was quenched by the addition of aqueous saturated NH₄Cl and warmed to rt. The reaction mixture was transferred to a separatory funnel and extracted 3 times with ethyl acetate. The combined organic phases were dried over sodium sulfate, filtered and concentrated. The residue was separated by flash chromatography to afford compound 8 (183 mg, 64% yield).

¹H NMR (400 MHz, Chloroform-d) δ 7.38-7.22 (m, 9H), 7.15-7.11 (m, 3H), 7.09 (dd, J=8.4, 6.6 Hz, 1H), 7.06-7.00 (m, 2H), 6.98-6.93 (m, 2H), 5.11 (dd, J=11.3, 3.2 Hz, 1H), 4.60 (d, J=11.8 Hz, 1H), 4.57-4.49 (m, 2H), 4.49-4.42 (m, 2H), 4.35 (d, J=11.8 Hz, 1H), 4.14 (d, J=3.2 Hz, 1H), 4.05 (s, 1H), 4.02 (d, J=7.0 Hz, 1H), 3.84 (d, J=11.0 Hz, 1H), 3.81 (s, 3H), 3.70 (dd, J=9.5, 7.7 Hz, 1H), 3.62 (dd, J=9.4, 6.0 Hz, 1H). LCMS (ESI): m/z calculated for C₃₈H₃₄F₃N₃O₇: 701.2, found 702.3 (M+1); 724.3 (M+Na).

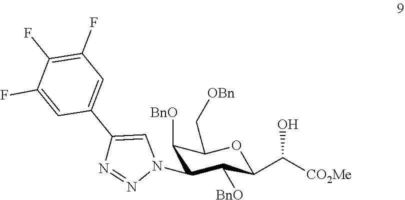

Compound 9: Compound 8 (5.0 g, 7.13 mmol) was azeotroped with toluene two times under reduced pressure, and then dried under high vacuum for 2 hours. It was then dissolved in anhydrous CH₂Cl₂ (125 mL) and cooled on an ice bath while stirring under an atmosphere of argon. Tributyltin hydride (15.1 mL, 56.1 mmol) was added dropwise and the solution was allowed to stir for 25 minutes on the ice bath. Trimethylsilyl triflate (2.1 mL, 11.6 mmol) dissolved in 20 mL of anhydrous CH₂Cl₂ was then added dropwise over the course of 5 minutes. The reaction was slowly warmed to ambient temperature and stirred for 16 hours. The reaction mixture was then diluted with CH₂Cl₂ (50 mL), transferred to a separatory funnel, and washed with saturated aqueous NaHCO₃ (50 mL). The aqueous phase was separated and extracted with CH₂Cl₂ (50 mL×2). The combined organic phases were washed with saturated aqueous NaHCO₃ (50 mL), dried over Na₂SO₄, filtered, and concentrated. The residue was purified by flash chromatography (hexanes to 40% EtOAc in hexanes, gradient) to afford compound 9 (2.65 g, 48%).

¹H-NMR (400 MHz, CDCl₃): δ 7.65 (s, 1H), 7.36-7.22 (m, 8H), 7.16-7.06 (m, 7H), 6.96-6.90 (m, 2H), 5.03 (dd, J=10.7, 3.2 Hz, 1H), 4.72 (d, J=2.3 Hz, 1H), 4.51 (dt, J=22.6, 11.4 Hz, 3H), 4.41 (d, J=10.9 Hz, 1H), 4.32 (dd, J=10.7, 9.2 Hz, 1H), 4.07 (d, J=3.1 Hz, 1H), 3.94 (d, J=10.9 Hz, 1H), 3.92-3.84 (m, 3H), 3.78-3.71 (m, 4H), 3.65 (dd, J=9.1, 5.5 Hz, 1H), 0.24 (s, 9H). LCMS (ESI): m/z calculated for C₄₁H₄₄F₃N₃O₇Si: 775.8, found 798.2 (M+Na).

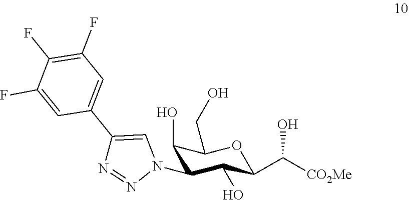

Compound 10: To a solution of compound 9 (2.65 g, 3.4 mmol) was in anhydrous MeOH (40 mL) was added Pd(OH)₂ (0.27 g, 20% by wt). The mixture was cooled on an ice bath and stirred for 30 minutes. Triethylsilane (22 mL, 137 mmol) was added dropwise. The solution was allowed to slowly warm to ambient temperature and stirred for 16 hours. The reaction mixture was filtered through a bed of Celite and concentrated. The residue was purified by flash chromatography (hexanes to 100% EtOAc, gradient) to afford compound 10 (1.09 g, 73%).

¹H-NMR (400 MHz, CD₃OD): δ 8.57 (s, 1H), 7.77-7.53 (m, 2H), 4.91-4.82 (m, 1H), 4.66-4.59 (m, 1H), 4.55 (dd, J=10.8, 9.4 Hz, 1H), 4.13 (d, J=2.8 Hz, 1H), 3.86 (dd, J=9.4, 2.1 Hz, 1H), 3.81 (s, 3H), 3.77-3.74 (m, 1H), 3.71-3.68 (m, 2H). LCMS (ESI): m/z calculated for C₁₇H₁₈F₃N₃O: 433.3, found 456.0 (M+Na).

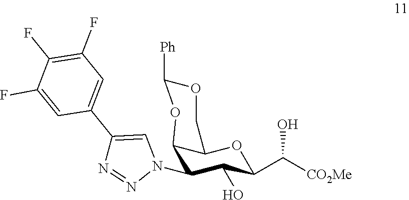

Compound 11: Compound 10 (1.09 g, 2.5 mmol) and CSA (0.115 g, 0.49 mmol) were suspended in anhydrous MeCN (80 mL) under an argon atmosphere. Benzaldehyde dimethyl acetal (0.45 mL, 2.99 mmol) was added dropwise. The reaction mixture was allowed to stir for 16 hours at ambient temperature, during which time it became a homogenous solution. The reaction mixture was then neutralized with a few drops of Et₃N, and concentrated. The residue was purified via flash chromatography (CH₂Cl₂ to 10% MeOH in CH₂Cl₂, gradient) to afford compound 11 (978 mg, 75/).

¹H NMR (400 MHz, DMSO-d6): δ 8.84 (s, 1H), 7.95-7.73 (m, 2H), 7.33 (qdt, J=8.4, 5.6, 2.7 Hz, 5H), 5.51 (t, J=3.8 Hz, 2H), 5.47 (d, J=6.8 Hz, 1H), 5.14 (dd, J=10.8, 3.6 Hz, 1H), 4.54 (dd, J=6.7, 2.2 Hz, 1H), 4.47 (ddd, J=10.8, 9.3, 7.5 Hz, 1H), 4.40 (d, J=4.0 Hz, 1H), 4.09-3.99 (m, 2H), 3.85 (dd, J=9.3, 2.2 Hz, 1H), 3.81-3.76 (m, 1H), 3.71 (s, 3H). LCMS (ESI): m/z calculated for C₂₄H₂₂F₃N₃O₇: 521.4, found 544.1 (M+Na).

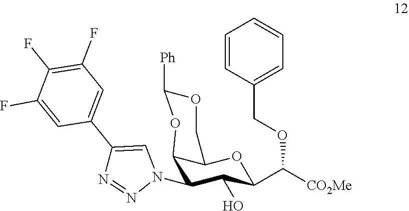

Compound 12: Compound 11 (25.2 mg, 0.048 mmol) was azeotroped with toluene 2 times under reduced pressure, dried under high vacuum for 2 hours, then dissolved in anhydrous DMF (2 mL) and cooled on an ice bath. Benzyl bromide (6 uL, 0.05 mmol) dissolved in 0.5 mL of anhydrous DMF was added and the reaction and was stirred under an atmosphere of argon for 30 minutes at 0° C. Sodium hydride (2 mg, 0.05 mmol, 60%) was added and the reaction was allowed to gradually warm to ambient temperature while stirring for 16 hours. The reaction mixture was diluted with EtOAc (20 mL), transferred to a separatory funnel, and washed with H₂O (10 mL). The aqueous phase was separated and extracted with EtOAc (10 mL×3). The combined organic phases were washed with H₂O (10 mL×3), dried over Na₂SO₄, filtered, and concentrated. The residue was purified via preparative TLC (5% MeOH in CH₂Cl₂) to afford compound 12 (6.3 mg, 21%). LCMS (ESI): m/z calculated for C₃₁H₂₈F₃N₃O₇: 611.5, found 634.1 (M+Na).

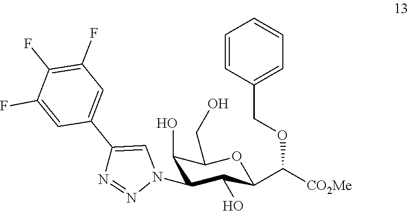

Compound 13: Compound 12 (6.3 mg, 0.01 mmol) was dissolved in anhydrous MeOH (1 mL) containing CSA (0.26 mg, 0.001 mmol). The reaction mixture was heated to 76° C. in a screw-cap scintillation vial while stirring. After 2 hours, an additional 0.13 mg of CSA in 0.5 mL of MeOH was added. The reaction mixture was stirred at 76° C. for 16 hours. The reaction mixture concentrated under reduced pressure. The residue was purified via preparative TLC (10% MeOH in CH₂Cl₂) to afford compound 13 (4.2 mg, 80%).

¹H NMR (400 MHz, DMSO-d₆) δ 8.80 (s, 1H), 7.94-7.86 (m, 2H), 7.48-7.42 (m, 2H), 7.38 (t, J=7.4 Hz, 2H), 7.36-7.28 (m, 1H), 5.46 (d, J=7.7 Hz, 1H), 5.28 (d, J=6.0 Hz, 1H), 4.85 (dd, J=10.7, 2.9 Hz, 1H), 4.67 (d, J=11.0 Hz, 1H), 4.62-4.58 (m, 1H), 4.54 (d, J=11.1 Hz, 1H), 4.44 (d, J=2.5 Hz, 1H), 4.36 (q, J=9.5 Hz, 1H), 3.95-3.90 (m, 1H), 3.78 (dd, J=9.3, 2.5 Hz, 1H), 3.71 (s, 3H), 3.61-3.54 (m, 1H), 3.52-3.43 (m, 1H), 3.43-3.38 (m, 1H). LCMS (ESI): m/z calculated for C₂₄H₂₄F₃N₃O₇: 523.4, found 546.0 (M+Na).

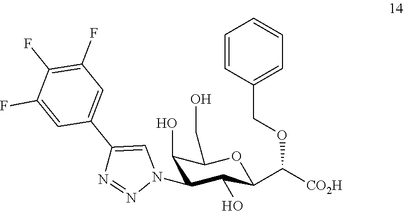

Compound 14: To a solution of compound 13 (3.5 mg, 0.007 mmoles) in methanol (0.5 mL) was added 1.0 M NaOH solution (0.1 mL). The reaction mixture was stirred overnight at room temperature then neutralized with acidic resin, filtered and concentrated. The residue was purified by reverse phase chromatography using a C—8 matrix to afford 3.0 mg compound 14 (90%). LCMS (ESI): m/z calculated for C₂₃H₂₂F₃N₃O₇: 509.1, found 508.2 (M-H).

### Example 2

### Synthesis of Building Blocks 15-35 and 76

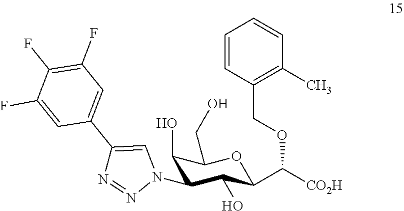

Prophetic Compound 15: Compound 15 is prepared according to FIG. 1 by substituting 2-methyl benzyl bromide for benzyl bromide in step k.

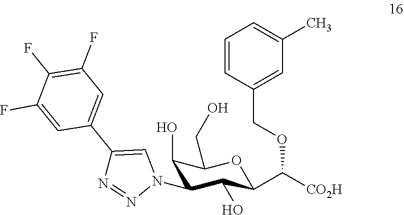

Prophetic Compound 16: Compound 16 is prepared according to FIG. 1 by substituting 3-methyl benzyl bromide for benzyl bromide in step k.

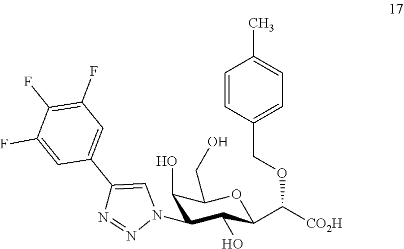

Prophetic Compound 17: Compound 17 is prepared according to FIG. 1 by substituting 4-methyl benzyl bromide for benzyl bromide in step k.

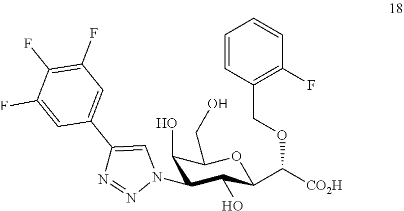

Prophetic Compound 18: Compound 18 is prepared according to FIG. 1 by substituting 2-fluoro benzyl bromide for benzyl bromide in step k.

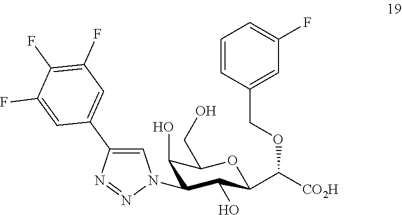

Prophetic Compound 19: Compound 19 is prepared according to FIG. 1 by substituting 3-fluoro benzyl bromide for benzyl bromide in step k.

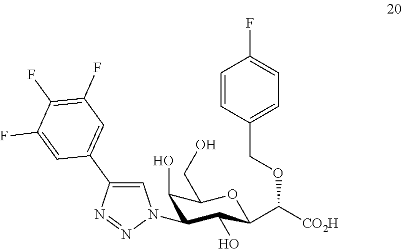

Prophetic Compound 20: Compound 20 is prepared according to FIG. 1 by substituting 4-fluoro benzyl bromide for benzyl bromide in step k.

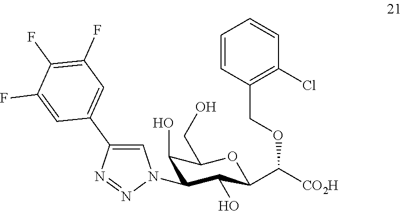

Prophetic Compound 21: Compound 21 is prepared according to FIG. 1 by substituting 2-chloro benzyl bromide for benzyl bromide in step k.

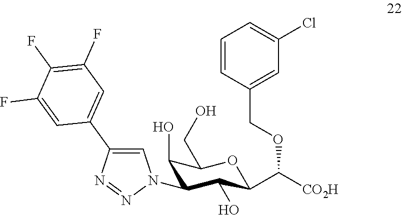

Prophetic Compound 22: Compound 22 is prepared according to FIG. 1 by substituting 3-chloro benzyl bromide for benzyl bromide in step k.

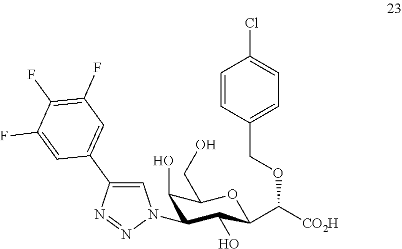

Prophetic Compound 23: Compound 23 is prepared according to FIG. 1 by substituting 4-chloro benzyl bromide for benzyl bromide in step k.

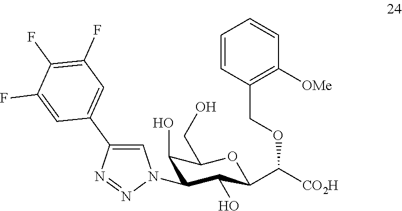

Prophetic Compound 24: Compound 24 is prepared according to FIG. 1 by substituting 2-methoxy benzyl bromide for benzyl bromide in step k.

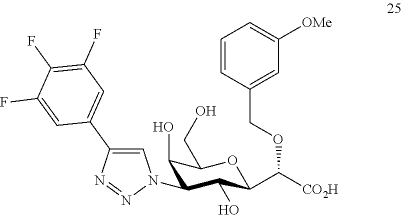

Prophetic Compound 25: Compound 25 is prepared according to FIG. 1 by substituting 3-methoxy benzyl bromide for benzyl bromide in step k.

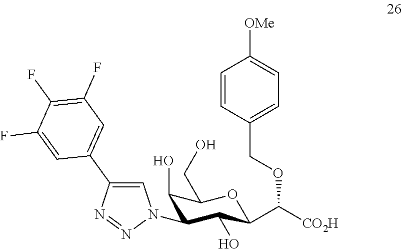

Prophetic Compound 26: Compound 26 is prepared according to FIG. 1 by substituting 4-methoxy benzyl chloride for benzyl bromide in step k.

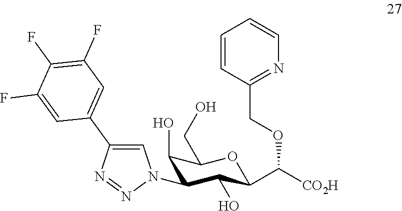

Prophetic Compound 27: Compound 27 is prepared according to FIG. 1 by substituting 2-picolyl bromide for benzyl bromide in step k.

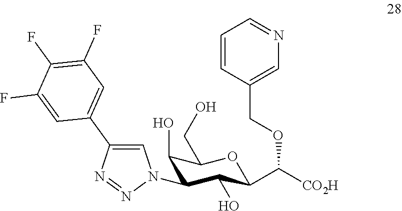

Prophetic Compound 28: Compound 28 is prepared according to FIG. 1 by substituting 3-picolyl bromide for benzyl bromide in step k.

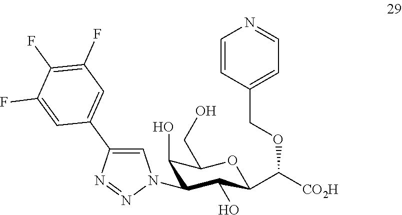

Prophetic Compound 29: Compound 29 is prepared according to FIG. 1 by substituting 4-picolyl bromide for benzyl bromide in step k.

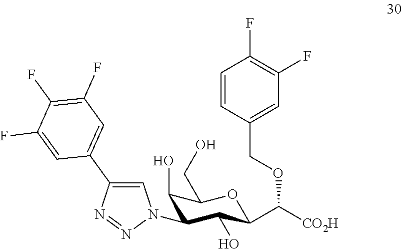

Prophetic Compound 30: Compound 30 is prepared according to FIG. 1 by substituting 3,4-difluoro benzyl bromide for benzyl bromide in step k.

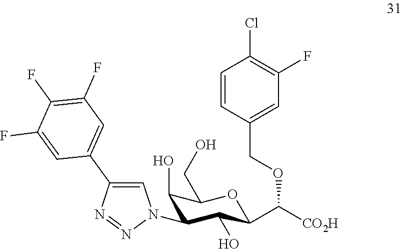

Prophetic Compound 31: Compound 31 is prepared according to FIG. 1 by substituting 3-fluoro, 4-chloro benzyl bromide for benzyl bromide in step k.

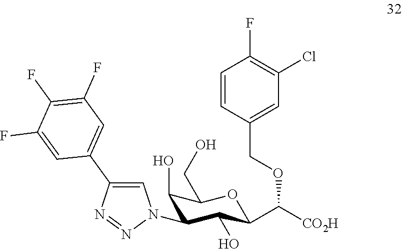

Prophetic Compound 32: Compound 32 is prepared according to FIG. 1 by substituting 3-chloro, 4-fluoro benzyl bromide for benzyl bromide in step k.

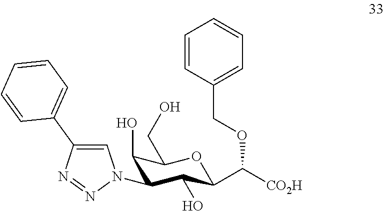

Prophetic Compound 33: Compound 33 is prepared according to FIG. 1 by substituting phenyl acetylene for 3, 4, 5-trifluorophenyl-1-acetylene in step d.

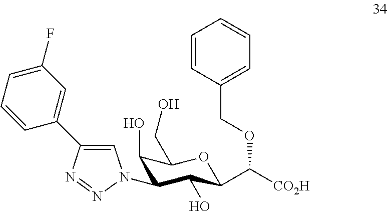

Prophetic Compound 34: Compound 34 is prepared according to FIG. 1 by substituting 3-fluorophenyl acetylene for 3, 4, 5-trifluorophenyl-1-acetylene in step d.

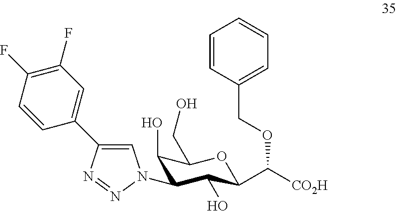

Prophetic Compound 35: Compound 35 is prepared according to FIG. 1 by substituting 3,4-difluorophenyl-1-acetylene for 3, 4, 5-trifluorophenyl-1-acetylene in step d.

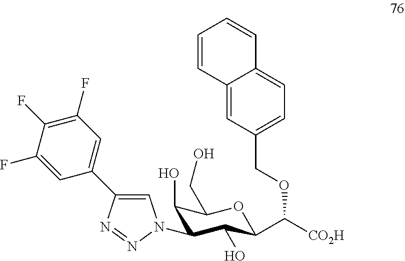

Prophetic Compound 76: Compound 76 is prepared according to FIG. 1 by substituting 2-naphthyl bromide for benzyl bromide in step k.

### Example 3

### Synthesis of Building Block 37

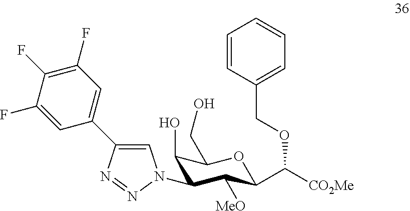

Prophetic Compound 36: Compound 12 is dissolved in DMF and cooled on an ice bath. Sodium hydride (1.1 eq) is added and the reaction mixture is stirred for 30 minutes. Methyl iodide (1.1 eq) is added and the reaction mixture is stirred until completion. The reaction mixture is quenched by the addition of saturated ammonium chloride, transferred to a separatory funnel, and extracted 3 times with ethyl acetate. The combined organic phases are dried over sodium sulfate, filtered and concentrated. The residue is separated by flash chromatography to afford compound 36.

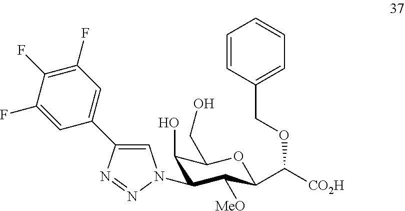

Prophetic Compound 37: Compound 36 is dissolved in methanol. Sodium hydroxide (3 eq of a 1M solution) is added and the reaction mixture is stirred at room temperature until completion. The pH is adjusted to 2 with 1 M HCl and the solvent removed. The residue is purified by flash chromatography to afford compound 37.

### Example 4

### Synthesis of Building Block 44

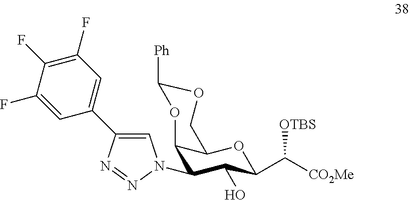

Prophetic Compound 38: Compound 11 is dissolved in DMF and cooled on an ice bath. Imidazole (1.1 eq) is added followed by TBSCl (1.1 eq). The reaction mixture is stirred until completion. The reaction mixture is diluted with ethyl acetate, transferred to a separatory funnel and washed 3 times with water. The organic phase is dried over sodium sulfate, filtered, and concentrated. The residue is separated by flash chromatography to afford compound 38.

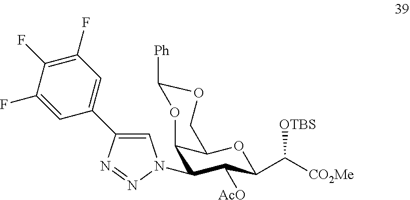

Prophetic Compound 39: Compound 38 is dissolved in pyridine and cooled on an ice bath. Acetic anhydride (2 eq) is added and the reaction mixture is stirred until completion. The reaction mixture is diluted with ethyl acetate, transferred to a separatory funnel and washed 3 times with water. The organic phase is dried over sodium sulfate, filtered, and concentrated. The residue is separated by flash chromatography to afford compound 39.

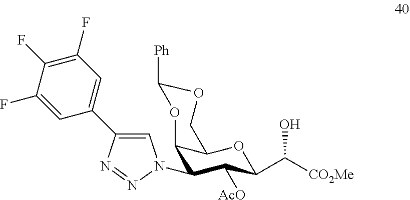

Prophetic Compound 40: Compound 39 is dissolved in THF and cooled on an icebath. Tetrabutylammonium fluoride (1.1 eq) is added and the reaction mixture is stirred until completion. The reaction mixture is diluted with ethyl acetate, transferred to a separatory funnel and washed 3 times with water. The organic phase is dried over magnesium sulfate, filtered, and concentrated. The residue is separated by flash chromatography to afford compound 40.

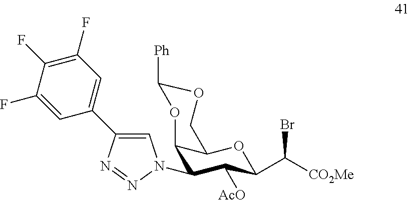

Prophetic Compound 41: Compound 40 is dissolved in DCM and cooled on an ice bath. Thionyl bromide (1.1 eq) is added and the reaction mixture is stirred until completion. The reaction mixture is quenched by the addition of saturated aqueous sodium bicarbonate solution. The reaction mixture is transferred to a separatory funnel and extracted 3 times with ethyl acetate. The combined organic phases are dried over sodium sulfate, filtered and concentrated. The residue is separated by flash chromatography to afford compound 41.

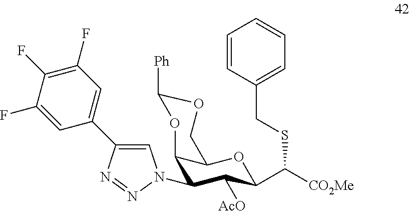

Prophetic Compound 42: Benzyl thiol is dissolved in DMF and cooled to room temperature. Sodium hydride (1.05 eq) is added and the reaction mixture is stirred for 30 minutes. Compound 41 (0.9 eq) dissolved in DMF is added. The reaction mixture is stirred until completion. The reaction mixture is quenched by the addition of saturated aqueous ammonium chloride solution. The reaction mixture is transferred to a separatory funnel and extracted 3 times with ethyl acetate. The combined organic phases are dried over sodium sulfate, filtered and concentrated. The residue is separated by flash chromatography to afford compound 42.

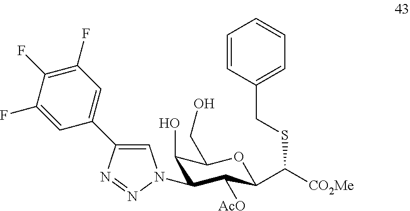

Prophetic Compound 43: Compound 42 is dissolved in methanol. A catalytic amount of CSA is added and the reaction mixture is refluxed overnight. The reaction mixture is concentrated and the residue is purified by flash chromatography to afford compound 43.

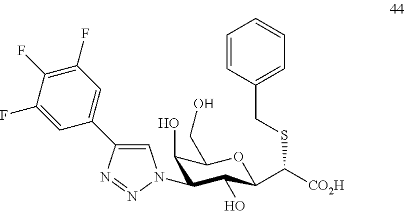

Prophetic Compound 44: Compound 43 is dissolved in methanol. Sodium hydroxide (3 eq of a 1M solution) is added and the reaction mixture is stirred at room temperature until completion. The pH is adjusted to 2 with 1 M HCl and the solvent removed. The residue is purified by flash chromatography to afford compound 44.

### Example 5

### Synthesis of Building Block 49

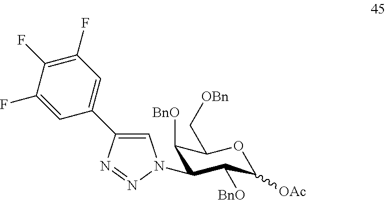

Prophetic Compound 45: Compound 6 is dissolved in pyridine and cooled on an ice bath. Acetic anhydride (2 eq) is added and the reaction mixture is stirred until completion. The reaction mixture is diluted with ethyl acetate, transferred to a separatory funnel and washed 3 times with water. The organic phase is dried over sodium sulfate, filtered, and concentrated. The residue is separated by flash chromatography to afford compound 45.

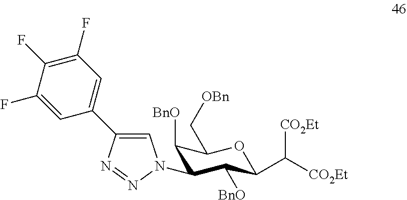

Prophetic Compound 46: Compound 45 is dissolved in DCM and cooled on an ice bath. Trimethylsilyl iodide (1.1 eq) is added and the reaction mixture is stirred until completion. The solvent is removed in vacuo and the residue is dissolved in THF. In a separate flask diethyl malonate is dissolved in THF and NaHMDS (0.9 eq) and 15-crown-5 (0.9 eq) are added. After stirring for 30 minutes the glycosyl iodide solution is added and the reaction mixture is stirred until completion. The solvent is removed and the residue is purified by flash chromatography to afford compound 46.

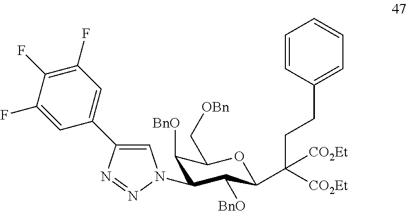

Prophetic Compound 47: Compound 46 is dissolved in DMF (15 mL) and cooled on an ice bath. Sodium hydride (1.1 eq) is added and the mixture stirred for 30 minutes. Phenethyl bromide (1.1 eq) is added and the reaction was warmed to room temperature and stirred until completion. The reaction mixture is quenched by the addition of aqueous saturated ammonium chloride solution, transferred to a separatory funnel and extracted 3 times with ether. The combined organic phases are dried over magnesium sulfate, filtered, and concentrated. The residue is purified by flash chromatography to afford compound 47.

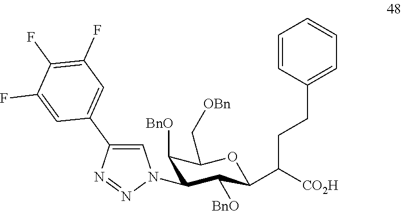

Prophetic Compound 48: Compound 47 is dissolved in THF. Sodium hydroxide (5 eq of a 1M aqueous solution) is added and the reaction mixture is stirred at 50° C. until completion. A large excess of acetic acid is added and the reaction mixture is stirred at 100° C. until completion. The solvent is removed and the residue is purified by flash chromatography to afford compound 48.

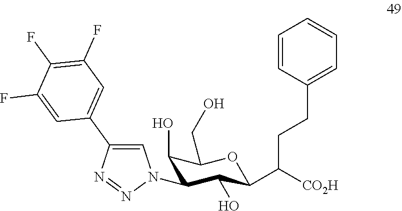

Prophetic Compound 49: To a solution of compound 48 dissolved in methanol is added Pd/C. The reaction mixture is hydrogenated on a Paar shaker until completion. The reaction mixture is filtered through Celite and concentrated to afford compound 49.

### Example 6

### Synthesis of Compound 51

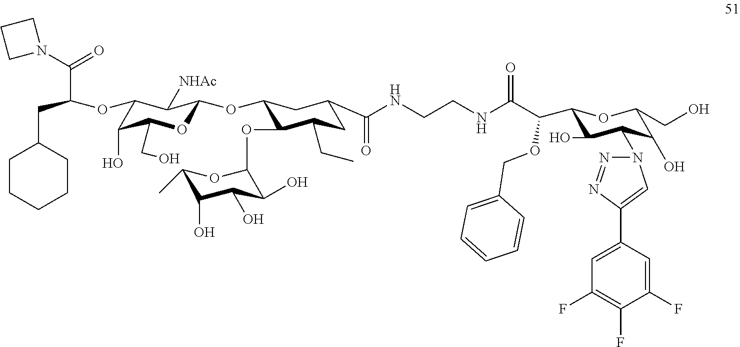

Prophetic Compound 51: To a solution of compound 14 in anhydrous DMF can be added HATU (1 eq) and DIPEA (1.2 eq). The mixture can be stirred at ambient temperature for 15 minutes followed by addition of compound 50 (1.2 eq) (preparation described in WO2018068010). The reaction mixture can be stirred at ambient temperature until completion. The reaction mixture can be concentrated in vacuo and the residue purified by HPLC to afford compound 51.

### Example 7

### Synthesis of Compounds 52-72

Prophetic Compounds 52-72: Compounds 52-72 are prepared from compound 50 using the procedures outlined for compound 51 and in FIGS. 1, 2, and 3.

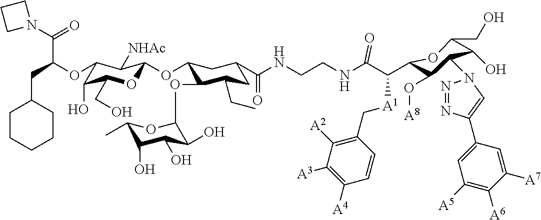

| Cmpd | A¹ | A² | A³ | A⁴ | A⁵ | A⁶ | A⁷ | A⁸ |
|---|---|---|---|---|---|---|---|---|
| 52 | O | CH₃ | H | H | F | F | F | H |
| 53 | O | H | CH₃ | H | F | F | F | H |
| 54 | O | H | H | CH₃ | F | F | F | H |
| 55 | O | F | H | H | F | F | F | H |
| 56 | O | H | F | H | F | F | F | H |
| 57 | O | H | H | F | F | F | F | H |
| 58 | O | Cl | H | H | F | F | F | H |
| 59 | O | H | Cl | H | F | F | F | H |
| 60 | O | H | H | Cl | F | F | F | H |
| 61 | O | OCH₃ | H | H | F | F | F | H |
| 62 | O | H | OCH₃ | H | F | F | F | H |
| 63 | O | H | H | OCH₃ | F | F | F | H |
| 64 | O | H | F | F | F | F | F | H |
| 65 | O | H | F | Cl | F | F | F | H |
| 66 | O | H | Cl | F | F | F | F | H |
| 67 | O | H | H | H | H | H | H | H |
| 68 | O | H | H | H | F | H | H | H |
| 69 | O | H | H | H | F | F | H | H |
| 70 | O | H | H | H | F | F | F | CH₃ |
| 71 | S | H | H | H | F | F | F | H |
| 72 | CH₂ | H | H | H | F | F | F | H |

### Example 8

### Synthesis of Compound 73

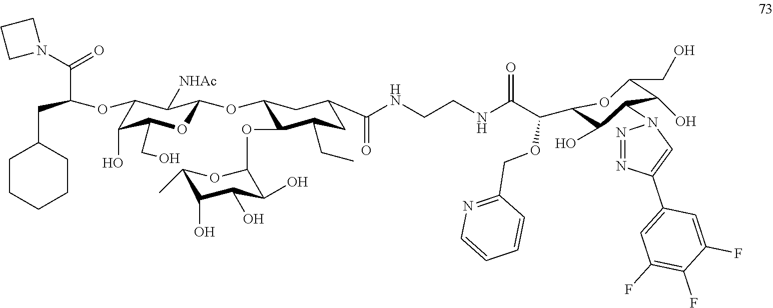

Prophetic Compound 73: Compound 73 is prepared from compound 50 using the procedures outlined for compound 51 and in FIGS. 1, 2, and 3.

### Example 9

### Synthesis of Compound 74

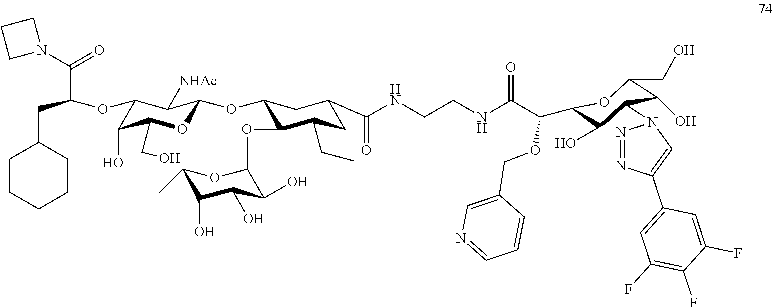

Prophetic Compound 74: Compound 74 is prepared from compound 50 using the procedures outlined for compound 51 and in FIGS. 1, 2, and 3.

### Example 10

### Synthesis of Compound 75

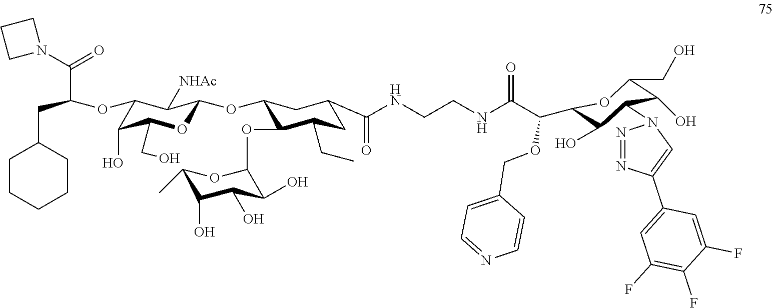

Prophetic Compound 75: Compound 75 is prepared from compound 50 using the procedures outlined for compound 51 and in FIGS. 1, 2, and 3.

### Example 11

### Synthesis of Compound 77

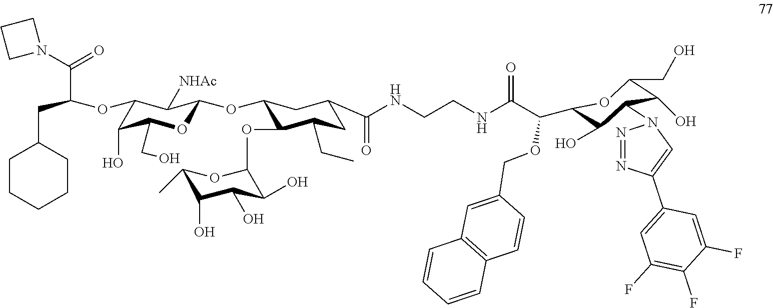

Prophetic Compound 77: Compound 77 is prepared from compound 50 using the procedures outlined for compound 51 and in FIGS. 1, 2, and 3.

### Example 12

### Synthesis of Compound 87

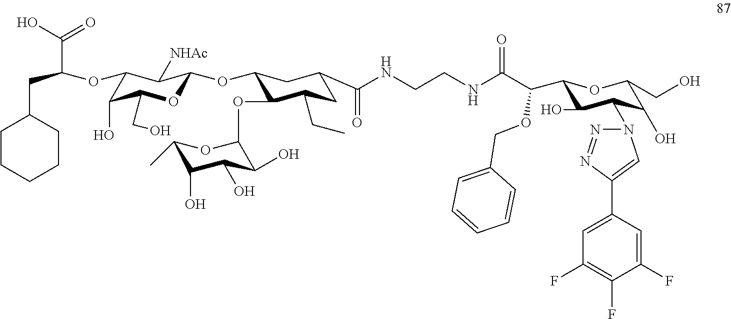

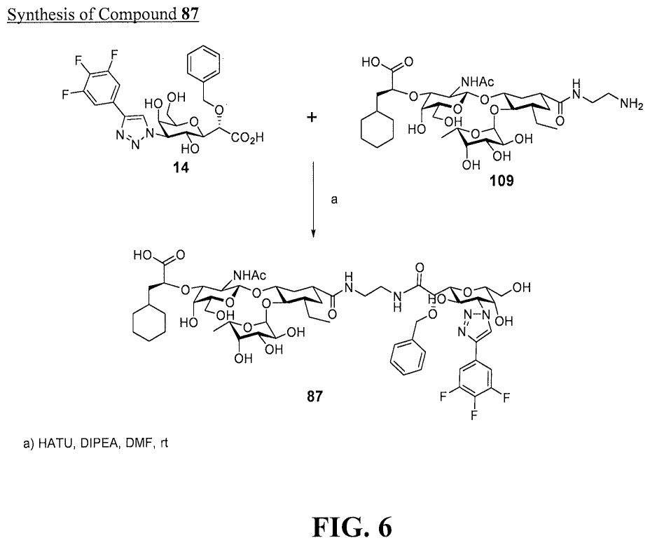

Prophetic Compound 87: To a solution of compound 14 in anhydrous DMF is added HATU (1 eq) and DIPEA (1.2 eq). The mixture is stirred at ambient temperature for 15 minutes followed by addition of compound 109 (1.2 eq) (see FIG. 6; preparation described in WO2013096926). The reaction mixture is stirred at ambient temperature until completion. The reaction mixture is concentrated in vacuo and the residue purified by HPLC to afford compound 87.

### Example 13

### Synthesis of Compound 88-108

Prophetic Compounds 88-108: Compounds 88-108 are prepared from compound 109 using the procedures outlined for Compound 87 and in FIGS. 1, 2, and 3.

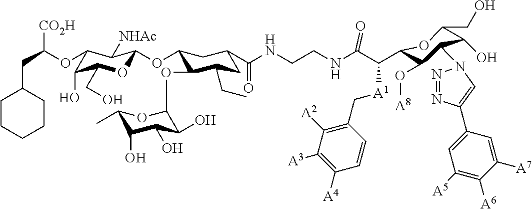

| Cmpd | A¹ | A² | A³ | A⁴ | A⁵ | A⁶ | A⁷ | A⁸ |
|---|---|---|---|---|---|---|---|---|
| 88 | O | CH₃ | H | H | F | F | F | H |
| 89 | O | H | CH₃ | H | F | F | F | H |
| 90 | O | H | H | CH₃ | F | F | F | H |
| 91 | O | F | H | H | F | F | F | H |
| 92 | O | H | F | H | F | F | F | H |
| 93 | O | H | H | F | F | F | F | H |
| 94 | O | Cl | H | H | F | F | F | H |
| 95 | O | H | Cl | H | F | F | F | H |
| 96 | O | H | H | Cl | F | F | F | H |
| 97 | O | OCH₃ | H | H | F | F | F | H |
| 98 | O | H | OCH₃ | H | F | F | F | H |
| 99 | O | H | H | OCH₃ | F | F | F | H |
| 100 | O | H | F | F | F | F | F | H |
| 101 | O | H | F | Cl | F | F | F | H |
| 102 | O | H | Cl | F | F | F | F | H |
| 103 | O | H | H | H | H | H | H | H |
| 104 | O | H | H | H | F | H | H | H |
| 105 | O | H | H | H | F | F | H | H |
| 106 | O | H | H | H | F | F | F | CH₃ |
| 107 | S | H | H | H | F | F | F | H |
| 108 | CH₂ | H | H | H | F | F | F | H |

### Example 14

### Synthesis of Compound 83

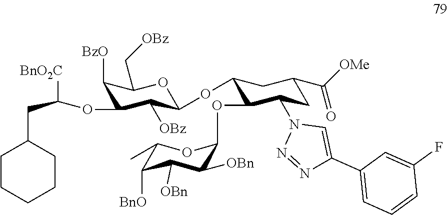

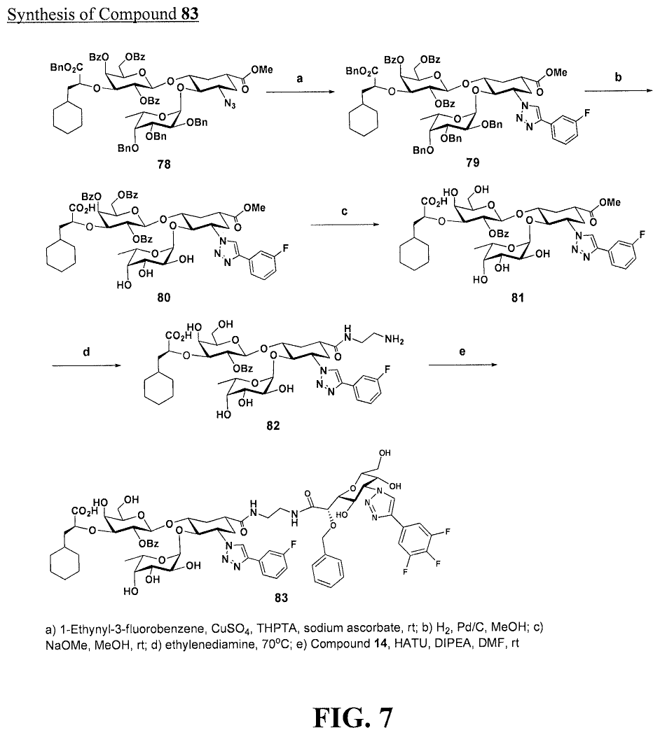

Prophetic Compound 79: To a solution of compound 78 (see FIG. 7; preparation described in WO 2007/028050)) and 1-ethynyl-3-fluorobenzene (1.2 equivalents) in methanol is added a stock solution of copper sulfate and tris(3-hydroxypropyltriazolylmethyl) amine in water (0.2 equivalents). The reaction is initiated by addition of an aqueous solution of sodium ascorbate (1.3 equivalents). After stirring at ambient temperature for an appropriate length of time the solvent is removed in vacuo. The product is purified by normal phase column chromatography to give the compound 79.

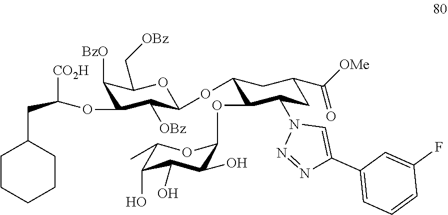

Prophetic Compound 80: A mixture of compound 79 and a catalytic amount of Pd/C in MeOH is stirred at room temperature under a H₂ atmosphere (balloon). After reaction is complete, the mixture is filtered through Celite® and concentrated to afford compound 80.

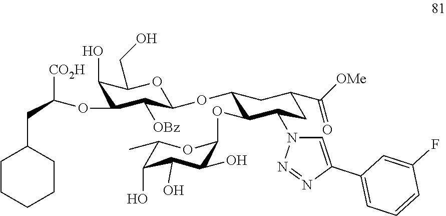

Prophetic Compound 81: To a solution of compound 80 in MeOH at room temperature is added a catalytic amount of NaOMe in MeOH. The reaction mixture is stirred at room temperature until completion. The pH is adjusted to ˜4 by addition of acetic acid. The reaction mixture is concentrated and the residue separated by flash chromatography to afford compound 81.

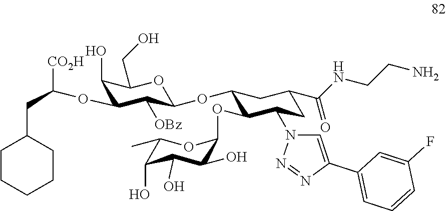

Compound 82: Compound 81 is dissolved in ethylenediamine (10 equivalents) under an atmosphere of argon and stirred at 70° C. until the reaction is complete. The reaction mixture is cooled to room temperature then co-evaporated with methanol and toluene. The residue is purified by HPLC to give compound 82.

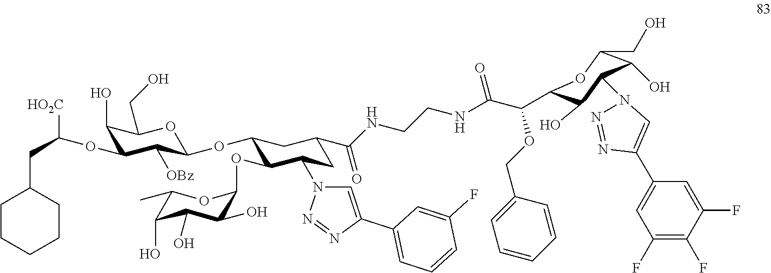

Prophetic Compound 83: To a solution of compound 14 in anhydrous DMF is added HATU (1.1 equivalents) and DIPEA (1.3 equivalents). The mixture is stirred at ambient temperature for 15 minutes followed by addition of compound 82 (see FIG. 7; 1 equivalent). The mixture is stirred at ambient temperature until completion. The solvent is removed in vacuo and the residue is purified by HPLC to afford compound 83.

### Example 15

### Synthesis of Compound 83

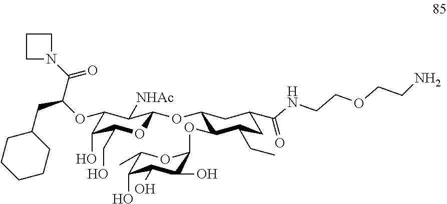

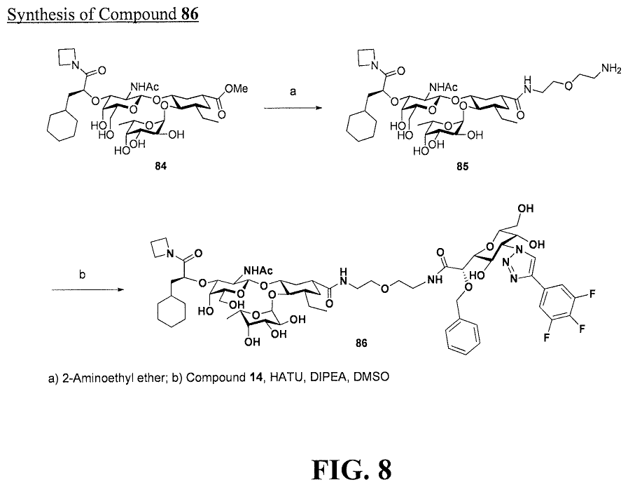

Prophetic Compound 85: Compound 84 (see FIG. 8; preparation described in WO2018068010) is heated at 70° C. overnight with 2-aminoethyl amine (10 eq). After cooling to room temperature the reaction mixture is separated by reverse phase chromatography to afford compound 85.

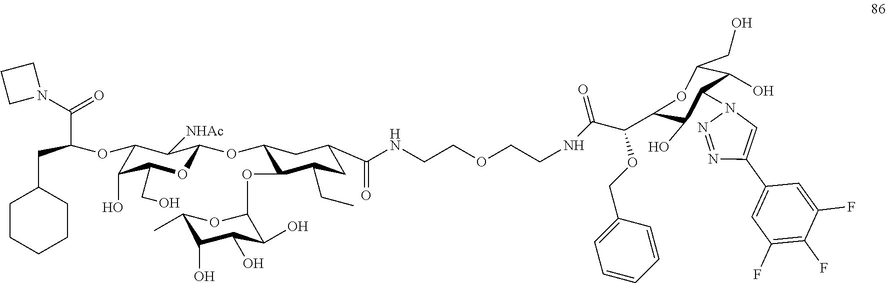

Prophetic Compound 86: Compound 86 is synthesized from compound 85 and compound 14 following the procedure as described for the synthesis of compound 83.

### Example 16

### E-Selectin Activity—Binding Assay

The inhibition assay to screen for and characterize antagonists of E-selectin is a competitive binding assay, which allows the determination of IC₅₀ values. E-selectin/Ig chimera is immobilized in 96 well microtiter plates by incubation at 37° C. for 2 hours. To reduce nonspecific binding, bovine serum albumin is added to each well and incubated at room temperature for 2 hours. The plate is washed and serial dilutions of the test compounds are added to the wells in the presence of conjugates of biotinylated, sLe^{a} polyacrylamide with streptavidin/horseradish peroxidase and incubated for 2 hours at room temperature.

To determine the amount of sLe^{a} bound to immobilized E-selectin after washing, the peroxidase substrate, 3,3′,5,5′ tetramethylbenzidine (TMB) is added. After 3 minutes, the enzyme reaction is stopped by the addition of H₃PO₄, and the absorbance of light at a wavelength of 450 nm is determined. The concentration of test compound required to inhibit binding by 50% is determined.

### Example 17

### Galectin-3 Activity—ELISA Assay

Galectin-3 antagonists is evaluated for their ability to inhibit binding of galectin-3 to a Galβ1-3GlcNAc carbohydrate structure. The detailed protocol is as follows. A 1 ug/mL suspension of a Galβ1-3GlcNAcβ1-3Galβ1-4GlcNAcβ-PAA-biotin polymer (Glycotech, catalog number 01-096) is prepared. A 100 uL aliquot of the polymer is added to the wells of a 96-well streptavidin-coated plate (R&D Systems, catalog number CP004). A 100 uL aliquot of 1× Tris Buffered Saline (TBS, Sigma, catalog number T5912-10X) is added to control wells. The polymer is allowed to bind to the streptavidin-coated wells for 1.5 hours at room temperature. The contents of the wells is discarded and 200 uL of 1×TBS containing 1% bovine serum albumin (BSA) is added to each well as a blocking reagent and the plate is kept at room temperature for 30 minutes. The wells are washed three times with 1×TBS containing 0.1% BSA. A serial dilution of test compounds is prepared in a separate V-bottom plate (Corning, catalog number 3897). A 75 uL aliquot of the highest concentration of the compound to be tested is added to the first well in a column of the V-bottom plate then 15 ul are serially transferred into 60 uL 1×TBS through the remaining wells in the column to generate a 1 to 5 serial dilution. A 60 uL aliquot of 2 ug/mL galectin-3 (IBL, catalog number IBATGP0414) is added to each well in the V-bottom plate. A 100 uL aliquot of the galectin-3/test compound mixture is transferred from the V-bottom plate into the assay plate containing the Galβ1-3GlcNAc polymer. Four sets of control wells in the assay plate are prepared in duplicate containing 1) both Galβ1-3GlcNAc polymer and galectin-3, 2) neither the polymer nor galectin-3, 3) galectin-3 only, no polymer, or 4) polymer only, no galectin-3. The plate is gently rocked for 1.5 hours at room temperature. The wells are washed four times with TBS/0.1% BSA. A 100 uL aliquot of anti-galectin-3 antibody conjugated to horse radish peroxidase (R&D Systems, from DGAL30 kit) is added to each well and the plate is kept at room temperature for 1 hour. The wells are washed four times with TBS/0.1% BSA. A 100 uL aliquot of TMB substrate solution is added to each well. The TMB substrate solution is prepared by making a 1:1 mixture of TMB Peroxidase Substrate (KPL, catalog number 5120-0048) and Peroxidase Substrate Solution B (KPL, catalog number 5120-0037). The plate is kept at room temperature for 10 to 20 minutes. The color development is stopped by adding 100 uL 10% phosphoric acid (RICCA Chemical Co., catalog number 5850-16). The absorbance at 450 nm (A₄₅₀) is measured using a FlexStation 3 plate reader (Molecular Devices). Plots of A₄₅₀ versus test compound concentration and IC₅₀ determinations are made using GraphPad Prism 6.

1-62. (canceled)

63. At least one compound chosen from compounds of Formula (I): 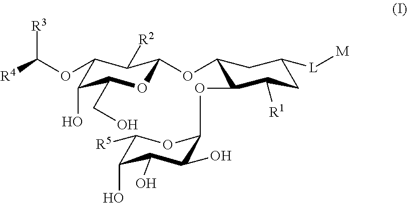
and pharmaceutically acceptable salts thereof, wherein
R¹ is chosen from methyl, ethyl, 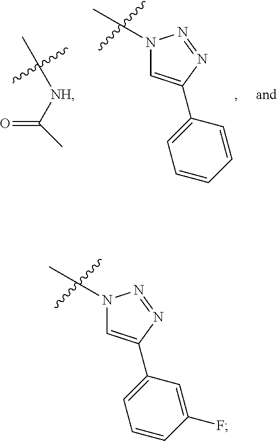
R² is chosen from 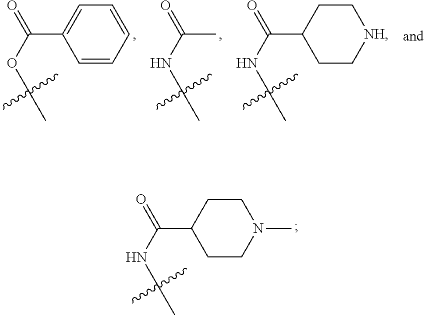
R³ is chosen from 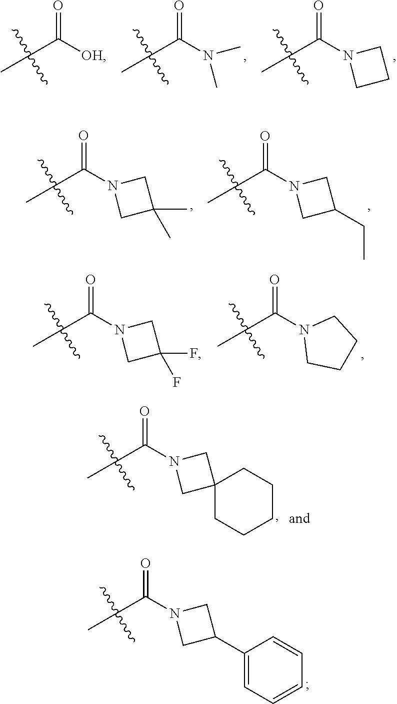
R⁴ is chosen from propyl and cyclohexylmethyl;
R⁵ is chosen from CN, CF₃, and methyl;
M is chosen from 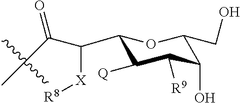
groups,
wherein
X is chosen from —O—, —S—, and —CH₂;
Q is chosen from H, halo, and —OZ³ groups, wherein Z³ is chosen from H and C₁₋₈ alkyl groups, wherein Z³ is optionally substituted with one or more groups independently chosen from halo and —OR¹² groups, wherein R¹² is independently chosen from H and C₁₋₈ alkyl groups;
R⁸ is chosen from 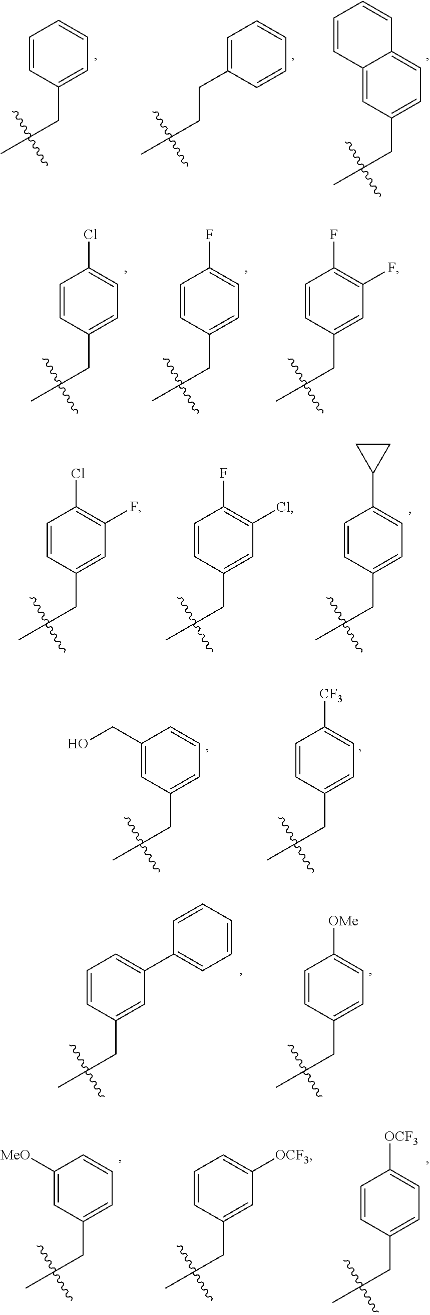 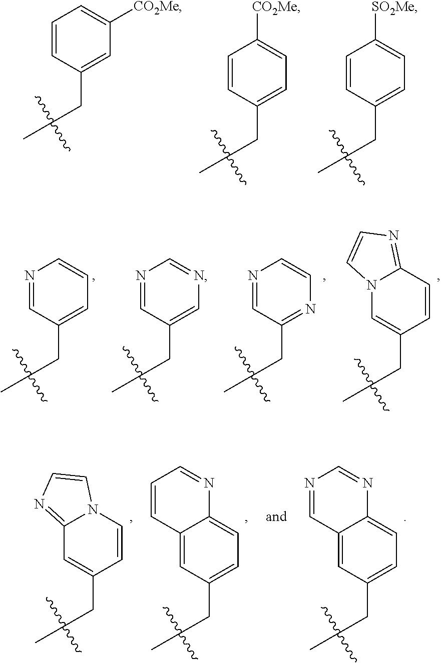
R⁹ is chosen from 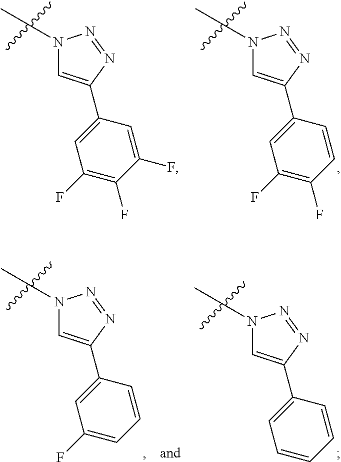 [SMILES: *n1cc(-c2cc(F)c(F)c(F)c2)nn1 ; *n1cc(-c2ccc(F)c(F)c2)nn1 ; *n1cc(-c2cccc(F)c2)nn1 ; *n1cc(-c2ccccc2)nn1]
and L is a linker group chosen from

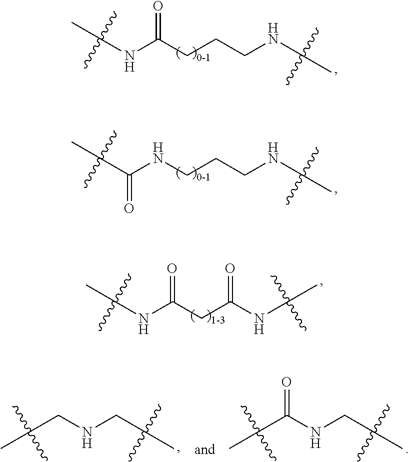

64. The at least one compound according to claim 63, wherein R² is chosen from

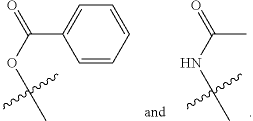

65. The at least one compound according to claim 63, wherein R³ is chosen from

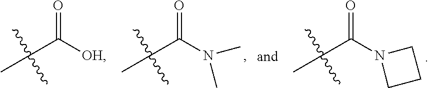

66. The at least one compound according to claim 63, wherein R⁵ is methyl.

67. The at least one compound according to claim 63, wherein M is chosen from 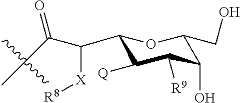
groups.

68. The at least one compound according to claim 63, wherein M is chosen from 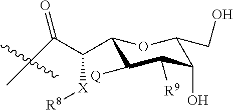
groups.

69. The at least one compound according to claim 63, wherein X is —CH₂—.

70. The at least one compound according to claim 63, wherein X is —O—.

71. The at least one compound according to claim 63, wherein X is —S—.

72. The at least one compound according to claim 63, wherein R⁹ is chosen from

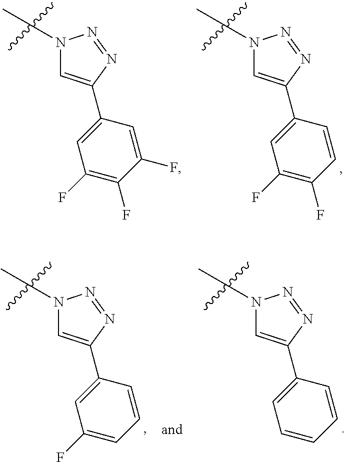 [SMILES: *n1cc(-c2cc(F)c(F)c(F)c2)nn1 ; *n1cc(-c2ccc(F)c(F)c2)nn1 ; *n1cc(-c2cccc(F)c2)nn1 ; *n1cc(-c2ccccc2)nn1]

73. The at least one compound according to claim 63, wherein the linker group is

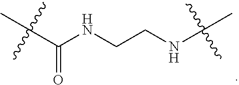 [SMILES: *NCCNC(*)=O]

74. The at least one compound according to claim 63, chosen from compounds having the following Formulae: 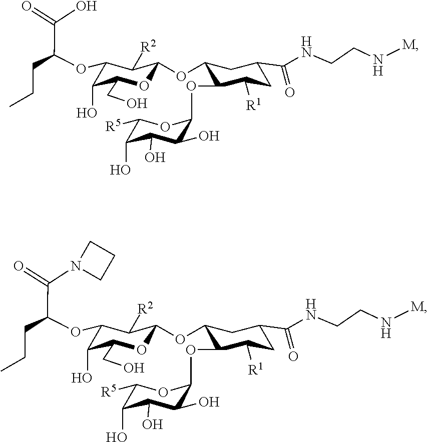
and pharmaceutically acceptable salts of any of the foregoing.

75. The at least one compound according to claim 63, chosen from compounds having the following Formulae: 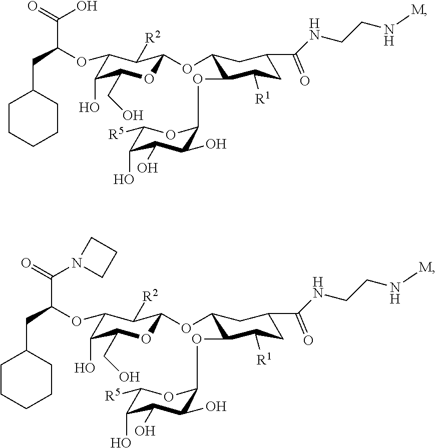
and pharmaceutically acceptable salts of any of the foregoing.

76. The at least one compound according to claim 63, chosen from compounds having the following Formulae: 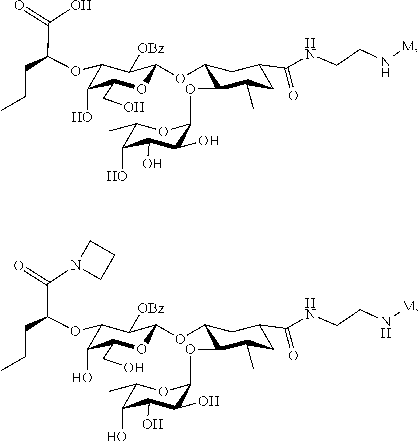
and pharmaceutically acceptable salts of any of the foregoing.

77. The at least one compound according to claim 63, chosen from compounds having the following Formulae: 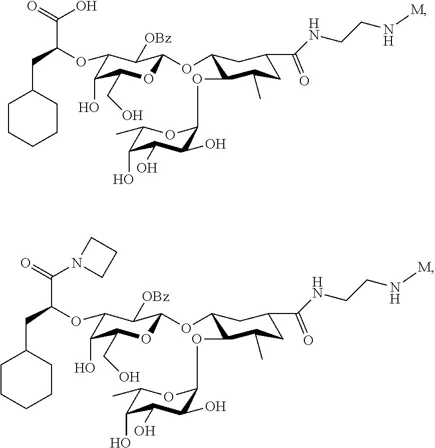
and pharmaceutically acceptable salts of any of the foregoing.

78. The at least one compound according to claim 63, chosen from compounds having the following Formulae: 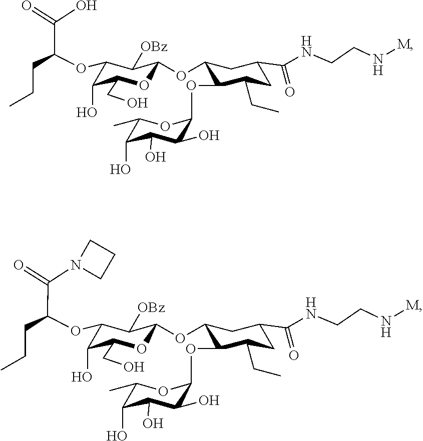
and pharmaceutically acceptable salts of any of the foregoing.

79. The at least one compound according to claim 63, chosen from compounds having the following Formulae: 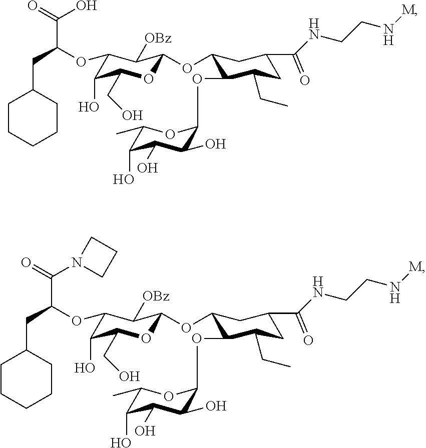
and pharmaceutically acceptable salts of any of the foregoing.

80. The at least one compound according to claim 63, chosen from compounds having the following Formulae: 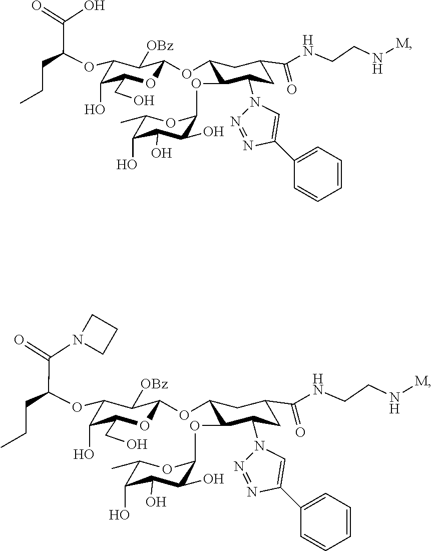
and pharmaceutically acceptable salts of any of the foregoing.

81. The at least one compound according to claim 63, chosen from compounds having the following Formulae: 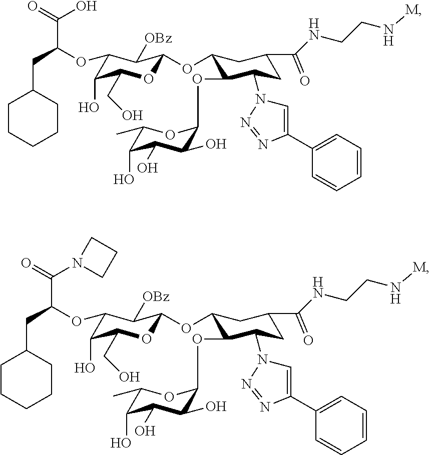
and pharmaceutically acceptable salts of any of the foregoing.

82. The at least one compound according to claim 63, chosen from compounds having the following Formulae: 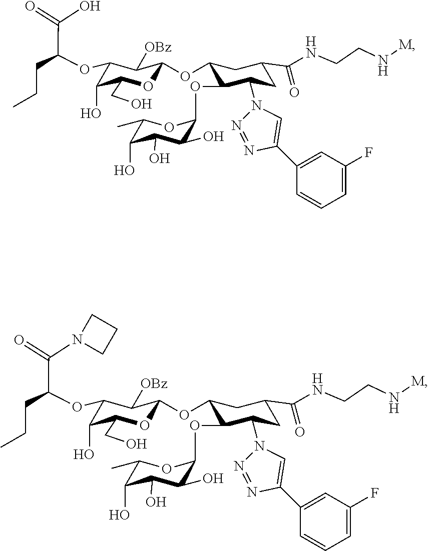
and pharmaceutically acceptable salts of any of the foregoing.

83. The at least one compound according to claim 63, chosen from compounds having the following Formulae: 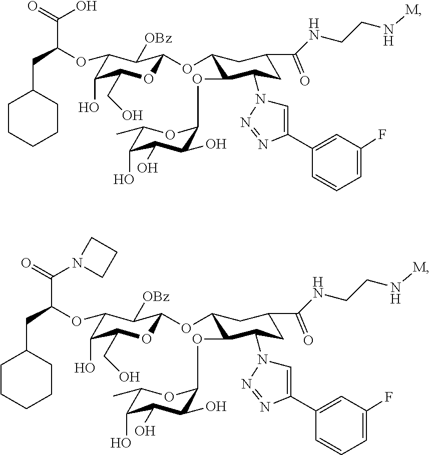
and pharmaceutically acceptable salts of any of the foregoing.

84. The at least one compound according to claim 63, chosen from compounds having the following Formulae: 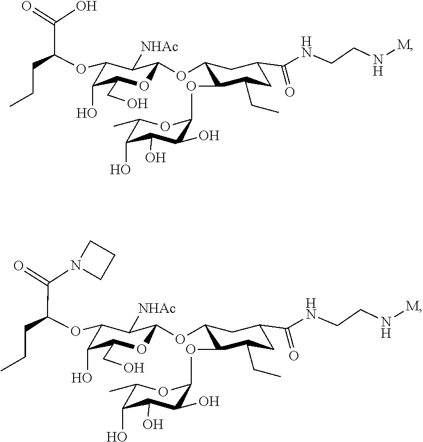
and pharmaceutically acceptable salts of any of the foregoing.

85. The at least one compound according to claim 63, chosen from compounds having the following Formulae: 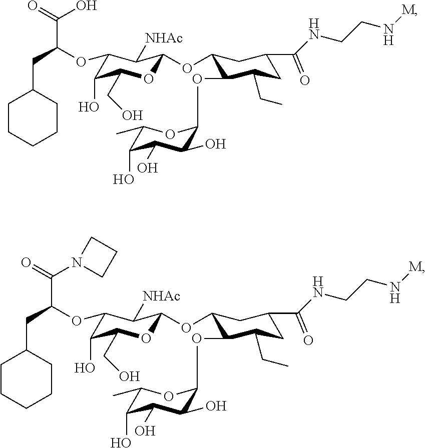
and pharmaceutically acceptable salts of any of the foregoing.

86. The at least one compound according to claim 63, chosen from compounds having the following Formulae: 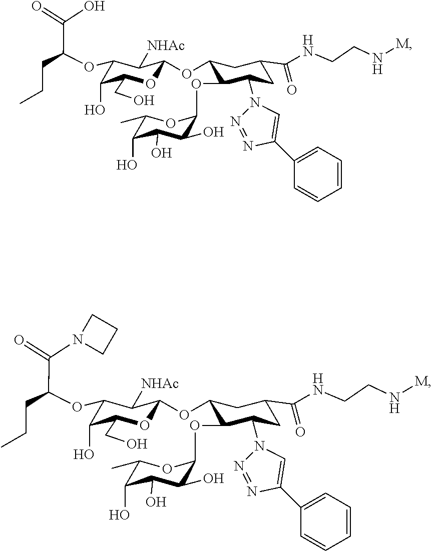
and pharmaceutically acceptable salts of any of the foregoing.

87. The at least one compound according to claim 63, chosen from compounds having the following Formulae: 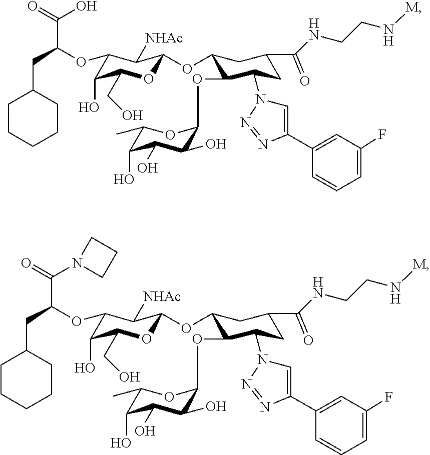
and pharmaceutically acceptable salts of any of the foregoing.

88. The at least one compound according to claim 63, chosen from compounds having the following Formulae: 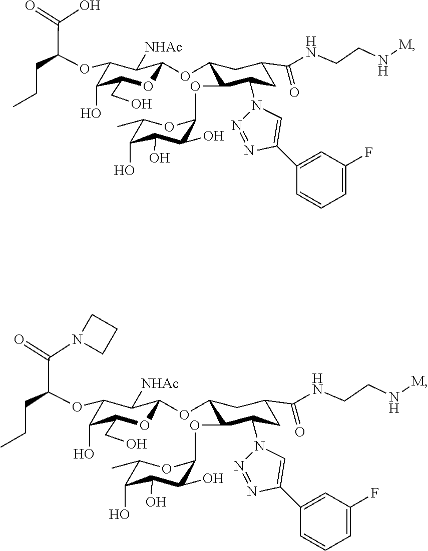
and pharmaceutically acceptable salts of any of the foregoing.

89. The at least one compound according to claim 63, chosen from compounds having the following Formulae: 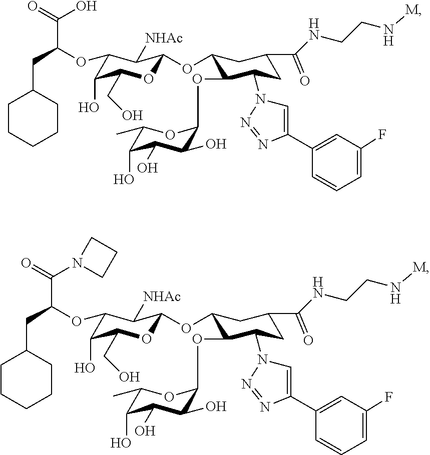
and pharmaceutically acceptable salts of any of the foregoing.

90. The at least one compound according to claim 63, chosen from compounds having the following Formulae: 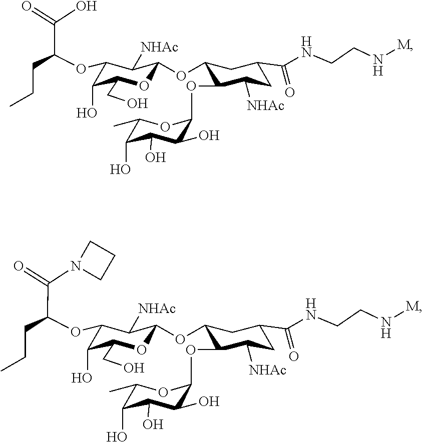
and pharmaceutically acceptable salts of any of the foregoing.

91. The at least one compound according to claim 63, chosen from compounds having the following Formulae: 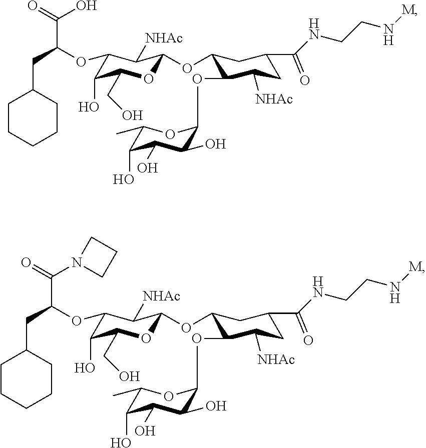
and pharmaceutically acceptable salts of any of the foregoing.

92. A composition comprising the at least one compound of claim 63 and at least one additional pharmaceutically acceptable ingredient.

93. A method for treatment of at least one inflammatory disease, disorder, and/or condition, the method comprising administering to a subject in need thereof an effective amount of at least one compound of claim 63.

94. A method for treatment of cancer, the method comprising administering to a subject in need thereof an effective amount of at least one compound of claim 63.

95. The method according to claim 94, wherein the cancer is chosen from solid tumor cancers.

96. The method according to claim 94, wherein the cancer is chosen from bone cancers, colorectal cancers, and pancreatic cancers.

97. The method according to claim 94, wherein the cancer is chosen from liquid tumor cancers.

98. The method according to claim 94, wherein the cancer is chosen from acute myelogenous leukemia, acute lymphoblastic leukemia, chronic myelogenous leukemia, and multiple myeloma.

99. A method for treatment of cancer, the method comprising administering to a subject in need thereof (a) an effective amount of at least one compound of claim 63 and (b) at least one therapy chosen from (i) chemotherapy and (ii) radiotherapy.

100. A method for treatment of metastasis of cancer cells, the method comprising administering to a subject in need thereof an effective amount of at least one compound of claim 63.

101. A method for inhibiting infiltration of cancer cells into the liver, lymph nodes, lung, bone, and/or bone marrow, the method comprising administering to a subject in need thereof an effective amount of at least one compound of claim 63.

102. A method for enhancing hematopoietic stem cell survival, the method comprising administering to a subject in need thereof an effective amount of at least one compound of claim 63.

103. The method according to claim 102, wherein the subject has cancer and has received or will receive chemotherapy and/or radiotherapy.

104. A method for mobilizing cells from the bone marrow, the method comprising administering to a subject in need thereof an effective amount of at least one compound of claim 63.

105. The method according to claim 104, wherein the cells are chosen from hematopoietic cells and tumor cells.

106. A method for treatment of mucositis, the method comprising administering to a subject in need thereof an effective amount of at least one compound of claim 63.

107. The method according to claim 106, wherein the mucositis is chosen from oral mucositis, esophageal mucositis, and gastrointestinal mucositis.

108. The method according to claim 106, wherein the subject is afflicted with head and neck, breast, lung, ovarian, prostate, lymphatic, leukemic, and/or gastrointestinal cancer.

109. A method for treatment of thrombosis, the method comprising administering to a subject in need thereof an effective amount of at least one compound of claim 63.

110. A method for treatment of at least one cardiovascular disease or complications associated therewith, the method comprising administering to a subject in need thereof an effective amount of at least one compound of claim 63.

111. The method according to claim 110, wherein the at least one cardiovascular disease is chosen from atherosclerosis and myocardial infarction.

112. A method of inhibiting rejection of a transplanted tissue in a subject, wherein said subject is a recipient of the transplanted tissue, the method comprising administering to a subject in need thereof an effective amount of at least one compound of claim 63.

113. A method for treatment of graft versus host disease or complications associated therewith, the method comprising administering to a subject in need thereof an effective amount of at least one compound of claim 63.

114. A method for treatment of pathological angiogenesis, the method comprising administering to a subject in need thereof an effective amount of at least one compound of claim 63.

115. The method according to claim 114, wherein the pathological angiogenesis occurs in the eye.

116. The method according to claim 114, wherein the pathological angiogenesis occurs in a subject with cancer.

117. A method for treatment of an epileptic syndrome, the method comprising administering to a subject in need thereof an effective amount of at least one compound of claim 63.

118. A method for treatment of neurodegeneration, the method comprising administering to a subject in need thereof an effective amount of at least one compound of claim 63.

119. The method according to claim 118, wherein the neurodegenerative disease is an α-synucleinopathy.

120. A method for treatment of fibrosis, the method comprising administering to a subject in need thereof an effective amount of at least one compound of claim 63.

121. The method according to claim 120, wherein the fibrosis is pulmonary fibrosis.

122. The method according to claim 120, wherein the fibrosis is cardiac fibrosis.

123. A method for treatment of liver disorders or complications associated therewith, the method comprising administering to a subject in need thereof an effective amount of at least one compound of claim 63.

124. The method according to claim 123, wherein the liver disorder is nonalcoholic steatohepatitis.
